SEQUENCE LISTING (60 sequences)

SEQ ID NO: 1 - DNA, 1311 bp, Methylomonas 16a
gatgtggtcacatggccctatcacttaacggctgatattcgattttgtcattggttttttcttaactttaacttctacacgctcatgaacaaacctaaaaaagttgcaatactgacagcaggcggcttggcgccttgtttgaattccgcaatcggtagtttgatcgaacgttataccgaaatcgatcctagcatagaaatcatttgctatcgcggcggttataaaggcctgttgctgggcgattcttatccagtaacggccgaagtgcgtaaaaaggcgggtgttctgcaacgttttggcggttctgtgatcggcaacagccgcgtcaaattgaccaatgtcaaagactgcgtgaaacgcggtttggtcaaagagggtgaagatccgcaaaaagtcgcggctgatcaattggttaaggatggtgtcgatattctgcacaccatcggcggcgatgataccaatacggcagcagcggatttggcagcattcctggccagaaataattacggactgaccgtcattggtttacctaaaaccgtcgataacgacgtatttccgatcaagcaatcactaggtgcttggactgccgccgagcaaggcgcgcgttatttcatgaacgtggtggccgaaaacaacgccaacccacgcatgctgatcgtacacgaagtgatgggccgtaactgcggctggctgaccgctgcaaccgcgcaggaatatcgcaaattactggaccgtgccgagtggttgccggaattgggtttgactcgtgaatcttatgaagtgcacgcggtattcgttccggaaatggcgatcgacctggaagccgaagccaagcgcctgcgcgaagtgatggacaaagtcgattgcgtcaacatcttcgtttccgaaggtgccggcgtcgaagctatcgtcgcggaaatgcaggccaaaggccaggaagtgccgcgcgatgcgttcggccacatcaaactggatgcggtcaaccctggtaaatggttcggcgagcaattcgcgcagatgataggcgcggaaaaaaccctggtacaaaaatcgggatacttcgcccgtgcttctgcttccaacgttgacgacatgcgtttgatcaaatcgtgcgccgacttggcggtcgagtgcgcgttccgccgcgagtctggcgtgatcggtcacgacgaagacaacggcaacgtgttgcgtgcgatcgagtttccgcgcatcaagggcggcaaaccgttcaatatcgacaccgactggttcaatagcatgttgagcgaaatcggccagcctaaaggcggtaaagtcgaagtcagccac

SEQ ID NO: 2 - protein, 437 aa, Methylomonas 16a
DVVTWPYHLTADIRFCHWFFLNFNFYTLMNKPKKVAILTAGGLAPCLNSAIGSLIERYTEIDPSIEIICYRGGYKGLLLGDSYPVTAEVRKKAGVLQRFGGSVIGNSRVKLTNVKDCVKRGLVKEGEDPQKVAADQLVKDGVDILHTIGGDDTNTAAADLAAFLARNNYGLTVIGLPKTVDNDVFPIKQSLGAWTAAEQGARYFMNVVAENNANPRMLIVHEVMGRNCGWLTAATAQEYRKLLDRAEWLPELGLTRESYEVHAVFVPEMAIDLEAEAKRLREVMDKVDCVNIFVSEGAGVEAIVAEMQAKGQEVPRDAFGHIKLDAVNPGKWFGEQFAQMIGAEKTLVQKSGYFARASASNVDDMRLIKSCADLAVECAFRRESGVIGHDEDNGNVLRAIEFPRIKGGKPFNIDTDWFNSMLSEIGQPKGGKVEVSH

SEQ ID NO: 3 - DNA, 636 bp, Methylomonas 16a
gaaaatactatgtccgtcaccatcaaagaagtcatgaccacctcgcccgttatgccggtcatggtcatcaatcatctggaacatgccgtccctctggctcgcgcgctagtcgacggtggcttgaaagttttggagatcacattgcgcacgccggtggcactggaatgtatccgacgtatcaaagccgaagtaccggacgccatcgtcggcgcgggcaccatcatcaaccctcataccttgtatcaagcgattgacgccggtgcggaattcatcgtcagccccggcatcaccgaaaatctactcaacgaagcgctagcatccggcgtgcctatcctgcccggcgtcatcacacccagcgaggtcatgcgtttattggaaaaaggcatcaatgcgatgaaattctttccggctgaagccgccggcggcataccgatgctgaaatcccttggcggccccttgccgcaagtcaccttctgtccgaccggcggcgtcaatcccaaaaacgcgcccgaatatctggcattgaaaaatgtcgcctgcgtcggcggctcctggatggcgccggccgatctggtagatgccgaagactgggcggaaatcacgcggcgggcgagcgaggccgcggcattgaaaaaa

SEQ ID NO: 4 - protein, 212 aa, Methylomonas 16a
ENTMSVTIKEVMTTSPVMPVMVINHLEHAVPLARALVDGGLKVLEITLRTPVALECIRRIKAEVPDAIVGAGTIINPHTLYQAIDAGAEFIVSPGITENLLNEALASGVPILPGVITPSEVMRLLEKGINAMKFFPAEAAGGIPMLKSLGGPLPQVTFCPTGGVNPKNAPEYLALKNVACVGGSWMAPADLVDAEDWAEITRRASEAAALKK

SEQ ID NO: 5 - DNA, 1860 bp, Methylomonas 16a
atgaaactgaccaccgactatcccttgcttaaaaacatccacacgccggcggacatacgcgcgctgtccaaggaccagctccagcaactggctgacgaggtgcgcggctatctgacccacacggtcagcatttccggcggccattttgcggccggcctcggcaccgtggaactgaccgtggccttgcattatgtgttcaatacccccgtcgatcagttggtctgggacgtgggccatcaggcctatccgcacaagattctgaccggtcgcaaggagcgcatgccgaccattcgcaccctgggcggggtgtcagcctttccggcgcgggacgagagcgaatacgatgccttcggcgtcggccattccagcacctcgatcagcgcggcactgggcatggccattgcgtcgcagctgcgcggcgaagacaagaagatggtagccatcatcggcgacggttccatcaccggcggcatggcctatgaggcgatgaatcatgccggcgatgtgaatgccaacctgctggtgatcttgaacgacaacgatatgtcgatctcgccgccggtcggggcgatgaacaattatctgaccaaggtgttgtcgagcaagttttattcgtcggtgcgggaagagagcaagaaagctctggccaagatgccgtcggtgtgggaactggcgcgcaagaccgaggaacacgtgaagggcatgatcgtgcccggtaccttgttcgaggaattgggcttcaattatttcggcccgatcgacggccatgatgtcgagatgctggtgtcgaccctggaaaatctgaaggatttgaccgggccggtattcctgcatgtggtgaccaagaagggcaaaggctatgcgccagccgagaaagacccgttggcctaccatggcgtgccggctttcgatccgaccaaggatttcctgcccaaggcggcgccgtcgccgcatccgacctataccgaggtgttcggccgctggctgtgcgacatggcggctcaagacgagcgcttgctgggcatcacgccggcgatgcgcgaaggctctggtttggtggaattctcacagaaatttccgaatcgctatttcgatgtcgccatcgccgagcagcatgcggtgaccttggccgccggccaggcctgccagggcgccaagccggtggtggcgatttattccaccttcctgcaacgcggttacgatcagttgatccacgacgtggccttgcagaacttagatatgctctttgcactggatcgtgccggcttggtcggcccggatggaccgacccatgctggcgcctttgattacagctacatgcgctgtattccgaacatgctgatcatggctccagccgacgagaacgagtgcaggcagatgctgaccaccggcttccaacaccatggcccggcttcggtgcgctatccgcgcggcaaagggcccggggcggcaatcgatccgaccctgaccgcgctggagatcggcaaggccgaagtcagacaccacggcagccgcatcgccattctggcctggggcagcatggtcacgcctgccgtcgaagccggcaagcagctgggcgcgacggtggtgaacatgcgtttcgtcaagccgttcgatcaagccttggtgctggaattggccaggacgcacgatgtgttcgtcaccgtcgaggaaaacgtcatcgccggcggcgctggcagtgcgatcaacaccttcctgcaggcgcagaaggtgctgatgccggtctgcaacatcggcctgcccgaccgcttcgtcgagcaaggtagtcgcgaggaattgctcagcctggtcggcctcgacagcaagggcatcctcgccaccatcgaacagttttgcgct

SEQ ID NO: 6 - protein, 620 aa, Methylomonas 16a
MKLTTDYPLLKNIHTPADIRALSKDQLQQLADEVRGYLTHTVSISGGHFAAGLGTVELTVALHYVFNTPVDQLVWDVGHQAYPHKILTGRKERMPTIRTLGGVSAFPARDESEYDAFGVGHSSTSISAALGMAIASQLRGEDKKMVAIIGDGSITGGMAYEAMNHAGDVNANLLVILNDNDMSISPPVGAMNNYLTKVLSSKFYSSVREESKKALAKMPSVWELARKTEEHVKGMIVPGTLFEELGFNYFGPIDGHDVEMLVSTLENLKDLTGPVFLHVVTKKGKGYAPAEKDPLAYHGVPAFDPTKDFLPKAAPSPHPTYTEVFGRWLCDMAAQDERLLGITPAMREGSGLVEFSQKFPNRYFDVAIAEQHAVTLAAGQACQGAKPVVAIYSTFLQRGYDQLIHDVALQNLDMLFALDRAGLVGPDGPTHAGAFDYSYMRCIPNMLIMAPADENECRQMLTTGFQHHGPASVRYPRGKGPGAAIDPTLTALEIGKAEVRHHGSRIAILAWGSMVTPAVEAGKQLGATVVNMRFVKPFDQALVLELARTHDVFVTVEENVIAGGAGSAINTFLQAQKVLMPVCNIGLPDRFVEQGSREELLSLVGLDSKGILATIEQFCA

SEQ ID NO: 7 - DNA, 1182 bp, Methylomonas 16a
atgaaaggtatttgcatattgggcgctaccggttcgatcggtgtcagcacgctggatgtcgttgccaggcatccggataaatatcaagtcgttgcgctgaccgccaacggcaatatcgacgcattgtatgaacaatgcctggcccaccatccggagtatgcggtggtggtcatggaaagcaaggtagcagagttcaaacagcgcattgccgcttcgccggtagcggatatcaaggtcttgtcgggtagcgaggccttgcaacaggtggccacgctggaaaacgtcgatacggtgatggcggctatcgtcggcgcggccggattgttgccgaccttggccgcggccaaggccggcaaaaccgtgctgttggccaacaaggaagccttggtgatgtcgggacaaatcttcatgcaggccgtcagcgattccggcgctgtgttgctgccgatagacagcgagcacaacgccatctttcagtgcatgccggcgggttatacgccaggccatacagccaaacaggcgcgccgcattttattgaccgcttccggtggcccatttcgacggacgccgatagaaacgttgtccagcgtcacgccggatcaggccgttgcccatcctaaatgggacatggggcgcaagatttcggtcgattccgccaccatgatgaacaaaggtctcgaactgatcgaagcctgcttgttgttcaacatggagcccgaccagattgaagtcgtcattcatccgcagagcatcattcattcgatggtggactatgtcgatggttcggttttggcgcagatgggtaatcccgacatgcgcacgccgatagcgcacgcgatggcctggccggaacgctttgactctggtgtggcgccgctggatattttcgaagtagggcacatggatttcgaaaaacccgacttgaaacggtttccttgtctgagattggcttatgaagccatcaagtctggtggaattatgccaacggtattgaacgcagccaatgaaattgctgtcgaagcgtttttaaatgaagaagtcaaattcactgacatcgcggtcatcatcgagcgcagcatggcccagtttaaaccggacgatgccggcagcctcgaattggttttgcaggccgatcaagatgcgcgcgaggtggctagagacatcatcaagaccttggtagct

SEQ ID NO: 8 - protein, 394 aa, Methylomonas 16a
MKGICILGATGSIGVSTLDVVARHPDKYQVVALTANGNIDALYEQCLAHHPEYAVVVMESKVAEFKQRIAASPVADIKVLSGSEALQQVATLENVDTVMAAIVGAAGLLPTLAAAKAGKTVLLANKEALVMSGQIFMQAVSDSGAVLLPIDSEHNAIFQCMPAGYTPGHTAKQARRILLTASGGPFRRTPIETLSSVTPDQAVAHPKWDMGRKISVDSATMMNKGLELIEACLLFNMEPDQIEVVIHPQSIIHSMVDYVDGSVLAQMGNPDMRTPIAHAMAWPERFDSGVAPLDIFEVGHMDFEKPDLKRFPCLRLAYEAIKSGGIMPTVLNAANEIAVEAFLNEEVKFTDIAVIIERSMAQFKPDDAGSLELVLQADQDAREVARDIIKTLVA

SEQ ID NO: 9 - DNA, 693 bp, Methylomonas 16a
atgaacccaaccatccaatgctgggccgtcgtgcccgcagccggcgtcggcaaacgcatgcaagccgatcgccccaaacaatatttaccgcttgccggtaaaacggtcatcgaacacacactgactcgactacttgagtccgacgccttccaaaaagttgcggtggcgatttccgtcgaagacccttattggcctgaactgtccatagccaaacaccccgacatcatcaccgcgcctggcggcaaggaacgcgccgactcggtgctgtctgcactgaaggctttagaagatatagccagcgaaaatgattgggtgctggtacacgacgccgcccgcccctgcttgacgggcagcgacatccaccttcaaatcgataccttaaaaaatgacccggtcggcggcatcctggccttgagttcgcacgacacattgaaacacgtggatggtgacacgatcaccgcaaccatagacagaaagcacgtctggcgcgccttgacgccgcaaatgttcaaatacggcatgttgcgcgacgcgttgcaacgaaccgaaggcaatccggccgtcaccgacgaagccagtgcgctggaacttttgggccataaacccaaaatcgtggaaggccgcccggacaacatcaaaatcacccgcccggaagatttggccctggcacaattttatatggagcaacaagca

SEQ ID NO: 10 - protein, 231 aa, Methylomonas 16a
MNPTIQCWAVVPAAGVGKRMQADRPKQYLPLAGKTVIEHTLTRLLESDAFQKVAVAISVEDPYWPELSIAKHPDIITAPGGKERADSVLSALKALEDIASENDWVLVHDAARPCLTGSDIHLQIDTLKNDPVGGILALSSHDTLKHVDGDTITATIDRKHVWRALTPQMFKYGMLRDALQRTEGNPAVTDEASALELLGHKPKIVEGRPDNIKITRPEDLALAQFYMEQQA

SEQ ID NO: 11 - DNA, 855 bp, Methylomonas 16a
atggattatgcggctgggtggggcgaaagatggcctgctccggcaaaattgaacttaatgttgaggattaccggtcgcaggccagatggctatcatctgttgcaaacggtgtttcaaatgctcgatctatgcgattggttgacgtttcatccggttgatgatggccgcgtgacgctgcgaaatccaatctccggcgttccagagcaggatgacttgactgttcgggcggctaatttgttgaagtctcataccggctgtgtgcgcggagtttgtatcgatatcgagaaaaatctgcctatgggtggtggtttgggtggtggaagttccgatgctgctacaaccttggtagttctaaatcggctttggggcttgggcttgtcgaagcgtgagttgatggatttgggcttgaggcttggtgccgatgtgcctgtgtttgtgtttggttgttcggcctggggcgaaggtgtgagcgaggatttgcaggcaataacgttgccggaacaatggtttgtcatcattaaaccggattgccatgtgaatactggagaaattttttctgcagaaaatttgacaaggaatagtgcagtcgttacaatgagcgactttcttgcaggggataatcggaatgattgttcggaagtggtttgcaagttatatcgaccggtgaaagatgcaatcgatgcgttgttatgctatgcggaagcgagattgacggggaccggtgcatgtgtgttcgctcagttttgtaacaaggaagatgctgagagtgcgttagaaggattgaaagatcggtggctggtgttcttggctaaaggcttgaatcagtctgcgctctacaagaaattagaacaggga

SEQ ID NO: 12 - protein, 285 aa, Methylomonas 16a
MDYAAGWGERWPAPAKLNLMLRITGRRPDGYHLLQTVFQMLDLCDWLTFHPVDDGRVTLRNPISGVPEQDDLTVRAANLLKSHTGCVRGVCIDIEKNLPMGGGLGGGSSDAATTLVVLNRLWGLGLSKRELMDLGLRLGADVPVFVFGCSAWGEGVSEDLQAITLPEQWFVIIKPDCHVNTGEIFSAENLTRNSAVVTMSDFLAGDNRNDCSEVVCKLYRPVKDAIDALLCYAEARLTGTGACVFAQFCNKEDAESALEGLKDRWLVFLAKGLNQSALYKKLEQG

SEQ ID NO: 13 - DNA, 471 bp, Methylomonas 16a
atgatacgcgtaggcatgggttacgacgtgcaccgtttcaacgacggcgaccacatcattttgggcggcgtcaaaatcccttatgaaaaaggcctggaagcccattccgacggcgacgtggtgctgcacgcattggccgacgccatcttgggagccgccgctttgggcgacatcggcaaacatttcccggacaccgaccccaatttcaagggcgccgacagcagggtgctactgcgccacgtgtacggcatcgtcaaggaaaaaggctataaactggtcaacgccgacgtgaccatcatcgctcaggcgccgaagatgctgccacacgtgcccggcatgcgcgccaacattgccgccgatctggaaaccgatgtcgatttcattaatgtaaaagccacgacgaccgagaaactgggctttgagggccgtaaggaaggcatcgccgtgcaggctgtggtgttgatagaacgc

SEQ ID NO: 14 - protein, 157 aa, Methylomonas 16a
MIRVGMGYDVHRFNDGDHIILGGVKIPYEKGLEAHSDGDVVLHALADAILGAAALGDIGKHFPDTDPNFKGADSRVLLRHVYGIVKEKGYKLVNADVTIIAQAPKMLPHVPGMRANIAADLETDVDFINVKATTTEKLGFEGRKEGIAVQAVVLIER

SEQ ID NO: 15 - DNA, 1632 bp, Methylomonas 16a
atgacaaaattcatctttatcaccggcggcgtggtgtcatccttgggaaaagggatagccgcctcctccctggcggcgattctggaagaccgcggcctcaaagtcactatcacaaaactcgatccctacatcaacgtcgaccccggcaccatgagcccgtttcaacacggcgaggtgttcgtgaccgaagacggtgccgaaaccgatttggaccttggccattacgaacggtttttgaaaaccacgatgaccaagaaaaacaacttcaccaccggtcaggtttacgagcaggtattacgcaacgagcgcaaaggtgattatcttggcgcgaccgtgcaagtcattccacatatcaccgacgaaatcaaacgccgggtgtatgaaagcgccgaagggaaagatgtggcattgatcgaagtcggcggcacggtgggcgacatcgaatcgttaccgtttctggaaaccatacgccagatgggcgtggaactgggtcgtgaccgcgccttgttcattcatttgacgctggtgccttacatcaaatcggccggcgaactgaaaaccaagcccacccagcattcggtcaaagaactgcgcaccatcgggattcagccggacattttgatctgtcgttcagaacaaccgatcccggccagtgaacgccgcaagatcgcgctatttaccaatgtcgccgaaaaggcggtgatttccgcgatcgatgccgacaccatttaccgcattccgctattgctgcgcgaacaaggcctggacgacctggtggtcgatcagttgcgcctggacgtaccagcggcggatttatcggcctgggaaaaggtcgtcgatggcctgactcatccgaccgacgaagtcagcattgcgatcgtcggtaaatatgtcgaccacaccgatgcctacaaatcgctgaatgaagccctgattcatgccggcattcacacgcgccacaaggtgcaaatcagctacatcgactccgaaaccatagaagccgaaggcaccgccaaattgaaaaacgtcgatgcgatcctggtgccgggtggtttcggcgaacgcggcgtggaaggcaagatttctaccgtgcgttttgcccgcgagaacaaaatcccgtatttgggcatttgcttgggcatgcaatcggcggtaatcgaattcgcccgcaacgtggttggcctggaaggcgcgcacagcaccgaattcctgccgaaatcgccacaccctgtgatcggcttgatcaccgaatggatggacgaagccggcgaactggtcacacgcgacgaagattccgatctgggcggcacgatgcgtctgggcgcgcaaaaatgccgcctgaaggctgattccttggcttttcagttgtatcaaaaagacgtcatcaccgagcgtcaccgccaccgctacgaattcaacaatcaatatttaaaacaactggaagcggccggcatgaaattttccggtaaatcgctggacggccgcctggtggagatcatcgagctacccgaacacccctggttcctggcctgccagttccatcccgaattcacctcgacgccgcgtaacggccacgccctattttcgggcttcgtcgaagcggccgccaaacacaaaacacaaggcacagca

SEQ ID NO: 16 - protein, 544 aa, Methylomonas 16a
MTKFIFITGGVVSSLGKGIAASSLAAILEDRGLKVTITKLDPYINVDPGTMSPFQHGEVFVTEDGAETDLDLGHYERFLKTTMTKKNNFTTGQVYEQVLRNERKGDYLGATVQVIPHITDEIKRRVYESAEGKDVALIEVGGTVGDIESLPFLETIRQMGVELGRDRALFIHLTLVPYIKSAGELKTKPTQHSVKELRTIGIQPDILICRSEQPIPASERRKIALFTNVAEKAVISAIDADTIYRIPLLLREQGLDDLVVDQLRLDVPAADLSAWEKVVDGLTHPTDEVSIAIVGKYVDHTDAYKSLNEALIHAGIHTRHKVQISYIDSETIEAEGTAKLKNVDAILVPGGFGERGVEGKISTVRFARENKIPYLGICLGMQSAVIEFARNVVGLEGAHSTEFLPKSPHPVIGLITEWMDEAGELVTRDEDSDLGGTMRLGAQKCRLKADSLAFQLYQKDVITERHRHRYEFNNQYLKQLEAAGMKFSGKSLDGRLVEIIELPEHPWFLACQFHPEFTSTPRNGHALFSGFVEAAAKHKTQGTA

SEQ ID NO: 17 - DNA, 954 bp, Methylomonas 16a
atgcaaatcgtactcgcaaacccccgtggattctgtgccggcgtggaccgggccattgaaattgtcgatcaagccatcgaagcctttggtgcgccgatttatgtgcggcacgaggtggtgcataaccgcaccgtggtcgatggactgaaacaaaaaggtgcggtgttcatcgaggaactaagcgatgtgccggtgggttcctacttgattttcagcgcgcacggcgtatccaaggaggtgcaacaggaagccgaggagcgccagttgacggtattcgatgcgacttgtccgctggtgaccaaagtgcacatgcaggttgccaagcatgccaaacagggccgagaagtgattttgatcggccacgccggtcatccggaagtggaaggcacgatgggccagtatgaaaaatgcaccgaaggcggcggcatttatctggtcgaaactccggaagacgtacgcaatttgaaagtcaacaatcccaatgatctggcctatgtgacgcagacgaccttgtcgatgaccgacaccaaggtcatggtggatgcgttacgcgaacaatttccgtccattaaggagcaaaaaaaggacgatatttgttacgcgacgcaaaaccgtcaggatgcggtgcatgatctggccaagatttccgacctgattctggttgtcggctctcccaatagttcgaattccaaccgtttgcgtgaaatcgccgtgcaactcggtaaacccgcttatttgatcgatacttaccaggatttgaagcaagattggctggagggaattgaagtagtcggggttaccgcgggcgcttcggcgccggaagtgttggtgcaggaagtgatcgatcaactgaaggcatggggcggcgaaaccacttcggtcagagaaaacagcggcatcgaggaaaaggtagtcttttcgattcccaaggagttgaaaaaacatatgcaagcg

SEQ ID NO: 18 - protein, 318 aa, Methylomonas 16a
MQIVLANPRGFCAGVDRAIEIVDQAIEAFGAPIYVRHEVVHNRTVVDGLKQKGAVFIEELSDVPVGSYLIFSAHGVSKEVQQEAEERQLTVFDATCPLVTKVHMQVAKHAKQGREVILIGHAGHPEVEGTMGQYEKCTEGGGIYLVETPEDVRNLKVNNPNDLAYVTQTTLSMTDTKVMVDALREQFPSIKEQKKDDICYATQNRQDAVHDLAKISDLILVVGSPNSSNSNRLREIAVQLGKPAYLIDTYQDLKQDWLEGIEVVGVTAGASAPEVLVQEVIDQLKAWGGETTSVRENSGIEEKVVFSIPKELKKHMQA

SEQ ID NO: 19 - DNA, 891 bp, Methylomonas 16a
atgagtaaattgaaagcctacctgaccgtctgccaagaacgcgtcgagcgcgcgctggacgcccgtctgcctgccgaaaacatactgccacaaaccttgcatcaggccatgcgctattccgtattgaacggcggcaaacgcacccggcccttgttgacttatgcgaccggtcaggctttgggcttgccggaaaacgtgctggatgcgccggcttgcgcggtagaattcatccatgtgtattcgctgattcacgacgatctgccggccatggacaacgatgatctgcgccgcggcaaaccgacctgtcacaaggcttacgacgaggccaccgccattttggccggcgacgcactgcaggcgctggcctttgaagttctggccaacgaccccggcatcaccgtcgatgccccggctcgcctgaaaatgatcacggctttgacccgcgccagcggctctcaaggcatggtgggcggtcaagccatcgatctcggctccgtcggccgcaaattgacgctgccggaactcgaaaacatgcatatccacaagactggcgccctgatccgcgccagcgtcaatctggcggcattatccaaacccgatctggatacttgcgtcgccaagaaactggatcactatgccaaatgcataggcttgtcgttccaggtcaaagacgacattctcgacatcgaagccgacaccgcgacactcggcaagactcagggcaaggacatcgataacgacaaaccgacctaccctgcgctattgggcatggctggcgccaaacaaaaagcccaggaattgcacgaacaagcagtcgaaagcttaacgggatttggcagcgaagccgacctgctgcgcgaactatcgctttacatcatcgagcgcacgcac

SEQ ID NO: 20 - protein, 297 aa, Methylomonas 16a
MSKLKAYLTVCQERVERALDARLPAENILPQTLHQAMRYSVLNGGKRTRPLLTYATGQALGLPENVLDAPACAVEFIHVYSLIHDDLPAMDNDDLRRGKPTCHKAYDEATAILAGDALQALAFEVLANDPGITVDAPARLKMITALTRASGSQGMVGGQAIDLGSVGRKLTLPELENMHIHKTGALIRASVNLAALSKPDLDTCVAKKLDHYAKCIGLSFQVKDDILDIEADTATLGKTQGKDIDNDKPTYPALLGMAGAKQKAQELHEQAVESLTGFGSEADLLRELSLYIIERTH

SEQ ID NO: 21 - DNA, 1533 bp, Methylomonas 16a
atggccaacaccaaacacatcatcatcgtcggcgcgggtcccggcggactttgcgccggcatgttgctgagccagcgcggcttcaaggtatcgattttcgacaaacatgcagaaatcggcggccgcaaccgcccgatcaacatgaacggctttaccttcgataccggtccgacattcttgttgatgaaaggcgtgctggacgaaatgttcgaactgtgcgagcgccgtagcgaggattatctggaattcctgccgctaagcccgatgtaccgcctgctgtacgacgaccgcgacatcttcgtctattccgaccgcgagaacatgcgcgccgaattgcaacgggtattcgacgaaggcacggacggctacgaacagttcatggaacaggaacgcaaacgcttcaacgcgctgtatccctgcatcacccgcgattattccagcctgaaatcctttttgtcgctggacttgatcaaggccctgccgtggctggcttttccgaaaagcgtgttcaataatctcggccagtatttcaaccaggaaaaaatgcgcctggccttttgctttcagtccaagtatctgggcatgtcgccgtgggaatgcccggcactgtttacgatgctgccctatctggagcacgaatacggcatttatcacgtcaaaggcggcctgaaccgcatcgcggcggcgatggcgcaagtgatcgcggaaaacggcggcgaaattcacttgaacagcgaaatcgagtcgctgatcatcgaaaacggcgctgccaagggcgtcaaattacaacatggcgcggagctgcgcggcgacgaagtcatcatcaacgcggattttgcccacgcgatgacgcatctggtcaaaccgggcgtcttgaaaaaatacaccccggaaaacctgaagcagcgcgagtattcctgttcgaccttcatgctgtatctgggtttggacaagatttacgatctgccgcaccataccatcgtgtttgccaaggattacaccaccaatatccgcaacattttcgacaacaaaaccctgacggacgatttttcgttttacgtgcaaaacgccagcgccagcgacgacagcctagcgccagccggcaaatcggcgctgtacgtgctggtgccgatgcccaacaacgacagcggcctggactggcaggcgcattgccaaaacgtgcgcgaacaggtgttggacacgctgggcgcgcgactgggattgagcgacatcagagcccatatcgaatgcgaaaaaatcatcacgccgcaaacctgggaaacggacgaacacgtttacaagggcgccactttcagtttgtcgcacaagttcagccaaatgctgtactggcggccgcacaaccgtttcgaggaactggccaattgctatctggtcggcggcggcacgcatcccggtagcggtttgccgaccatctacgaatcggcgcggatttcggccaagctgatttcccagaaacatcgggtgaggttcaaggacatagcacacagcgcctggctgaaaaaagccaaagcc

SEQ ID NO: 22 - protein, 511 aa, Methylomonas 16a
MANTKHIIIVGAGPGGLCAGMLLSQRGFKVSIFDKHAEIGGRNRPINMNGFTFDTGPTFLLMKGVLDEMFELCERRSEDYLEFLPLSPMYRLLYDDRDIFVYSDRENMRAELQRVFDEGTDGYEQFMEQERKRFNALYPCITRDYSSLKSFLSLDLIKALPWLAFPKSVFNNLGQYFNQEKMRLAFCFQSKYLGMSPWECPALFTMLPYLEHEYGIYHVKGGLNRIAAAMAQVIAENGGEIHLNSEIESLIIENGAAKGVKLQHGAELRGDEVIINADFAHAMTHLVKPGVLKKYTPENLKQREYSCSTFMLYLGLDKIYDLPHHTIVFAKDYTTNIRNIFDNKTLTDDFSFYVQNASASDDSLAPAGKSALYVLVPMPNNDSGLDWQAHCQNVREQVLDTLGARLGLSDIRAHIECEKIITPQTWETDEHVYKGATFSLSHKFSQMLYWRPHNRFEELANCYLVGGGTHPGSGLPTIYESARISAKLISQKHRVRFKDIAHSAWLKKAKA

SEQ ID NO: 23 - DNA, 1491 bp, Methylomonas 16a
atgaactcaaatgacaaccaacgcgtgatcgtgatcggcgccggcctcggcggcctgtccgccgctatttcgctggccacggccggcttttccgtgcaactcatcgaaaaaaacgacaaggtcggcggcaagctcaacatcatgaccaaagacggctttaccttcgatctggggccgtccattttgacgatgccgcacatctttgaggccttgttcacaggggccggcaaaaacatggccgattacgtgcaaatccagaaagtcgaaccgcactggcgcaatttcttcgaggacggtagcgtgatcgacttgtgcgaagacgccgaaacccagcgccgcgagctggataaacttggccccggcacttacgcgcaattccagcgctttctggactattcgaaaaacctctgcacggaaaccgaagccggttacttcgccaagggcctggacggcttttgggatttactcaagttttacggcccgctccgcagcctgctgagtttcgacgtcttccgcagcatggaccagggcgtgcgccgctttatttccgatcccaagttggtcgaaatcctgaattacttcatcaaatacgtcggctcctcgccttacgatgcgcccgccttgatgaacctgctgccttacattcaatatcattacggcctgtggtacgtgaaaggcggcatgtatggcatggcgcaggccatggaaaaactggccgtggaattgggcgtcgagattcgtttagatgccgaggtgtcggaaatccaaaaacaggacggcagagcctgcgccgtaaagttggcgaacggcgacgtgctgccggccgacatcgtggtgtcgaacatggaagtgattccggcgatggaaaaactgctgcgcagcccggccagcgaactgaaaaaaatgcagcgcttcgagcctagctgttccggcctggtgctgcacttgggcgtggacaggctgtatccgcaactggcgcaccacaatttcttttattccgatcatccgcgcgaacatttcgatgcggtattcaaaagccatcgcctgtcggacgatccgaccatttatctggtcgcgccgtgcaagaccgaccccgcccaggcgccggccggctgcgagatcatcaaaatcctgccccatatcccgcacctcgaccccgacaaactgctgaccgccgaggattattcagccttgcgcgagcgggtgctggtcaaactcgaacgcatgggcctgacggatttacgccaacacatcgtgaccgaagaatactggacgccgctggatattcaggccaaatattattcaaaccagggctcgatttacggcgtggtcgccgaccgcttcaaaaacctgggtttcaaggcacctcaacgcagcagcgaattatccaatctgtatttcgtcggcggcagcgtcaatcccggcggcggcatgccgatggtgacgctgtccgggcaattggtgagggacaagattgtggcggatttgcaa

SEQ ID NO: 24 - protein, 497 aa, Methylomonas 16a
MNSNDNQRVIVIGAGLGGLSAAISLATAGFSVQLIEKNDKVGGKLNIMTKDGFTFDLGPSILTMPHIFEALFTGAGKNMADYVQIQKVEPHWRNFFEDGSVIDLCEDAETQRRELDKLGPGTYAQFQRFLDYSKNLCTETEAGYFAKGLDGFWDLLKFYGPLRSLLSFDVFRSMDQGVRRFISDPKLVEILNYFIKYVGSSPYDAPALMNLLPYIQYHYGLWYVKGGMYGMAQAMEKLAVELGVEIRLDAEVSEIQKQDGRACAVKLANGDVLPADIVVSNMEVIPAMEKLLRSPASELKKMQRFEPSCSGLVLHLGVDRLYPQLAHHNFFYSDHPREHFDAVFKSHRLSDDPTIYLVAPCKTDPAQAPAGCEIIKILPHIPHLDPDKLLTAEDYSALRERVLVKLERMGLTDLRQHIVTEEYWTPLDIQAKYYSNQGSIYGVVADRFKNLGFKAPQRSSELSNLYFVGGSVNPGGGMPMVTLSGQLVRDKIVADLQ

SEQ ID NO: 25 - DNA, 912 bp, Pantoea stewartii
ttgacggtctgcgcaaaaaaacacgttcaccttactggcatttcggctgagcagttgctggctgatatcgatagccgccttgatcagttactgccggttcagggtgagcgggattgtgtgggtgccgcgatgcgtgaaggcacgctggcaccgggcaaacgtattcgtccgatgctgctgttattaacagcgcgcgatcttggctgtgcgatcagtcacgggggattactggatttagcctgcgcggttgaaatggtgcatgctgcctcgctgattctggatgatatgccctgcatggacgatgcgcagatgcgtcgggggcgtcccaccattcacacgcagtacggtgaacatgtggcgattctggcggcggtcgctttactcagcaaagcgtttggggtgattgccgaggctgaaggtctgacgccgatagccaaaactcgcgcggtgtcggagctgtccactgcgattggcatgcagggtctggttcagggccagtttaaggacctctcggaaggcgataaaccccgcagcgccgatgccatactgctaaccaatcagtttaaaaccagcacgctgttttgcgcgtcaacgcaaatggcgtccattgcggccaacgcgtcctgcgaagcgcgtgagaacctgcatcgtttctcgctcgatctcggccaggcctttcagttgcttgacgatcttaccgatggcatgaccgataccggcaaagacatcaatcaggatgcaggtaaatcaacgctggtcaatttattaggctcaggcgcggtcgaagaacgcctgcgacagcatttgcgcctggccagtgaacacctttccgcggcatgccaaaacggccattccaccacccaactttttattcaggcctggtttgacaaaaaactcgctgccgtcagttaa

SEQ ID NO: 26 - protein, 303 aa, Pantoea stewartii
LTVCAKKHVHLTGISAEQLLADIDSRLDQLLPVQGERDCVGAAMREGTLAPGKRIRPMLLLLTARDLGCAISHGGLLDLACAVEMVHAASLILDDMPCMDDAQMRRGRPTIHTQYGEHVAILAAVALLSKAFGVIAEAEGLTPIAKTRAVSELSTAIGMQGLVQGQFKDLSEGDKPRSADAILLTNQFKTSTLFCASTQMASIAANASCEARENLHRFSLDLGQAFQLLDDLTDGMTDTGKDINQDAGKSTLVNLLGSGAVEERLRQHLRLASEHLSAACQNGHSTTQLFIQAWFDKKLAAVS

SEQ ID NO: 27 - DNA, 1296 bp, Pantoea stewartii
atgagccattttgcggtgatcgcaccgccctttttcagccatgttcgcgctctgcaaaaccttgctcaggaattagtggcccgcggtcatcgtgttacgttttttcagcaacatgactgcaaagcgctggtaacgggcagcgatatcggattccagaccgtcggactgcaaacgcatcctcccggttccttatcgcacctgctgcacctggccgcgcacccactcggaccctcgatgttacgactgatcaatgaaatggcacgtaccagcgatatgctttgccgggaactgcccgccgcttttcatgcgttgcagatagagggcgtgatcgttgatcaaatggagccggcaggtgcagtagtcgcagaagcgtcaggtctgccgtttgtttcggtggcctgcgcgctgccgctcaaccgcgaaccgggtttgcctctggcggtgatgcctttcgagtacggcaccagcgatgcggctcgggaacgctataccaccagcgaaaaaatttatgactggctgatgcgacgtcacgatcgtgtgatcgcgcatcatgcatgcagaatgggtttagccccgcgtgaaaaactgcatcattgtttttctccactggcacaaatcagccagttgatccccgaactggattttccccgcaaagcgctgccagactgctttcatgcggttggaccgttacggcaaccccaggggacgccggggtcatcaacttcttattttccgtccccggacaaaccccgtatttttgcctcgctgggcaccctgcagggacatcgttatggcctgttcaggaccatcgccaaagcctgcgaagaggtggatgcgcagttactgttggcacactgtggcggcctctcagccacgcaggcaggtgaactggcccggggcggggacattcaggttgtggattttgccgatcaatccgcagcactttcacaggcacagttgacaatcacacatggtgggatgaatacggtactggacgctattgcttcccgcacaccgctactggcgctgccgctggcatttgatcaacctggcgtggcatcacgaattgtttatcatggcatcggcaagcgtgcgtctcggtttactaccagccatgcgctggcgcggcagattcgatcgctgctgactaacaccgattacccgcagcgtatgacaaaaattcaggccgcattgcgtctggcaggcggcacaccagccgccgccgatattgttgaacaggcgatgcggacctgtcagccagtactcagtgggcaggattatgcaaccgcactatga

SEQ ID NO: 28 - protein, 431 aa, Pantoea stewartii
MSHFAVIAPPFFSHVRALQNLAQELVARGHRVTFFQQHDCKALVTGSDIGFQTVGLQTHPPGSLSHLLHLAAHPLGPSMLRLINEMARTSDMLCRELPAAFHALQIEGVIVDQMEPAGAVVAEASGLPFVSVACALPLNREPGLPLAVMPFEYGTSDAARERYTTSEKIYDWLMRRHDRVIAHHACRMGLAPREKLHHCFSPLAQISQLIPELDFPRKALPDCFHAVGPLRQPQGTPGSSTSYFPSPDKPRIFASLGTLQGHRYGLFRTIAKACEEVDAQLLLAHCGGLSATQAGELARGGDIQVVDFADQSAALSQAQLTITHGGMNTVLDAIASRTPLLALPLAFDQPGVASRIVYHGIGKRASRFTTSHALARQIRSLLTNTDYPQRMTKIQAALRLAGGTPAAADIVEQAMRTCQPVLSGQDYATAL

SEQ ID NO: 29 - DNA, 1149 bp, Pantoea stewartii
atgcaaccgcactatgatctcattctggtcggtgccggtctggctaatggccttatcgcgctccggcttcagcaacagcatccggatatgcggatcttgcttattgaggcgggtcctgaggcgggagggaaccatacctggtcctttcacgaagaggatttaacgctgaatcagcatcgctggatagcgccgcttgtggtccatcactggcccgactaccaggttcgtttcccccaacgccgtcgccatgtgaacagtggctactactgcgtgacctcccggcatttcgccgggatactccggcaacagtttggacaacatttatggctgcataccgcggtttcagccgttcatgctgaatcggtccagttagcggatggccggattattcatgccagtacagtgatcgacggacggggttacacgcctgattctgcactacgcgtaggattccaggcatttatcggtcaggagtggcaactgagcgcgccgcatggtttatcgtcaccgattatcatggatgcgacggtcgatcagcaaaatggctaccgctttgtttataccctgccgctttccgcaaccgcactgctgatcgaagacacacactacattgacaaggctaatcttcaggccgaacgggcgcgtcagaacattcgcgattatgctgcgcgacagggttggccgttacagacgttgctgcgggaagaacagggtgcattgcccattacgttaacgggcgataatcgtcagttttggcaacagcaaccgcaagcctgtagcggattacgcgccgggctgtttcatccgacaaccggctactccctaccgctcgcggtggcgctggccgatcgtctcagcgcgctggatgtgtttacctcttcctctgttcaccagacgattgctcactttgcccagcaacgttggcagcaacaggggtttttccgcatgctgaatcgcatgttgtttttagccggaccggccgagtcacgctggcgtgtgatgcagcgtttctatggcttacccgaggatttgattgcccgcttttatgcgggaaaactcaccgtgaccgatcggctacgcattctgagcggcaagccgcccgttcccgttttcgcggcattgcaggcaattatgacgactcatcgttga

SEQ ID NO: 30 - protein, 382 aa, Pantoea stewartii
MQPHYDLILVGAGLANGLIALRLQQQHPDMRILLIEAGPEAGGNHTWSFHEEDLTLNQHRWIAPLVVHHWPDYQVRFPQRRRHVNSGYYCVTSRHFAGILRQQFGQHLWLHTAVSAVHAESVQLADGRIIHASTVIDGRGYTPDSALRVGFQAFIGQEWQLSAPHGLSSPIIMDATVDQQNGYRFVYTLPLSATALLIEDTHYIDKANLQAERARQNIRDYAARQGWPLQTLLREEQGALPITLTGDNRQFWQQQPQACSGLRAGLFHPTTGYSLPLAVALADRLSALDVFTSSSVHQTIAHFAQQRWQQQGFFRMLNRMLFLAGPAESRWRVMQRFYGLPEDLIARFYAGKLTVTDRLRILSGKPPVPVFAALQAIMTTHR

SEQ ID NO: 31 - DNA, 1479 bp, Pantoea stewartii
atgaaaccaactacggtaattggtgcgggctttggtggcctggcactggcaattcgtttacaggccgcaggtattcctgttttgctgcttgagcagcgcgacaagccgggtggccgggcttatgtttatcaggagcagggctttacttttgatgcaggccctaccgttatcaccgatcccagcgcgattgaagaactgtttgctctggccggtaaacagcttaaggattacgtcgagctgttgccggtcacgccgttttatcgcctgtgctgggagtccggcaaggtcttcaattacgataacgaccaggcccagttagaagcgcagatacagcagtttaatccgcgcgatgttgcgggttatcgagcgttccttgactattcgcgtgccgtattcaatgagggctatctgaagctcggcactgtgccttttttatcgttcaaagacatgcttcgggccgcgccccagttggcaaagctgcaggcatggcgcagcgtttacagtaaagttgccggctacattgaggatgagcatcttcggcaggcgttttcttttcactcgctcttagtgggggggaatccgtttgcaacctcgtccatttatacgctgattcacgcgttagaacgggaatggggcgtctggtttccacgcggtggaaccggtgcgctggtcaatggcatgatcaagctgtttcaggatctgggcggcgaagtcgtgcttaacgcccgggtcagtcatatggaaaccgttggggacaagattcaggccgtgcagttggaagacggcagacggtttgaaacctgcgcggtggcgtcgaacgctgatgttgtacatacctatcgcgatctgctgtctcagcatcccgcagccgctaagcaggcgaaaaaactgcaatccaagcgtatgagtaactcactgtttgtactctattttggtctcaaccatcatcacgatcaactcgcccatcataccgtctgttttgggccacgctaccgtgaactgattcacgaaatttttaaccatgatggtctggctgaggatttttcgctttatttacacgcaccttgtgtcacggatccgtcactggcaccggaagggtgcggcagctattatgtgctggcgcctgttccacacttaggcacggcgaacctcgactgggcggtagaaggaccccgactgcgcgatcgtatttttgactaccttgagcaacattacatgcctggcttgcgaagccagttggtgacgcaccgtatgtttacgccgttcgatttccgcgacgagctcaatgcctggcaaggttcggccttctcggttgaacctattctgacccagagcgcctggttccgaccacataaccgcgataagcacattgataatctttatctggttggcgcaggcacccatcctggcgcgggcattcccggcgtaatcggctcggcgaaggcgacggcaggcttaatgctggaggacctgatttga

SEQ ID NO: 32 - protein, 492 aa, Pantoea stewartii
MKPTTVIGAGFGGLALAIRLQAAGIPVLLLEQRDKPGGRAYVYQEQGFTFDAGPTVITDPSAIEELFALAGKQLKDYVELLPVTPFYRLCWESGKVFNYDNDQAQLEAQIQQFNPRDVAGYRAFLDYSRAVFNEGYLKLGTVPFLSFKDMLRAAPQLAKLQAWRSVYSKVAGYIEDEHLRQAFSFHSLLVGGNPFATSSIYTLIHALEREWGVWFPRGGTGALVNGMIKLFQDLGGEVVLNARVSHMETVGDKIQAVQLEDGRRFETCAVASNADVVHTYRDLLSQHPAAAKQAKKLQSKRMSNSLFVLYFGLNHHHDQLAHHTVCFGPRYRELIHEIFNHDGLAEDFSLYLHAPCVTDPSLAPEGCGSYYVLAPVPHLGTANLDWAVEGPRLRDRIFDYLEQHYMPGLRSQLVTHRMFTPFDFRDELNAWQGSAFSVEPILTQSAWFRPHNRDKHIDNLYLVGAGTHPGAGIPGVIGSAKATAGLMLEDLI

SEQ ID NO: 33 - DNA, 891 bp, Pantoea stewartii
atggcggttggctcgaaaagctttgcgactgcatcgacgcttttcgacgccaaaacccgtcgcagcgtgctgatgctttacgcatggtgccgccactgcgacgacgtcattgacgatcaaacactgggctttcatgccgaccagccctcttcgcagatgcctgagcagcgcctgcagcagcttgaaatgaaaacgcgtcaggcctacgccggttcgcaaatgcacgagcccgcttttgccgcgtttcaggaggtcgcgatggcgcatgatatcgctcccgcctacgcgttcgaccatctggaaggttttgccatggatgtgcgcgaaacgcgctacctgacactggacgatacgctgcgttattgctatcacgtcgccggtgttgtgggcctgatgatggcgcaaattatgggcgttcgcgataacgccacgctcgatcgcgcctgcgatctcgggctggctttccagttgaccaacattgcgcgtgatattgtcgacgatgctcaggtgggccgctgttatctgcctgaaagctggctggaagaggaaggactgacgaaagcgaattatgctgcgccagaaaaccggcaggccttaagccgtatcgccgggcgactggtacgggaagcggaaccctattacgtatcatcaatggccggtctggcacaattacccttacgctcggcctgggccatcgcgacagcgaagcaggtgtaccgtaaaattggcgtgaaagttgaacaggccggtaagcaggcctgggatcatcgccagtccacgtccaccgccgaaaaattaacgcttttgctgacggcatccggtcaggcagttacttcccggatgaagacgtatccaccccgtcctgctcatctctggcagcgcccgatctag

SEQ ID NO: 34 - protein, 296 aa, Pantoea stewartii
MAVGSKSFATASTLFDAKTRRSVLMLYAWCRHCDDVIDDQTLGFHADQPSSQMPEQRLQQLEMKTRQAYAGSQMHEPAFAAFQEVAMAHDIAPAYAFDHLEGFAMDVRETRYLTLDDTLRYCYHVAGVVGLMMAQIMGVRDNATLDRACDLGLAFQLTNIARDIVDDAQVGRCYLPESWLEEEGLTKANYAAPENRQALSRIAGRLVREAEPYYVSSMAGLAQLPLRSAWAIATAKQVYRKIGVKVEQAGKQAWDHRQSTSTAEKLTLLLTASGQAVTSRMKTYPPRPAHLWQRPI

SEQ ID NO: 35 - DNA, 528 bp, Pantoea stewartii
atgttgtggatttggaatgccctgatcgtgtttgtcaccgtggtcggcatggaagtggttgctgcactggcacataaatacatcatgcacggctggggttggggctggcatctttcacatcatgaaccgcgtaaaggcgcatttgaagttaacgatctctatgccgtggtattcgccattgtgtcgattgccctgatttacttcggcagtacaggaatctggccgctccagtggattggtgcaggcatgaccgcttatggtttactgtattttatggtccacgacggactggtacaccagcgctggccgttccgctacataccgcgcaaaggctacctgaaacggttatacatggcccaccgtatgcatcatgctgtaaggggaaaagagggctgcgtgtcctttggttttctgtacgcgccaccgttatctaaacttcaggcgacgctgagagaaaggcatgcggctagatcgggcgctgccagagatgagcaggacggggtggatacgtcttcatccgggaagtaa

SEQ ID NO: 36 - protein, 175 aa, Pantoea stewartii
MLWIWNALIVFVTVVGMEVVAALAHKYIMHGWGWGWHLSHHEPRKGAFEVNDLYAVVFAIVSIALIYFGSTGIWPLQWIGAGMTAYGLLYFMVHDGLVHQRWPFRYIPRKGYLKRLYMAHRMHHAVRGKEGCVSFGFLYAPPLSKLQATLRERHAARSGAARDEQDGVDTSSSGK

SEQ ID NO: 37 - DNA, 1599 bp, Rhodococcus erythropolis AN12
gtgagcgcatttctcgacgccgtcgtcgtcggttccggacacaacgcgctcgtttcggccgcgtatctcgcacgtgagggttggtcggtcgaggttctcgagaaggacacggttctcggcggtgccgtctcgaccgtcgagcgatttcccggatacaaggtggaccgggggtcgtctgcgcacctcatgatccgacacagtggcatcatcgaggaactcggactcggcgcgcacggccttcgctacatcgactgtgacccgtgggcgttcgctccgcccgcccctggcaccgacgggccgggcatcgtgtttcatcgcgacctcgatgcaacctgccagtccatcgaacgagcttgcgggacaaaggacgccgacgcgtaccggcggttcgtcgcggtctggtcggagcgcagccgacacgtgatgaaggcattttccacaccgcccaccggatcgaacctgatcggtgcgttcggaggactggccacagcgcgcggcaacagcgaactgtcgcggcagttcctcgcgccgggcgacgcactgctggacgagtatttcgacagtgaggcactcaaggcagcgttggcgtggttcggcgcccagtccgggcctccgatgtcggaaccgggaaccgctccgatggtcggcttcgcggccctcatgcacgtcctgccgcccgggcgagcagtcggagggagcggcgcactgagtgctgcgttggcatcccggatggctgtcgacggcgccaccgtcgcgctcggtgacggcgtgacgtcgatccgccggaactcgaatcactggaccgtcacaaccgagagcggtcgagaagttcacgctcgcaaggtaatcgcgggttgccacatcctcacgacactcgatctcctgggcaacggaggcttcgaccgaaccacgctcgatcactggcggcggaagatcagggtcggccccggcatcggcgctgtattgcgactggcgacatctgcgctcccgtcctaccgcggcgacgccacgacacgggaaagtacctcgggattgcaattactcgtttccgatcgcgcccacttgcgcactgcacacggcgcagcactggcaggggaactgcctcctcgccctgcggttctcggaatgagtttcagcggaatcgatcccacgatcgccccggccgggcggcatcaggtgacactgtggtcgcagtggcagccgtatcgtctcagcggacatcgcgattgggcgtcggtcgccgaggccgaggccgaccggatcgtcggcgagatggaggcttttgcacccggattcaccgattccgtcctcgaccgcttcattcaaactccccgcgacatcgagtcggaattggggatgatcggcggaaatgtcatgcacgtcgagatgtcactcgatcagatgatgttgtggcgaccgcttcccgaactgtccggccatcgcgttccgggagcagacgggttgtatctgaccggagcctcgacgcatcccggtggtggtgtgtccggagccagtggtcgcagtgccgctcgaatcgcactgtccgacagccgccggggtaaagcgagtcagtggatgcgtcgttcgagcaggtcgtga

SEQ ID NO: 38 - protein, 532 aa, Rhodococcus erythropolis AN12
MSAFLDAVVVGSGHNALVSAAYLAREGWSVEVLEKDTVLGGAVSTVERFPGYKVDRGSSAHLMIRHSGIIEELGLGAHGLRYIDCDPWAFAPPAPGTDGPGIVFHRDLDATCQSIERACGTKDADAYRRFVAVWSERSRHVMKAFSTPPTGSNLIGAFGGLATARGNSELSRQFLAPGDALLDEYFDSEALKAALAWFGAQSGPPMSEPGTAPMVGFAALMHVLPPGRAVGGSGALSAALASRMAVDGATVALGDGVTSIRRNSNHWTVTTESGREVHARKVIAGCHILTTLDLLGNGGFDRTTLDHWRRKIRVGPGIGAVLRLATSALPSYRGDATTRESTSGLQLLVSDRAHLRTAHGAALAGELPPRPAVLGMSFSGIDPTIAPAGRHQVTLWSQWQPYRLSGHRDWASVAEAEADRIVGEMEAFAPGFTDSVLDRFIQTPRDIESELGMIGGNVMHVEMSLDQMMLWRPLPELSGHRVPGADGLYLTGASTHPGGGVSGASGRSAARIALSDSRRGKASQWMRRSSRS

SEQ ID NO: 39 - DNA, 30 bp, Methylomonas 16a
ccgagtactgaagcgggtttttgcagggag

SEQ ID NO: 40 - DNA, 25 bp, Methylomonas 16a
gggctagctgctccgattgttacag

SEQ ID NO: 41 - DNA, 38 bp, Artificial Sequence
  primer, derived from Rhodococcus erythropolis AN12
agcagctagcggaggaataaaccatgagcgcatttctc

SEQ ID NO: 42 - DNA, 26 bp, Artificial Sequence
  primer, derived from Rhodococcus erythropolis AN12
gactagtcacgacctgctcgaacgac

SEQ ID NO: 43 - DNA, 25 bp, Artificial Sequence
  primer
atgacggtctgcgcaaaaaaacacg

SEQ ID NO: 44 - DNA, 28 bp, Artificial Sequence
  primer
gagaaattatgttgtggatttggaatgc

SEQ ID NO: 45 - DNA, 19 bp, Artificial Sequence
  primer
gagtttgatcctggctcag

SEQ ID NO: 46 - DNA, 16 bp, Artificial Sequence
  primer
taccttgttacgactt

SEQ ID NO: 47 - DNA, 17 bp, Artificial Sequence
  primer
gtgccagcagymgcggt

SEQ ID NO: 48 - DNA, 21 bp, Artificial Sequence
  primer
atgagcgcatttctcgacgcc

SEQ ID NO: 49 - DNA, 20 bp, Artificial Sequence
  primer
tcacgacctgctcgaacgac

SEQ ID NO: 50 - DNA, 50 bp, Artificial Sequence
  primer
gagaattggctgaaaaaccaaataaataacaaaatttagcgagtaaatgg

SEQ ID NO: 51 - DNA, 50 bp, Artificial Sequence
  primer
ttcaattgacaggggggctcgttctgatttagagttgctgccagcttttt

SEQ ID NO: 52 - DNA, 50 bp, Artificial Sequence
  primer
gggttgtccagatgttggtgagcggtccttataactataactgtaacaat

SEQ ID NO: 53 - DNA, 50 bp, Artificial Sequence
  primer
ttaatggtcttgccatgagatgtgctccgattgttacagttatagttata

SEQ ID NO: 54 - DNA, 50 bp, Artificial Sequence
  primer
ccccctgtcaattgaaagcccgccatttactcgctaaattttgttattta

SEQ ID NO: 55 - DNA, 22 bp, Artificial Sequence
  primer
aaggatccgcgtattcgtactc

SEQ ID NO: 56 - DNA, 40 bp, Artificial Sequence
  primer
ctggatccgatctagaaataggctcgagttgtcgttcagg

SEQ ID NO: 57 - DNA, 30 bp, Artificial Sequence
  primer
aaggatcctactcgagctgacatcagtgct

SEQ ID NO: 58 - DNA, 22 bp, Artificial Sequence
  primer
gctctagatgcaaccagaatcg

SEQ ID NO: 59 - DNA, 24 bp, Artificial Sequence
  primer
tggctcgagagtaaaacactcaag

SEQ ID NO: 60 - DNA, 19 bp, Artificial Sequence
  primer
tagctcgagtcacgcttgc

(19) United States
(12) Patent Application Publication
(10) Pub. No.: US 2004/0077068 A1
(43) Pub. Date: Apr. 22, 2004

(54) Carotenoid production from a single carbon substrate

(76) Inventors: Patricia C. Brzostowicz, West Chester, PA (US); Qiong Cheng, Wilmington, DE (US); Deana J. Dicosimo, Rockland, DE (US); Mattheos Koffas, Williamsville, NY (US); Edward S. Miller, Wilmington, DE (US); James Martin Odom, Kennett Square, PA (US); Stephen K. Picataggio, Wilmington, DE (US); Pierre E. Rouviere, Wilmington, DE (US)

Correspondence Address: S Neil Feltham, E I Du Pont De Nemours and Company, Legal-Patents, Wilmington, DE, 19898, US

(21) Appl. No.: 10/363,567

(22) PCT Filed: Sep. 4, 2001
(86) PCT No.: PCT/US01/27420

(51) Int. Cl. C12N 1/20

(52) U.S. Cl. 435/252.3

(57) ABSTRACT

A method for the production of carotenoid compounds is disclosed. The method relies on the use of microorganisms which metabolize single carbon substrates for the production of carotenoid compounds in high yields.

This application claims the benefit of U.S. Provisional Application No. 60/229,907, filed Sep. 1, 2000 and the benefit of U.S. Provisional Application No. 60/229,858 filed Sep. 1, 2000.

### FIELD OF THE INVENTION

The invention relates to the field of molecular biology and microbiology. More specifically, the invention describes the production of carotenoid compounds from microorganisms which metabolize single carbon substrates as a sole carbon source.

### BACKGROUND OF THE INVENTION

Carotenoids represent one of the most widely distributed and structurally diverse classes of natural pigments, producing pigment colors of light yellow to orange to deep red. Eye-catching examples of carotenogenic tissues include carrots, tomatoes, red peppers, and the petals of daffodils and marigolds. Carotenoids are synthesized by all photosynthetic organisms, as well as some bacteria and fungi. These pigments have important functions in photosynthesis, nutrition, and protection against photooxidative damage. For example, animals do not have the ability to synthesize carotenoids but must instead obtain these nutritionally important compounds through their dietary sources. Structurally, carotenoids are 40-carbon (C₄₀) terpenoids derived from the isoprene biosynthetic pathway and its five-carbon universal isoprene building block, isopentenyl pyrophosphate (IPP). This biosynthetic pathway can be divided into two portions: the upper isoprene pathway, which leads to the formation of IPP, and the lower carotenoid biosynthetic pathway, which converts IPP into long C₃₀ and C₄₀ carotenogenic compounds. Both portions of this pathway are shown in FIG. 1.

Various other crt genes are known, which enable the intramolecular conversion of long C₃₀ and C₄₀ compounds to produce numerous other carotenoid compounds. It is the degree of the carbon backbone's unsaturation, conjugation and isomerization which determines the specific carotenoids unique absorption characteristics and colors. Several reviews discuss the genetics of carotenoid pigment biosynthesis, such as those of Armstrong (J. Bact. 176: 4795-4802 (1994); Annu. Rev. Microbiol. 51:629-659 (1997)).

In reference to the availability of carotenoid genes, public domain databases such as GenBank contain sequences isolated from numerous organisms. For example, there are currently 26 GenBank Accession numbers relating to various crtE genes isolated from 19 different organisms. The less frequently encountered crtZ gene boasts 6 GenBank Accession numbers with each gene isolated from a different organism. A similarly wide selection of carotenoid genes is available for each of the genes discussed above.

The genetics of carotenoid pigment biosynthesis has been extremely well studied in the Gram-negative, pigmented bacteria of the genera Pantoea, formerly known as Erwinia. In both E. herbicola EHO-10 (ATCC 39368) and E. uredovora 20D3 (ATCC 19321), the crt genes are clustered in two genetic units, crt Z and crt EXYIB (U.S. Pat. No. 5,656,472; U.S. Pat. No. 5,5545,816; U.S. Pat. No. 5,530,189; U.S. Pat. No. 5,530,188; U.S. Pat. No. 5,429,939). Despite the similarity in operon structure, the DNA sequences of E. uredovora and E. herbicola show no homology by DNA-DNA hybridization (U.S. Pat. No. 5,429,939).

Although more than 600 different carotenoids have been identified in nature, only a few are used industrially for food colors, animal feeding, pharmaceuticals and cosmetics. Presently, most of the carotenoids used for industrial purposes are produced by chemical synthesis; however, these compounds are very difficult to make chemically (Nelis and Leenheer, Appl. Bacteriol. 70:181-191 (1991)). Natural carotenoids can either be obtained by extraction of plant material or by microbial synthesis. At the present time, only a few plants are widely used for commercial carotenoid production. However, the productivity of carotenoid synthesis in these plants is relatively low and the resulting carotenoids are very expensive.

A number of carotenoids have been produced from microbial sources. For example, Lycopene has been produced from genetically engineered E. coli and Candia utilis (Farmer W. R. and J. C. Liao. (2001) Biotechnol. Prog. 17: 57-61; Wang C. et al., (2000) Biotechnol Prog. 16: 922-926; Misawa, N. and H. Shimada. (1998). J. Biotechnol. 59: 169-181; Shimada, H. et al. 1998. Appl. Environm. Microbiol. 64:2676-2680). β-carotene has been produced from E. coli, Candia utilis and Pfaffia rhodozyma (Albrecht, M. et al., (1999). Biotechnol. Lett. 21: 791-795; Miura, Y. et al., 1998. Appl. Environm. Microbiol. 64:1226-1229; U.S. Pat. No. 5,691,190). Zeaxanthin has been produced from recombinant from E. coli and Candia utilis (Albrecht, M. et al., (1999). Biotechnol. Lett. 21: 791-795; Miura, Y. et al., 1998. Appl. Environm. Microbiol. 64:1226-1229). Astaxanthin has been produced from E. coli and Pfaffia rhodozyma (U.S. Pat. No. 5,466,599; U.S. Pat. No. 6,015,684; U.S. Pat. No. 5,182,208; U.S. Pat. No. 5,972,642).

Additionally genes encoding various elements of the carotenoid biosynthetic pathway have been cloned and expressed in various microbes. For example genes encoding lycopene cyclase, geranylgeranyl pyrophosphate synthase, and phytoene dehydrogenase isolated from Erwinia herbicola have been expressed recombinantly in E. coli (U.S. Pat. No. 5,656,472; U.S. Pat. No. 5,545,816; U.S. Pat. No. 5,530,189; U.S. Pat. No. 5,530,188). Similarly genes encoding the carotenoid products geranylgeranyl pyrophosphate, phytoene, lycopene, β-carotene, and zeaxanthin-diglucoside, isolated from Erwinia uredovora have been expressed in E. coli, Zymomonas mobilis, and Saccharomyces cerevisiae (U.S. Pat. No. 5,429,939). Similarly, the Carotenoid biosynthetic genes crtE (1), crtB (3), crtI (5), crtY (7), and crtZ isolated from Flavobacterium have been recombinantly expressed (U.S. Pat. No. 6,124,113).

Although the above methods of propducing carotenoids are useful, these methods suffer from low yields and reliance on expensive feedstock's. A method that produces higher yields of carotenoids from an inexpensive feedstock is needed.

There are a number of microorganisms that utilize single carbon substrates as sole energy sources. These substrates include, methane, methanol, formate, methylated amines and thiols, and various other reduced carbon compounds which lack any carbon-carbon bonds and are generally quite inexpensive. These organisms are referred to as methylotrophs and herein as “C1 metabolizers”. These organisms are characterized by the ability to use carbon substrates lacking carbon to carbon bonds as a sole source of energy and biomass. A subset of methylotrophs are the methanotrophs which have the unique ability to utilize methane as a sole energy source. Although a large number of these organisms are known, few of these microbes have been successfully harnessed to industrial processes for the synthesis of materials. Although single carbon substrates are cost effective energy sources, difficulty in genetic manipulation of these microorganisms as well as a dearth of information about their genetic machinery has limited their use primarily to the synthesis of native products. For example the commercial applications of biotransformation of methane have historically fallen broadly into three categories: 1) Production of single cell protein, (Sharpe D. H. BioProtein Manufacture 1989. Ellis Horwood series in applied science and industrial technology. New York: Halstead Press.) (Villadsen, John, Recent Trends Chem. React. Eng., [Proc. Int. Chem. React. Eng. Conf.], 2nd (1987), Volume 2, 320-33. Editor(s): Kulkami, B. D.; Mashelkar, R. A.; Sharma, M. M. Publisher: Wiley East., New Delhi, India; Naguib, M., Proc. OAPEC Symp. Petroprotein, [Pap.] (1980), Meeting Date 1979, 253-77 Publisher: Organ. Arab Pet. Exporting Countries, Kuwait, Kuwait.); 2) epoxidation of alkenes for production of chemicals (U.S. Pat. No. 4,348,476); and 3) biodegradation of chlorinated pollutants (Tsien et al., Gas, Oil, Coal, Environ. Biotechnol. 2, [Pap. Int. IGT Symp. Gas, Oil, Coal, Environ. Biotechnol.], 2nd (1990), 83-104. Editor(s): Akin, Cavit; Smith, Jared. Publisher: Inst. Gas Technol., Chicago, Ill.; WO 9633821; Merkley et al., Biorem. Recalcitrant Org., [Pap. Int. In Situ On-Site Bioreclam. Symp.], 3rd (1995), 165-74. Editor(s): Hinchee, Robert E; Anderson, Daniel B.; Hoeppel, Ronald E. Publisher: Battelle Press, Columbus, Ohio.: Meyer et al., Microb. Releases (1993), 2(1), 11-22). Even here, the commercial success of the methane biotransformation has been limited to epoxidation of alkenes due to low product yields, toxicity of products and the large amount of cell mass required to generate product associated with the process.

The commercial utility of methylotrophic organisms is reviewed in Lidstrom and Stirling (Annu. Rev. Microbiol. 44:27-58 (1990)). Little commercial success has been documented, despite numerous efforts involving the application of methylotrophic organisms and their enzymes (Lidstrom and Stirling, supra, Table 3). In most cases, it has been discovered that the organisms have little advantage over other well-developed host systems. Methanol is frequently cited as a feedstock which should provide both economic and quality advantages over other more traditional carbohydrate raw materials, but thus far this expectation has not been significantly validated in published works.

One of the most common classes of single carbon metabolizers are the methanotrophs. Methanotrophic bacteria are defined by their ability to use methane as a sole source of carbon and energy. Methane monooxygenase is the enzyme required for the primary step in methane activation and the product of this reaction is methanol (Murrell et al., Arch. Microbiol. (2000), 173(5-6), 325-332). This reaction occurs at ambient temperature and pressures whereas chemical transformation of methane to methanol requires temperatures of hundreds of degrees and high pressure (Grigoryan, E. A., Kinet. Catal. (1999), 40(3), 350-363; WO 2000007718; U.S. Pat. No. 5,750,821). It is this ability to transform methane under ambient conditions along with the abundance of methane that makes the biotransformation of methane a potentially unique and valuable process.

Many methanotrophs contain an inherent isoprenoid pathway which enables these organisms to synthesize other non-endogenous isoprenoid compounds. Since methanotrophs can use one carbon substrate (methane or methanol) as an energy source, it is possible to produce carotenoids at low cost.

Current knowledge in the field concerning methylotrophic organisms and carotenoids leads to the following conclusions. First, there is tremendous commercial incentive arising from abundantly available C1 sources, which could be used as a feedstock for C1 organisms and which should provide both economic and quality advantages over other more traditional carbohydrate raw materials. Secondly, there is abundant knowledge available concerning organisms that possess carotenogenic biosynthetic genes, the function of those genes, and the upper isoprene pathway which produces carotenogenic precursor molecules. Finally, numerous methylotrophic organisms exist in the art which are themselves pigmented, and thereby possess portions of the necessary carotenoid biosynthetic pathway.

Despite these available tools, the art does not reveal any C1 metabolizers which have been genetically engineered to make specific carotenoids of choice, for large scale commercial value. It is hypothesized that the usefulness of these organisms for production of a larger range of chemicals is constrained by limitations including, relatively slow growth rates of methanotrophs, limited ability to tolerate methanol as an alternative substrate to methane, difficulty in genetic engineering, poor understanding of the roles of multiple carbon assimilation pathways present in methanotrophs, and potentially high costs due to the oxygen demand of fully saturated substrates such as methane. The problem to be solved, therefore is to provide a cost effective method for the microbial production of carotenoid compounds, using organisms which utilize C1 compounds as their carbon and energy source.

Applicants have solved the stated problem by engineering microorganisms which are able to use single carbon substrates as sole carbon sources for the production of carotenoid compounds.

### SUMMARY OF THE INVENTION

The invention provides a method for the production of a carotenoid compound comprising:

(a) providing a transformed C1 metabolizing host cell comprising:

(i) suitable levels of isopentenyl pyrophosphate; and

(ii) at least one isolated nucleic acid molecule encoding an enzyme in the carotenoid biosynthetic pathway under the control of suitable regulatory sequences;

(b) contacting the host cell of step (a) under suitable growth conditions with an effective amount of a C1 carbon substrate whereby an carotenoid compound is produced.

Preferred C1 carbon substrates of the invention are selected from the group consisting of methane, methanol; formaldehyde, formic acid, methylated amines, methylated thiols, and carbon dioxide. Preferred C1 metabolizers are methylotrophs and methanotrophs. Particularly preferred C1 metabolizers are those that comprise a functional Embden-Meyerhof carbon pathway, said pathway comprising a gene encoding a pyrophosphate dependent phosphofructokinase enzyme. Optionally the preferred host may comprise at least one gene encoding a fructose bisphosphate aldolase enzyme.

Suitable levels of isopentenyl pyrophosphate may be endogenous to the host, or may be provided by heterologusly introduced upper pathway isoprenoid genes such as D-1-deoxyxylulose-5-phosphate synthase (Dxs), D-1-deoxyxylulose-5-phosphate reductoisomerase (Dxr), 2C-methyl-d-erythritol cytidylyltransferase (IspD), 4-diphosphocytidyl-2-C-methylerythritol kinase (IspE), 2C-methyl-d-erythritol 2,4-cyclodiphosphate synthase (IspF), CTP synthase (PyrG) and lytB.

In an alternate embodiment the invention provides a method for the over-production of carotenoid production in a transformed C1 metabolizing host comprising:

(a) providing a transformed C1 metabolizing host cell comprising:

(i) suitable levels of isopentenyl pyrophosphate; and

(ii) at least one isolated nucleic acid molecule encoding an enzyme in the carotenoid biosynthetic pathway under the control of suitable regulatory sequences; and

(iii) either:

1) multiple copies of at least one gene encoding an enzyme selected from the group consisting of D-1-deoxyxylulose-5-phosphate synthase (Dxs), D-1-deoxyxylulose-5-phosphate reductoisomerase (Dxr), 2C-methyl-d-erythritol cytidylyltransferase (IspD), 4-diphosphocytidyl-2-C-methylerythritol kinase (IspE), 2C-methyl-d-erythritol 2,4-cyclodiphosphate synthase (IspF), CTP synthase (PyrG) and lytB; or

2) at least one gene encoding an enzyme selected from the group consisting of D-1-deoxyxylulose-5-phosphate synthase (Dxs), D-1-deoxyxylulose-5-phosphate reductoisomerase (Dxr), 2C-methyl-d-erythritol cytidylyltransferase (IspD), 4-diphosphocytidyl-2-C-methylerythritol kinase (IspE), 2C-methyl-d-erythritol 2,4-cyclodiphosphate synthase (IspF), CTP synthase (PyrG) and lytB operable linked to a strong promoter.

(b) contacting the host cell of step (a) under suitable growth conditions with an effective amount of a C1 carbon substrate whereby a carotenoid compound is over-produced.

### BRIEF DESCRIPTION OF THE DRAWINGS, SEQUENCE DESCRIPTIONS AND BIOLOGICAL DEPOSITS

FIG. 1 illustrates the upper isoprene pathway and lower carotenoid biosynthetic pathway.

FIG. 2 provides microarray expression data for key carbon pathway genes, as expressed in Methylomonas 16a.

FIG. 3 shows plasmid pcrt1 and HPLC spectra verifying synthesis of β-carotene in those Methylomonas containing plasmid pcrt1.

FIG. 4 shows plasmid pcrt3 and HPLC spectra verifying synthesis of zeaxanthin and its mono- and di-glucosides in those Methylomonas containing plasmid pcrt3.

FIG. 5 shows plasmid pcrt4 and HPLC spectra verifying synthesis of zeaxanthin and its mono- and di-glucosides in those Methylomonas containing plasmid pcrt4.

FIG. 6 shows plasmid pcrt4.1 and HPLC spectra verifying synthesis of canthaxanthin and astaxanthin in those Methylomonas containing plasmid pcrt4.1.

FIG. 7 shows plasmid pTJS75::dxs:dxr:lacZ:Tn5Kn and production of the native carotenoid in those Methylomonas containing plasmid pTJS75::dxs:dxr:lacZ:Tn5Kn. Additionally, the construct pcrt4.1 is shown.

The invention can be more fully understood from the following detailed description and the accompanying sequence descriptions which form a part of this application.

The following sequences conform with 37 C.F.R. 1.821-1.825 (“Requirements for Patent Applications Containing Nucleotide Sequences and/or Amino Acid Sequence Disclosures—the Sequence Rules”) and consistent with World Intellectual Property Organization (WIPO) Standard ST.25 (1998) and the sequence listing requirements of the EPO and PCT (Rules 5.2 and 49.5(a-bis), and Section 208 and Annex C of the Administrative Instructions). The symbols and format used for nucleotide and amino acid sequence data comply with the rules set forth in 37 C.F.R. §1.822.

SEQ ID NOs:1-38 are full length genes or proteins as identified in Table 1.

TABLE 1
Summary of Gene and Protein SEQ ID Numbers

| | SEQ ID | |
|---|---|---|
| | Nucleic | SEQ ID |
| Description | acid | Peptide |
| Phosphofructokinase pyrophosphate | 1 | 2 |
| dependent | | |
| KHG/KDPG Aldolase | 3 | 4 |
| dxs | 5 | 6 |
| dxr | 7 | 8 |
| ispD (ygbP | 9 | 10 |
| ispE(ychB) | 11 | 12 |
| ispF (ygbB) | 13 | 14 |
| pyrG | 15 | 16 |
| lytB | 17 | 18 |
| ispA | 19 | 20 |
| CrtN1 | 21 | 22 |
| CrtN2 | 23 | 24 |
| crtE | 25 | 26 |
| crtX | 27 | 28 |
| crtY | 29 | 30 |
| crtI | 31 | 32 |
| crtB | 33 | 34 |
| crtZ | 35 | 36 |
| crtO | 37 | 38 |

SEQ ID Nos:39-40 are amplification primers for the HMPS promoter

SEQ ID Nos:41-42 are amplification primers for the crtO gene from Rhodococcus.

SEQ ID NOs:43 and 44 are the primer sequences used to amplify the crt cluster of Pantoea stewartii.

SEQ ID NOs:45-47 are the primer sequences used to amplify the 16s rRNA of Rhodococcus erythropolis AN12.

SEQ ID NOs:48 and 49 are the primer sequences used to amplify the crtO gene.

SEQ ID NOs: 50-54 are promoter sequences for the HMPS gene and primers used to amplify that promoter.

SEQ ID NOs:55 and 56 are the primer sequences used to amplify the dxs gene.

SEQ ID NOs:57 and 58 are the primer sequences used to amplify the dxr gene.

SEQ ID NOs:59 and 60 are the primer sequences used to amplify the lytB gene.

Applicants made the following biological deposits under the terms of the Budapest Treaty on the International Recognition of the Deposit of Micro-organisms for the Purposes of Patent Procedure:

| | International | |
|---|---|---|
| Depositor Identification | Depository | |
| Reference | Designation | Date of Deposit |
| Methylomonas 16a | ATCC PTA 2402 | Aug. 22, 2000 |

### DETAILED DESCRIPTION OF THE INVENTION

The present method is useful for the creation of recombinant organisms that have the ability to produce various carotenoid compounds. Nucleic acid fragments encoding a variety of enzymes implicated in the carotenoid biosynthetic pathway have been cloned into microorganisms which use single carbon substrates as a sole carbon source for the production of carotenoid compounds.

There is a general practical utility for microbial production of carotenoid compounds as these compounds are very difficult to make chemically (Nelis and Leenheer, Appl. Bacteriol. 70:181-191 (1991)). Most carotenoids have strong color and can be viewed as natural pigments or colorants. Furthermore, many carotenoids have potent antioxidant properties and thus inclusion of these compounds in the diet is thought to be healthful. Well-known examples are β-carotene and astaxanthin. Additionally, carotenoids are required elements of aquaculture. Salmon and shrimp aquaculture are particularly useful applications for this invention as carotenoid pigmentation is critically important for the value of these organisms. (F. Shahidi, J. A. Brown, Carotenoid pigments in seafood and aquaculture: Critical reviews in food Science 38(1): 1-67 (1998)). Finally, carotenoids have utility as intermediates in the synthesis of steroids, flavors and fragrances and compounds with potential electro-optic applications.

The disclosure below provides a detailed description of the selection of the appropriate C1 metabolizing microorganism for transformation and the production of various carotenoid compounds in high yield.

In this disclosure, a number of terms and abbreviations are used. The following definitions are provided.

The term “Embden-Meyerhof pathway” refers to the series of biochemical reactions for conversion of hexoses such as glucose and fructose to important cellular 3-carbon intermediates such as glyceraldehyde-3-phosphate, dihydroxyacetone phosphate, phosphophenol pyruvate and pyruvate. These reactions typically proceed with net yield of biochemically useful energy in the form of ATP. The key enzymes unique to the Embden-Meyerhof pathway are the phosphofructokinase and fructose-1,6 bisphosphate aldolase.

The term “Entner-Douderoff pathway” refers to a series of biochemical reactions for conversion of hexoses such as glucose or fructose to the important 3-carbon cellular intermediates pyruvate and glyceraldehyde-3-phosphate without any net production of biochemically useful energy. The key enzymes unique to the Entner-Douderoff pathway are the 6-phosphogluconate dehydratase and a ketodeoxyphosphogluconate aldolase.

The term “diagnostic” as it relates to the presence of a gene in a pathway refers to evidence of the presence of that pathway, where a gene having that activity is identified. Within the context of the present invention the presence of a gene encoding a pyrophosphate dependant phosphofructokinase is “diagnostic” for the presence of the Embden-Meyerhof carbon pathway and the presence of gene encoding a ketodeoxyphosphogluconate aldolase is “diagnostic” for the presence of the Entner-Douderoff carbon pathway.

The term “yield” is defined herein as the amount of cell mass produced per gram of carbon substrate metabolized.

The term “carbon conversion efficiency” is a measure of how much carbon is assimilated into cell mass and is calculated assuming a biomass composition of CH₂O_{0.5}N_{0.25}.

The term “C₁ carbon substrate” refers to any carbon-containing molecule that lacks a carbon-carbon bond. Examples are methane, methanol, formaldehyde, formic acid, formate, methylated amines (e.g., mono-, di-, and tri-methyl amine), methylated thiols, and carbon dioxide.

The term “C1 metabolizer” refers to a microorganism that has the ability to use an single carbon substrate as a sole source of energy and biomass. C1 metabolizers will typically be methylotrophs and/or methanotrophs.

The term “methylotroph” means an organism capable of oxidizing organic compounds which do not contain carbon-carbon bonds. Where the methylotroph is able to oxidize CH4, the methylotroph is also a methanotroph.

The term “methanotroph” means a prokaryote capable of utilizing methane as a substrate. Complete oxidation of methane to carbon dioxide occurs by aerobic degradation pathways. Typical examples of methanotrophs useful in the present invention include but are not limited to the genera Methylomonas, Methylobacter, Methylococcus, and Methylosinus.

The term “high growth methanotrophic bacterial strain” refers to a bacterium capable of growth with methane or methanol as sole carbon and energy source which possess a functional Embden-Meyerhof carbon flux pathway resulting in a yield of cell mass per gram of C1 substrate metabolized. The specific “high growth methanotrophic bacterial strain” described herein is referred to as “Methylomonas 16a” or “16a”, which terms are used interchangeably.

The term “Methylomonas 16a” and “Methylomonas 16a sp.” Are used interchangeably and refer to the Methylomonas strain used in the present invention.

The term “isoprenoid compound” refers to any compound which is derived via the pathway beginning with isopentenyl pyrophosphate (IPP) and formed by the head-to-tail condensation of isoprene units which may be of 5, 10, 15, 20, 30 or 40 carbons in length. There term “isoprenoid pigment” refers to a class of isoprenoid compounds which typically have strong light absorbing properties.

The term “upper isoprene pathway” refers to any of the following genes and gene products associated with the isoprenoid biosynthetic pathway including the dxs gene (encoding 1-deoxyxylulose-5-phosphate synthase), the dxr gene (encoding 1-deoxyxylulose-5-phosphate reductoisomerase), the “ispD” gene (encoding the 2C-methyl-D-erythritol cytidyltransferase enzyme; also known as ygbP), the “ispE” gene (encoding the 4-diphosphocytidyl-2-C-methylerythritol kinase; also known as ychB), the “ispF” gene (encoding a 2C-methyl-d-erythritol 2,4-cyclodiphosphate synthase; also known as ygbB), the “pyrG” gene (encoding a CTP synthase); the “lytB” gene involved in the formation of dimethylallyl diphosphate; and the gcpE gene involved in the synthesis of 2-C-methyl-D-erythritol 4-phosphate in the isoprenoid pathway.

The term “Dxs” refers to the 1-deoxyxylulose-5-phosphate synthase enzyme encoded by the dxs gene.

The term “Dxr” refers to the 1-deoxyxylulose-5-phosphate reductoisomerase enzyme encoded by the dxr gene.

The term “YgbP” or “IspD” refers to the 2C-methyl-D-erythritol cytidyltransferase enzyme encoded by the ygbP or ispD gene. The names of the gene, ygbP or ispD, are used interchangeably in this application. The names of gene product, YgbP or IspD are used interchangeably in this application.

The term “YchB” or “IspE” refers to the 4-diphosphocytidyl-2-C-methylerythritol kinase enzyme encoded by the ychB or ispE gene. The names of the gene, ychB or ispE, are used interchangeably in this application. The names of gene product, YchB or IspE are used interchangeably in this application.

The term “YgbB” or “IspF” refers to the 2C-methyl-d-erythritol 2,4-cyclodiphosphate synthase enzyme encoded by the ygbB or ispF gene. The names of the gene, ygbB or ispF, are used interchangeably in this application. The names of gene product, YgbB or IspF are used interchangeably in this application.

The term “PyrG” refers to a CTP synthase enzyme encoded by the pyrG gene.

The term “IspA” refers to Geranyltransferase or farnesyl diphosphate synthase enzyme as one of prenyl transferase family encoded by ispA gene.

The term “LytB” refers to protein having a role in the formation of dimethylallyl-pyrophosphate in the isoprenoid pathway and which is encoded by lytB gene.

The term “gcpE” refers to a protein having a role in the formation of 2-C-methyl-D-erythritol 4-phosphate in the isoprenoid pathway (Altincicek et al., J. Bacteriol. (2001), 183(8), 2411-2416; Campos et al., FEBS Lett. (2001), 488(3), 170-173)

The term “lower carotenoid biosynthetic pathway” refers to any of the following genes and gene products associated with the isoprenoid biosynthetic pathway, which are involved in the immediate synthesis of phytoene (whose synthesis represents the first step unique to biosynthesis of carotenoids) or subsequent reactions. These genes and gene products include the “ispA” gene (encoding geranyltransferase or farnesyl diphosphate synthase), the “ctrN” and “ctrN 1” genes (encoding diapophytoene dehydrogenases), the “crtE” gene (encoding geranylgeranyl pyrophosphate synthase), the “crtX” gene (encoding zeaxanthin glucosyl transferase), the “crtY” gene (encoding lycopene cyclase), the “crtI” gene (encoding phytoene desaturase), the “crtB” gene (encoding phytoene synthase), the “crtZ” gene (encoding β-carotene hydroxylase), and the “crtO” gene (encoding a β-carotene ketolase). Additionally, the term “carotenoid biosynthetic enzyme” is an inclusive term referring to any and all of the enzymes in the present pathway including CrtE, CrtX, CrtY, CrtI, CrtB, CrtZ, and CrtO.

The term “IspA” refers to the protein encoded by the ispA gene, and whose activity catalyzes a sequence of 3 prenyltransferase reactions in which geranyl pyrophosphate (GPP), farnesyl pyrophosphate (FPP), and geranylgeranyl pyrophosphate (GGPP) are formed.

The term “CrtN1” or “CrtN, copy1” refers to copy 1 of the diapophytoene dehydrogenase enzyme encoded by crtN1 gene.

The term “CrtN2” or “CrtN copy2” refers to copy 2 of the diapophytoene dehydrogenase enzyme(Crt) encoded by crtN2 gene.

The term “CrtE” refers to geranylgeranyl pyrophosphate synthase enzyme encoded by crtE gene which converts trans-trans-farnesyl diphosphate and isopentenyl diphosphate into pyrophosphate and geranylgeranyl diphosphate

The term “CrtX” refers to the zeaxanthin glucosyl transferase enzyme encoded by the crtX (gene, and which glycosolates zeaxanthin to produce zeaxanthin-β-diglucoside.

The term “CrtY” refers to the lycopene cyclase enzyme encoded by the crtY gene and which catalyzes conversion of lycopene to β-carotene.

The term “CrtI” refers to the phytoene desaturase enzyme encoded by the crtI gene and which converts phytoene into lycopene via the intermediaries of phytofluene, zeta-carotene, and neurosporene by the introduction of 4 double bonds.

The term “CrtB” refers to the phytoene synthase enzyme encoded by the crtB gene which catalyses the reaction from prephytoene diphosphate to phytoene.

The term “CrtZ” refers to the β-carotene hydroxylase enzyme encoded by crtZ gene which catalyses the hydroxylation reaction from β-carotene to zeaxanthin.

The term “CrtO” refers to the β-carotene ketolase enzyme encoded by crtO gene which catalyses conversion of β-carotene into canthaxanthin (two ketone groups) via echinenone (one ketone group) as the intermediate.

The term “Carotenoid compound” is defined as a class of hydrocarbons (carotenes) and their oxygenated derivatives (xanthophylls) consisting of eight isoprenoid units joined in such a manner that the arrangement of isoprenoid units is reversed at the center of the molecule so that the two central methyl groups are in a 1,6-positional relationship and the remaining nonterminal methyl groups are in a 1,5-positional relationship. All carotenoids may be formally derived from the acyclic C40H56 structure (Formula I below), having a long central chain of conjugated double bonds, by (i) hydrogenation, (ii) dehydrogenation, (iii) cyclization, or (iv) oxidation, or any combination of these processes.

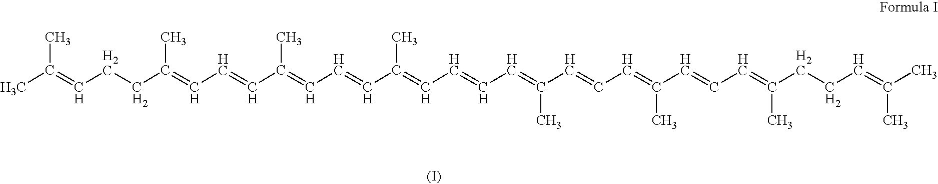

This class also includes certain compounds that arise from certain rearrangements of the carbon skeleton (I), or by the (formal) removal of part of this structure.

For convenience carotenoid formulae are often written in a shorthand form as

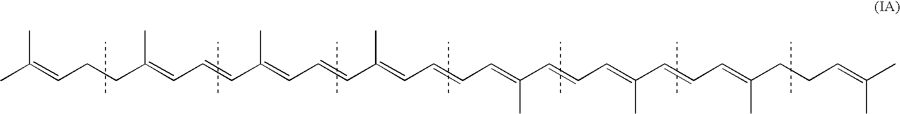

where the broken lines indicate formal division into isoprenoid units.

As used herein, an “isolated nucleic acid fragment” is a polymer of RNA or DNA that is single- or double-stranded, optionally containing synthetic, non-natural or altered nucleotide bases. An isolated nucleic acid fragment in the form of a polymer of DNA may be comprised of one or more segments of cDNA, genomic DNA or synthetic DNA.

“Gene” refers to a nucleic acid fragment that is capable of being expressed as a specific protein, including regulatory sequences preceding (5′ non-coding sequences) and following (3′ non-coding sequences) the coding sequence. “Native gene” refers to a gene as found in nature with its own regulatory sequences. “Chimeric gene” refers to any gene that is not a native gene, comprising regulatory and coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. “Endogenous gene” refers to a native gene in its natural location in the genome of an organism. A “foreign” gene refers to a gene not normally found in the host organism, but that is introduced into the host organism by gene transfer. Foreign genes can comprise native genes inserted into a non-native organism, or chimeric genes. A “transgene” is a gene that has been introduced into the genome by a transformation procedure.

“Coding sequence” refers to a DNA sequence that codes for a specific amino acid sequence. “Suitable regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, introns, polyadenylation recognition sequences, RNA processing site, effector binding site and stem-loop structure.

“Promoter” refers to a DNA sequence capable of controlling the expression of a coding sequence or functional RNA. In general, a coding sequence is located 3′ to a promoter sequence. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental or physiological conditions. Promoters which cause a gene to be expressed in most cell types at most times are commonly referred to as “constitutive promoters”. It is further recognized that since in most cases the exact boundaries of regulatory sequences have not been completely defined, DNA fragments of different lengths may have identical promoter activity.

The term “operably linked” refers to the association of nucleic acid sequences on a single nucleic acid fragment so that the function of one is affected by the other. For example, a promoter is operably linked with a coding sequence when it is capable of affecting the expression of that coding sequence (i.e., that the coding sequence is under the transcriptional control of the promoter). Coding sequences can be operably linked to regulatory sequences in sense or antisense orientation.

The term “expression”, as used herein, refers to the transcription and stable accumulation of sense (mRNA) or antisense RNA derived from the nucleic acid fragment of the invention. Expression may also refer to translation of mRNA into a polypeptide.

“Transformation” refers to the transfer of a nucleic acid fragment into the genome of a host organism, resulting in genetically stable inheritance. Host organisms containing the transformed nucleic acid fragments are referred to as “transgenic” or “recombinant” or “transformed” organisms.

The terms “plasmid”, “vector” and “cassette” refer to an extra chromosomal element often carrying genes which are not part of the central metabolism of the cell, and usually in the form of circular double-stranded DNA fragments. Such elements may be autonomously replicating sequences, genome integrating sequences, phage or nucleotide sequences, linear or circular, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell. “Transformation cassette” refers to a specific vector containing a foreign gene and having elements in addition to the foreign gene that facilitates transformation of a particular host cell. “Expression cassette” refers to a specific vector containing a foreign gene and having elements in addition to the foreign gene that allow for enhanced expression of that gene in a foreign host.

The term “percent identity”, as known in the art, is a relationship between two or more polypeptide sequences or two or more polynucleotide sequences, as determined by comparing the sequences. In the art, “identity” also means the degree of sequence relatedness between polypeptide or polynucleotide sequences, as the case may be, as determined by the match between strings of such sequences. “Identity” and “similarity” can be readily calculated by known methods, including but not limited to those described in: Computational Molecular Biology (Lesk, A. M., ed.) Oxford University Press, NY (1988); Biocomputing: Informatics and Genome Projects (Smith, D. W., ed.) Academic Press, NY (1993); Computer Analysis of Sequence Data, Part I (Griffin, A. M., and Griffin, H. G., eds.) Humana Press, NJ (1994); Sequence Analysis in Molecular Biology (von Heinje, G., ed.) Academic Press (1987); and Sequence Analysis Primer (Gribskov, M. and Devereux, J., eds.) Stockton Press, NY (1991). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR Inc., Madison, Wis.). Multiple alignment of the sequences was performed using the Clustal method of alignment (Higgins and Sharp (1989) CABIOS. 5:151-153) with the default parameters (GAP PENALTY=10, GAP LENGTH PENALTY=10). Default parameters for pairwise alignments using the Clustal method were KTUPLE 1, GAP PENALTY=3, WINDOW=5 and DIAGONALS SAVED=5.

Suitable nucleic acid fragments (isolated polynucleotides of the present invention) encode polypeptides that are at least about 70% identical, preferably at least about 80% identical to the amino acid sequences reported herein. Preferred nucleic acid fragments encode amino acid sequences that are about 85% identical to the amino acid sequences reported herein. More preferred nucleic acid fragments encode amino acid sequences that are at least about 90% identical to the amino acid sequences reported herein. Most preferred are nucleic acid fragments that encode amino acid sequences that are at least about 95% identical to the amino acid sequences reported herein. Suitable nucleic acid fragments not only have the above homologies but typically encode a polypeptide having at least 50 amino acids, preferably at least 100 amino acids, more preferably at least 150 amino acids, still more preferably at least 200 amino acids, and most preferably at least 250 amino acids.

A nucleic acid molecule is “hybridizable” to another nucleic acid molecule, such as a cDNA, genomic DNA, or RNA, when a single stranded form of the nucleic acid molecule can anneal to the other nucleic acid molecule under the appropriate conditions of temperature and solution ionic strength. Hybridization and washing conditions are well known and exemplified in Sambrook, J., Fritsch, E. F. and Maniatis, T. Molecular Cloning: A Laboratory Manual, Second Edition, Cold Spring Harbor Laboratory Press, Cold Spring Harbor (1989), particularly Chapter 11 and Table 11.1 therein (entirely incorporated herein by reference). The conditions of temperature and ionic strength determine the “stringency” of the hybridization. Stringency conditions can be adjusted to screen for moderately similar fragments, such as homologous sequences from distantly related organisms, to highly similar fragments, such as genes that duplicate functional enzymes from closely related organisms. Post-hybridization washes determine stringency conditions. One set of preferred conditions uses a series of washes starting with 6×SSC, 0.5% SDS at room temperature for 15 min, then repeated with 2×SSC, 0.5% SDS at 45° C. for 30 min, and then repeated twice with 0.2×SSC, 0.5% SDS at 50° C. for 30 min. A more preferred set of stringent conditions uses higher temperatures in which the washes are identical to those above except for the temperature of the final two 30 min washes in 0.2×SSC, 0.5% SDS was increased to 60° C. Another preferred set of highly stringent conditions uses two final washes in 0.1×SSC, 0.1% SDS at 65° C. An additional preferred set of stringent conditions include 0.1×SSC, 0.1.% SDS, 65° C. and washed with 2×SSC, 0.1% SDS followed by 0.1×SSC, 0.1% SDS).

Hybridization requires that the two nucleic acids contain complementary sequences, although depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the greater the value of Tm for hybrids of nucleic acids having those sequences. The relative stability (corresponding to higher Tm) of nucleic acid hybridizations decreases in the following order: RNA:RNA, DNA:RNA, DNA:DNA. For hybrids of greater than 100 nucleotides in length, equations for calculating Tm have been derived (see Sambrook et al., supra, 9.50-9.51). For hybridizations with shorter nucleic acids, i.e., oligonucleotides, the position of mismatches becomes more important, and the length of the oligonucleotide determines its specificity (see Sambrook et al., supra, 11.7-11.8). In one embodiment the length for a hybridizable nucleic acid is at least about 10 nucleotides. Preferable a minimum length for a hybridizable nucleic acid is at least about 15 nucleotides; more preferably at least about 20 nucleotides; and most preferably the length is at least 30 nucleotides. Furthermore, the skilled artisan will recognize that the temperature and wash solution salt concentration may be adjusted as necessary according to factors such as length of the probe.

The term “sequence analysis software” refers to any computer algorithm or software program that is useful for the analysis of nucleotide or amino acid sequences. “Sequence analysis software” may be commercially available or independently developed. Typical sequence analysis software will include but is not limited to the GCG suite of programs (Wisconsin Package Version 9.0, Genetics Computer Group (GCG), Madison, Wis.), BLASTP, BLASTN, BLASTX (Altschul et al., J. Mol. Biol. 215:403410 (1990), and DNASTAR (DNASTAR, Inc. 1228 S. Park St. Madison, Wis. 53715 USA), and the FASTA program incorporating the Smith-Waterman algorithm (W. R. Pearson, Comput. Methods Genome Res., [Proc. Int. Symp.] (1994), Meeting Date 1992, 111-20. Editor(s): Suhai, Sandor. Publisher: Plenum, New York, N.Y.). Within the context of this application it will be understood that where sequence analysis software is used for analysis, that the results of the analysis will be based on the “default values” of the program referenced, unless otherwise specified. As used herein “default values” will mean any set of values or parameters which originally load with the software when first initialized.

Standard recombinant DNA and molecular cloning techniques used here are well known in the art and are described by Sambrook, J., Fritsch, E. F. and Maniatis, T., Molecular Cloning: A Laboratory Manual, Second Edition, Cold Spring Harbor Laboratory Press, Cold Spring Harbor, N.Y. (1989) (hereinafter “Maniatis”); and by Silhavy, T. J., Bennan, M. L. and Enquist, L. W., Experiments with Gene Fusions, Cold Spring Harbor Laboratory Cold Press Spring Harbor, N.Y. (1984); and by Ausubel, F. M. et al., Current Protocols in Molecular Biology, published by Greene Publishing Assoc. and Wiley-Interscience (1987).

Identification and Isolation of C1 Metabolizing Microorganisms

The present invention provides for the expression of genes involved in the biosynthesis of carotenoid compounds in microorganisms which are able to use single carbon substrates as a sole energy source. Such microorganisms are referred to herein as C1 metabolizers. The host microorganism may be any C1 metabolizer which has the ability to synthesize isopentenyl pyrophosphate (IPP) the precursor for many of the carotenoids.

Many C1 metabolizing microorganisms are known in the art which are able to use a variety of single carbon substrates. Single carbon substrates useful in the present invention include but are not limited to methane, methanol, formaldehyde, formic acid, methylated amines (e.g. mono-, di- and tri-methyl amine), methylated thiols, and carbon dioxide.

All C1 metabolizing microorganisms are generally classified as methylotrophs. Methylotrophs may be defined as any organism capable of oxidizing organic compounds which do not contain carbon-carbon bonds. A subset of methylotrophs are the methanotrophs, which have the distinctive ability to oxidize methane. Facultative methylotrophs have the ability to oxidize organic compounds which do not contain carbon-carbon bonds, but may also use other carbon substrates such as sugars and complex carbohydrates for energy and biomass. Obligate methylotrophs are those organisms which are limited to the use of organic compounds which do not contain carbon-carbon bonds for the generation of energy and obligate methanotrophs are those obligate methylotrophs that have the ability to oxidize methane.

Facultative methylotrophic bacteria are found in many environments, but are isolated most commonly from soil, landfill and waste treatment sites. Many facultative methylotrophs are members of the β, and γ subgroups of the Proteobacteria (Hanson et al., Microb. Growth C1 Compounds., [Int. Symp.], 7th (1993), 285-302. Editor(s): Murrell, J. Collin; Kelly, Don P. Publisher: Intercept, Andover, UK; Madigan et al., Brock Biology of Microorganisms, 8th edition, Prentice Hall, UpperSaddle River, N.J. (1997)). Facultative methylotrophic bacteria suitable in the present invention include but are not limited to, Methylophilus, Methylobacillus, Methylobacterium, Hyphomicrobium, Xanthobacter, Bacillus, Paracoccus, Nocardia, Arthrobacter, Rhodopseudomonas, and Pseudomonas.

The ability to utilize single carbon substrates is not limited to bacteria but extends also to yeasts and fungi. A number of yeast genera are able to use single carbon substrates in addition to more complex materials as energy sources. Specific methylotrophic yeasts useful in the present invention include but are not limited to Candida, Hansenula, Pichia, Torulopsis, and Rhodotorula.

Those methylotrophs having the additional ability to utilize methane are referred to as methanotrophs. Of particular interest in the present invention are those obligate methanotrophs which are methane utilizers but which are obliged to use organic compounds lacking carbon-carbon bonds. Exemplary of these organisms are included in, but not limited to, the genera Methylomonas, Methylobacter, Mehtylococcus, Methylosinus, Methylocyctis, Methylomicrobium, and Methanomonas.

Of particular interest in the present invention are high growth obligate methanotrophs having an energetically favorable carbon flux pathway. For example, Applicants have discovered a specific strain of methanotroph having several pathway features which make it particularly useful for carbon flux manipulation. This type of strain has served as the host in the present application and is known as Methylomonas 16a (ATCC PTA 2402).

The present strain contains several anomalies in the carbon utilization pathway. For example, based on genome sequence data, the strain is shown to contain genes for two pathways of hexose metabolism. The Entner-Douderoff Pathway, which utilizes the keto-deoxy phosphogluconate aldolase enzyme, is present in the strain. It is generally well accepted that this is the operative pathway in obligate methanotrophs. Also present however is the Embden-Meyerhof Pathway, which utilizes the fructose bisphosphate aldolase enzyme. It is well known that this pathway is either not present or not operative in obligate methanotrophs. Energetically, the latter pathway is most favorable and allows greater yield of biologically useful energy, which ultimately results in greater yield production of cell mass and other cell mass-dependent products in Methylomonas 16a. The activity of this pathway in the present 16a strain has been confirmed through microarray data and biochemical evidence measuring the reduction of ATP. Although the 16a strain has been shown to possess both the Embden-Meyerhof and the Entner-Douderoff pathway enzymes, the data suggests that the Embden-Meyerhof pathway enzymes are more strongly expressed than the Entner-Douderoff pathway enzymes. This result is surprising and counter to existing beliefs on the glycolytic metabolism of methanotrophic bacteria. Applicants have discovered other methanotrophic bacteria having this characteristic, including for example, Methylomonas clara and Methylosinus sporium. It is likely that this activity has remained undiscovered in methanotrophs due to the lack of activity of the enzyme with ATP, the typical phosphoryl donor for the enzyme in most bacterial systems.

A particularly novel and useful feature of the Embden-Meyerhof pathway in strain 16a is that the key phosphofructokinase step is pyrophosphate dependent instead of ATP dependent. This feature adds to the energy yield of the pathway by using pyrophosphate instead of ATP. Because of its significance in providing an energetic advantage to the strain, this gene in the carbon flux pathway is considered diagnostic for the present strain.

Comparison of the pyrophosphate dependent phosphofructokinase gene sequence (SEQ ID NO:1) and deduced amino acid sequence (SEQ ID NO:2) to public databases reveals that the most similar known sequence is about 63% identical to the amino acid sequence of reported herein over length of 437 amino acids using a Smith-Waterman alignment algorithm (W. R. Pearson, Comput. Methods Genome Res., [Proc. Int. Symp.] (1994), Meeting Date 1992, 111-20. Editor(s): Suhai, Sandor. Publisher: Plenum, New York, N.Y.). More preferred amino acid fragments are at least about 80%-90% identical to the sequences herein. Most preferred are nucleic acid fragments that are at least 95% identical to the amino acid fragments reported herein. Similarly, preferred pyrophosphate dependent phosphofructokinase encoding nucleic acid sequences corresponding to the instant gene are those encoding active proteins and which are at least 80% identical to the nucleic acid sequences of reported herein. More preferred pyrophosphate dependent phosphofructokinase nucleic acid fragments are at least 90% identical to the sequences herein. Most preferred are pyrophosphate dependent phosphofructokinase nucleic acid fragments that are at least 95% identical to the nucleic acid fragments reported herein.

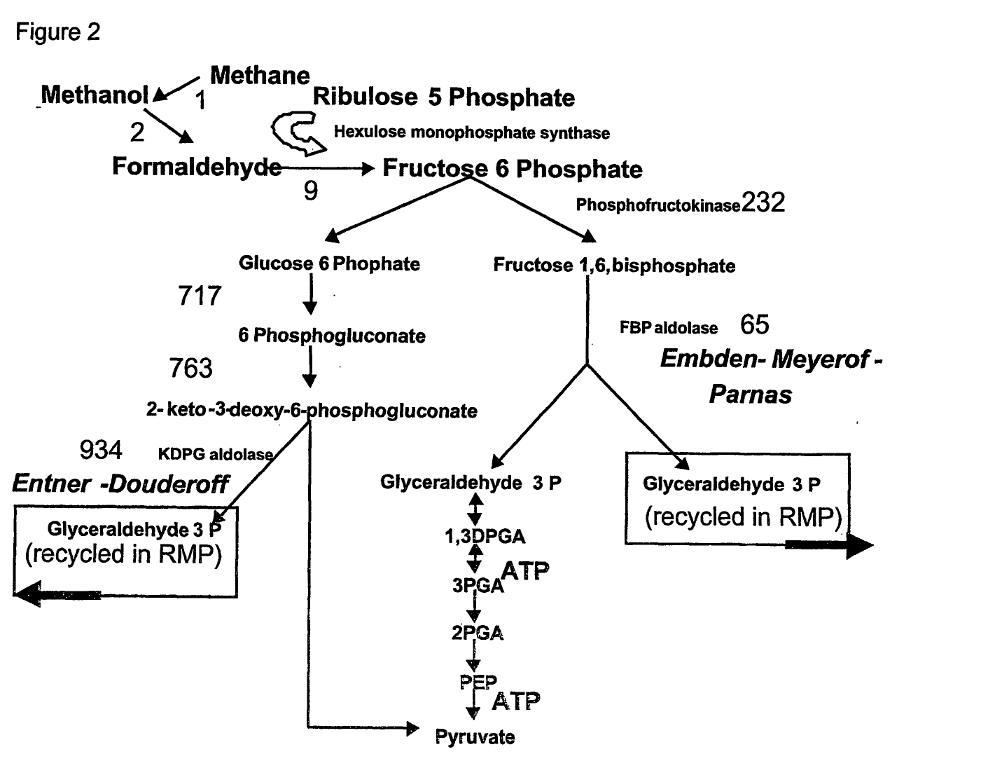

A further distinguishing characteristic of the present strain is revealed when examining the “cleavage” step which occurs in the Ribulose Monophosphate Pathway, or RuMP cycle. This cyclic set of reactions converts methane to biomolecules in methanotrophic bacteria. The pathway is comprised of three phases, each phase being a series of enzymatic steps (FIG. 2). The first step is “fixation” or incorporation of C-1 (formaldehyde) into a pentose to form a hexose or six-carbon sugar. This occurs via a condensation reaction between a 5-carbon sugar (pentose) and formaldehyde and is catalyzed by the hexulose monophosphate synthase enzyme. The second phase is termed “cleavage” and results in splitting of that hexose into two 3-carbon molecules. One of those three-carbon molecules is recycled back through the RuMP pathway, while the other 3-carbon fragment is utilized for cell growth. In methanotrophs and methylotrophs, the RuMP pathway may occur as one of three variants. However, only two of these variants are commonly found, identified as the FBP/TA (fructose bisphosphotase/transaldolase) pathway or the KDPG/TA (keto deoxy phosphogluconate/transaldolase) pathway (Dijkhuizen L., G. E. Devries. The Physiology and biochemistry of aerobic methanol-utilizing gram negative and gram positive bacteria. In: Methane and Methanol Utilizers (1992), eds. Colin Murrell and Howard Dalton; Plenum Press:NY).

The present strain is unique in the way it handles the “cleavage” steps as genes were found that carry out this conversion via fructose bisphosphate as a key intermediate. The genes for fructose bisphosphate aldolase and transaldolase were found clustered together on one piece of DNA. Secondly, the genes for the other variant involving the keto deoxy phosphogluconate intermediate were also found clustered together. Available literature teaches that these organisms (methylotrophs and methanotrophs) rely solely on the KDPG pathway and that the FBP-dependent fixation pathway is utilized by facultative methylotrophs (Dijkhuizen et al., supra). Therefore the latter observation is expected whereas the former is not. The finding of the FBP genes in an obligate methane utilizing bacterium is both surprising and suggestive of utility. The FBP pathway is energetically favorable to the host microorganism due to the fact that less energy (ATP) is utilized than is utilized in the KDPG pathway. Thus organisms that utilize the FBP pathway may have an energetic advantage and growth advantage over those that utilize the KDPG pathway. This advantage may also be useful for energy-requiring production pathways in the strain. By using this pathway a methane-utilizing bacterium may have an advantage over other methane utilizing organisms as production platforms for either single cell protein or for any other product derived from the flow of carbon through the RuMP pathway.

Accordingly the present invention provides a method for the production of a carotenoid compound comprising providing a transformed C1 metabolizing host cell which

(a) grows on a C1 carbon substrate selected from the group consisting of methane and methanol; and

(b) comprises a functional Embden-Meyerhof carbon pathway, said pathway comprising a gene encoding a pyrophosphate dependent phosphofructokinase enzyme.

Isolation of C1 Metabolizing Microorganisms

The C1 metabolizing microorganisms of the present invention are ubiquitous and many have been isolated and characterized. A general scheme for isolation of these strains includes addition of an inoculum into a sealed liquid mineral salts media, containing either methane or methanol. Care must be made of the volume:gas ratio and cultures are typically incubated between 25-55° C. Typically, a variety of different methylotrophic bacteria can be isolated from a first enrichment, if it is plated or streaked onto solid media when growth is first visible. Methods for the isolation of methanotrophs are common and well known in the art (See for example Thomas D. Brock in Biotechnology: A Textbook of Industrial Microbiology, Second Edition (1989) Sinauer Associates, Inc., Sunderland, Mass.; Deshpande, Mukund V., Appl. Biochem. Biotechnol., 36: 227 (1992); or Hanson, R. S. et al. The Prokaryotes: a handbook on habitats, isolation, and identification of bacteria; Springer-Verlag: Berlin, N.Y., 1981; Volume 2, Chapter 118).

As noted above, preferred C1 metabolizer is one that incorporates an active Embden-Meyerhof pathway as indicated by the presence of a pyrophosphate dependent phosphofructokinase. It is contemplated that the present teaching will enable the general identification and isolation of similar strains. For example, the key characteristics of the present high growth strain are that it is an obligate methanotroph, using only either methane of methanol as a sole carbon source and possesses a functional Embden-Meyerhof, and particularly a gene encoding a pyrophosphate dependent phosphofructokinase. Methods for the isolation of methanotrophs are common and well known in the art (See for example Thomas D. Brock supra or Deshpande, supra). Similarly, pyrophosphate dependent phosphofructokinase has been well characterized in mammalian systems and assay methods have been well developed (see for example Schliselfeld et al. Clin. Biochem. (1996), 29(1), 79-83; Clark et al., J. Mol. Cell. Cardiol. (1980), 12(10), 1053-64. The contemporary microbiologist will be able to use these techniques to identify the present high growth strain.

Genes Involved in Carotenoid Production.

The enzyme pathway involved in the biosynthesis of carotenoids can be conveniently viewed in two parts, the upper isoprenoid pathway providing for the conversion of pyruvate and glyceraldehyde-3-phosphate to isopentenyl pyrophosphate and the lower carotenoid biosynthetic pathway, which provides for the synthesis of phytoene and all subsequently produced carotenoids. The upper pathway is ubiquitous in many C1 metabolizing microorganisms and in these cases it will only be necessary to introduce genes that comprise the lower pathway for the biosynthesis of the desired carotenoid. The key division between the two pathways concerns the synthesis of isopentenyl pyrophosphate (IPP). Where IPP is naturally present only elements of the lower carotenoid pathway will be needed. However, it will be appreciated that for the lower pathway carotenoid genes to be effective in the production of carotenoids, it will be necessary for the host cell to have suitable levels of IPP within the cell. Where IPP synthesis is not provided by the host cell, it will be necessary to introduce the genes necessary for the production of IPP. Each of these pathways will be discussed below in detail.

The Upper Isoprenoid Pathway

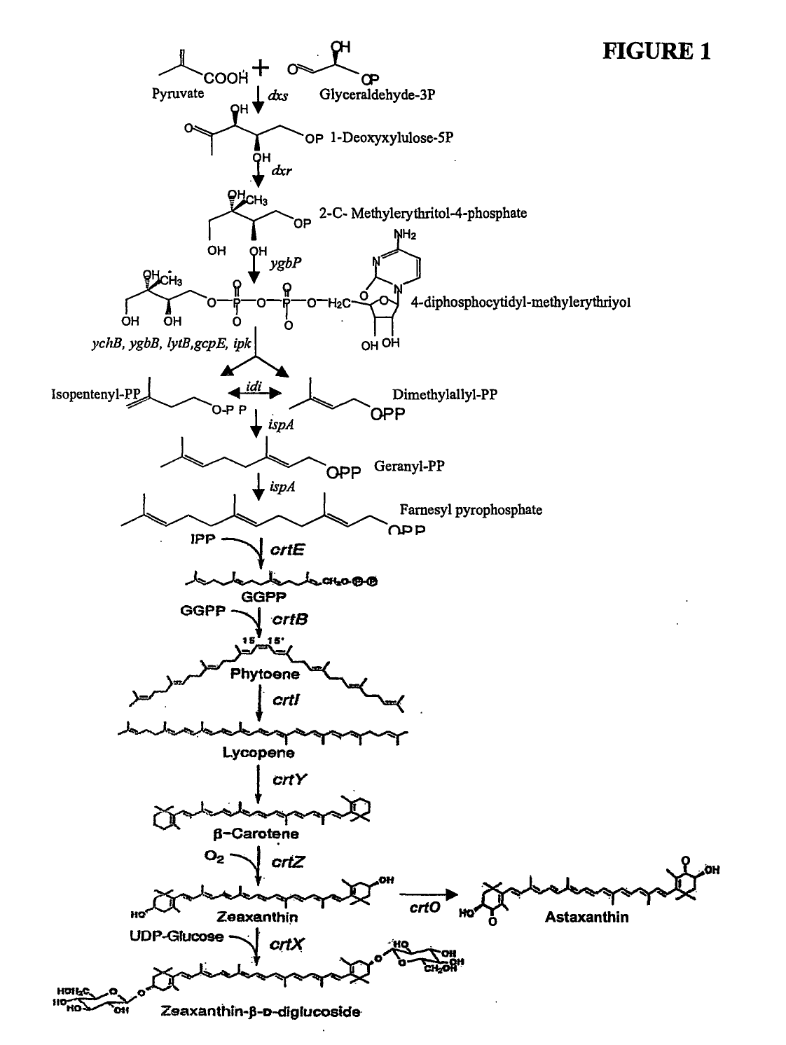

IPP biosynthesis occurs through either of two pathways. First, IPP may be synthesized through the well-known acetate/mevalonate pathway. However, recent studies have demonstrated that the mevalonate-dependent pathway does not operate in all living organisms. An alternate mevalonate-independent pathway for IPP biosynthesis has been characterized in bacteria and in green algae and higher plants (Horbach et al., FEMS Microbiol. Lett. 111:135-140 (1993); Rohmer et al, Biochem. 295: 517-524 (1993); Schwender et al., Biochem. 316: 73-80 (1996); Eisenreich et al., Proc. Natl. Acad. Sci. USA 93: 6431-6436 (1996)). Many steps in both isoprenoid pathways are known (FIG. 1). For example, the initial steps of the alternate pathway leading to the production of IPP have been studied in Mycobacterium tuberculosis by Cole et al. (Nature 393:537-544 (1998)). The first step of the pathway involves the condensation of two 3-carbon molecules (pyruvate and D-glyceraldehyde 3-phosphate) to yield a 5-carbon compound known as D-1-deoxyxylulose-5-phosphate. This reaction occurs by the DXS enzyme, encoded by the dxs gene. Next, the isomerization and reduction of D-1-deoxyxylulose-5-phosphate yields 2-C-methyl-D-erythritol-4-phosphate. One of the enzymes involved in the isomerization and reduction process is D-1-deoxyxylulose-5-phosphate reductoisomerase (DXR), encoded by the gene dxr. 2-C-methyl-D-erythritol-4-phosphate is subsequently converted into 4-diphosphocytidyl-2C-methyl-D-erythritol in a CTP-dependent reaction by the enzyme encoded by the non-annotated gene ygbP (Cole et al., supra). Recently, however, the ygbP gene was renamed as ispD as a part of the isp gene cluster (SwissProtein Accession #Q46893).

Next, the 2^{nd} position hydroxy group of 4-diphosphocytidyl-2C-methyl-D-erythritol can be phosphorylated in an ATP-dependent reaction by the enzyme encoded by the ychB gene. This product phosphorylates 4-diphosphocytidyl-2C-methyl-D-erythritol, resulting in 4-diphosphocytidyl-2C-methyl-D-erythritol 2-phosphate. The ychB gene was renamed as ispE, also as a part of the isp gene cluster (SwissProtein Accession #P24209). Finally, the product of ygbB gene converts 4-diphosphocytidyl-2C-methyl-D-erythritol 2-phosphate to 2C-methyl-D-erythritol 2,4-cyclodiphosphate in a CTP-dependent manner. This gene has also been recently renamed, and belongs to the isp gene cluster. Specifically, the new name for the ygbB gene is ispF (SwissProtein Accession #P36663).

It is known that 2C-methyl-D-erythritol 2,4-cyclodiphosphate can be further converted into IPP to ultimately produce carotenoids in the carotenoid biosynthesis pathway. However, the reactions leading to the production of isopentenyl monophosphate from 2C-methyl-D-erythritol 2,4-cyclodiphosphate are not yet well-characterized. The enzymes encoded by the lytB and gcpE genes (and perhaps others) are thought to participate in the reactions leading to formation of isopentenyl pyrophosphate (IPP) and dimethylallyl pyrophosphate (DMAPP).

IPP may be isomerized to DMAPP via IPP isomerase, encoded by the idi gene, however this enzyme is not essential for survival and may be absent in some bacteria using 2-C-methyl-D-erythritol 4-phosphate (MEP) pathway. Recent evidence suggests that the MEP pathway branches before IPP and separately produces IPP and DMAPP via the lytB gene product. A lytB knockout mutation is lethal in E. coli except in media supplemented with both IPP and DMAPP.

Genes encoding elements of the upper pathway are known from a variety of plant, animal, and bacterial sources, as shown in Table 2.

TABLE 2
Sources of Genes Encoding the Upper Isoprene Pathway

| | Genbank Accession Number and |
|---|---|
| Gene | Source Organism |
| dxs | AF035440, Escherichia coli |
| | Y18874, Synechococcus PCC6301 |
| | AB026631, Streptomyces sp. CL190 |
| | AB042821, Streptomyces griseolosporeus |
| | AF111814, Plasmodium falciparum |
| | AF143812, Lycopersicon esculentum |
| | AJ279019, Narcissus pseudonarcissus |
| | AJ291721, Nicotiana tabacum |
| dxr | AB013300, Escherichia coli |
| | AB049187, Streptomyces griseolosporeus |
| | AF111813, Plasmodium falciparum |
| | AF116825, Mentha x piperita |
| | AF148852, Arabidopsis thaliana |
| | AF182287, Artemisia annua |
| | AF250235, Catharanthus roseus |
| | AF282879, Pseudomonas aeruginosa |
| | AJ242588, Arabidopsis thaliana |
| | AJ250714, Zymomonas mobilis strain ZM4 |
| | AJ292312, Klebsiella pneumoniae, |
| | AJ297566, Zea mays |
| ispD | AB037876, Arabidopsis thaliana |
| | AF109075, Clostridium difficile |
| | AF230736, Escherichia coli |
| | AF230737, Arabidopsis thaliana |
| ispE | AF216300, Escherichia coli |
| | AF263101, Lycopersicon esculentum |
| | AF288615, Arabidopsis thaliana |
| ispF | AB038256, Escherichia coil mecs gene |
| | AF230738, Escherichia coli |
| | AF250236, Catharanthus roseus (MECS) |
| | AF279661, Plasmodium falciparum |
| | AF321531, Arabidopsis thaliana |
| pyrG | AB017705, Aspergillus oryzae |
| | AB064659, Aspergillus kawachii |
| | AF061753, Nitrosomonas europaea |
| | AF206163, Solorina crocea |
| | L22971, Spiroplasma citri |
| | M12843, E. coli |
| | M19132, Emericella nidulans |
| | M69112, Mucor circinelloides |
| | U15192, Chlamydia trachomatis |
| | U59237, Synechococcus PCC7942 |
| | U88301, Mycobacterium bovis |
| | X06626, Aspergillus niger |
| | X08037, Penicillium chrysogenum |
| | X53601, P. blakesleeanus |
| | X67216, A. brasilense |
| | Y11303, A. fumigatus |
| | Y13811, Aspergillus oryzae |
| | NM_001905, |
| | Homo sapiens CTP synthase (CTPS), mRNA |
| | NM_016748, Mus musculus cytidine 5′-triphosphate |
| | synthase (Ctps), mRNA |
| | NM_019857 |
| | Homo sapiens CTP synthase II (CTPS2), |
| | X68196 |
| | mRNA S.cerevisiae ura8 gene for CTP synthetase |
| | XM_013134 |
| | BC009408, Homo sapiens, CTP synthase, clone |
| | MGC10396 IMAGE 3355881 |
| | Homo sapiens CTP synthase II (CTPS2), mRNA |
| | XM_046801 |
| | Homo sapiens CTP synthase II (CTPS2), mRNA |
| | XM_046802 |
| | Homo sapiens CTP synthase II (CTPS2), mRNA |
| | XM_046803 |
| | Homo sapiens CTP synthase II (CTPS2), mRNA |
| | XM_046804 |
| | Homo sapiens CTP synthase II (CTPS2), mRNA |
| | Z47198, A. parasiticus pksA gene for polyketide |
| | synthase |
| lytB | AF027189, Acinetobacter sp. BD413 |
| | AF098521, Burkholderia pseudomallei |
| | AF291696, Streptococcus pneumoniae |
| | AF323927, Plasmodium falciparum gene |
| | M87645, Bacillus subtillis |
| | U38915, Synechocystis sp. |
| | X89371, C. jejuni |
| gcpE | sp O67496 |
| | sp P54482 |
| | tr Q9pky3 |
| | tr Q9Z8H0 |
| | sp O84060 |
| | sp P27433 |
| | sp P44667 |
| | tr Q9ZLL0 |
| | sp O33350 |
| | pir S77159 |
| | tr Q9WZZ3 |
| | sp O83460 |
| | tr Q9JZ40 |
| | tr Q9PPMI |
| | tr Q9RXC9 |
| | tr AAG07190 |
| | tr Q9KTX1 |

The most preferred source of genes for the upper isoprene pathway in the present invention is from Methylomonas 16a. Methylomonas 16a is particularly well suited for the present invention, as the methanotroph is naturally pink-pigmented, producing a 30-carbon carotenoid. Thus, the organism is well-endowed with the genes of the upper isoprene pathway. Sequences of these preferred genes are presented as the following SEQ ID numbers: the dxs gene (SEQ ID NO:5), the dxr gene (SEQ ID NO:7), the “ispD” gene (SEQ ID NO:9), the “ispE” gene (SEQ ID NO:11), the “ispF” gene (SEQ ID NO:13), the “pyrG” gene (SEQ ID NO:15), and the “lytB” gene (SEQ ID NO:17).

The Lower Carotenoid Biosynthetic Pathway

The formation of phytoene is the first “true” step unique in the biosynthesis of carotenoids and produced via the lower carotenoid biosynthetic pathway, despite the compound's being colorless. The synthesis of phytoene occurs via isomerization of IPP to dimethylallyl pyrophosphate (DMAPP). This reaction is followed by a sequence of 3 prenyltransferase reactions. Two of these reactions are catalyzed by ispA, leading to the creation of geranyl pyrophosphate (GPP; a 10-carbon molecule) and farnesyl pyrophosphate (FPP; 15-carbon molecule).

The gene crtN1 and N2 convert farnesyl pyrophosphate to naturally occurring 16A 30-carbon pigment.

The gene crtE, encoding GGPP synthetase is responsible for the 3^{rd} prenyltransferase reaction which may occur, leading to the synthesis of phytoene. This reaction adds IPP to FPP to produce a 20-carbon molecule, geranylgeranyl pyrophosphate (GGPP).

Finally, a condensation reaction of two molecules of GGPP occur to form phytoene (PPPP), the first 40-carbon molecule of the lower carotenoid biosynthesis pathway. This enzymatic reaction is catalyzed by crtB, encoding phytoene synthase.

Lycopene, which imparts a “red”-colored spectra, is produced from phytoene through four sequential dehydrogenation reactions by the removal of eight atoms of hydrogen, catalyzed by the gene crtI (encoding phytoene desaturase). Intermediaries in this reaction are phtyofluene, zeta-carotene, and neurosporene.

Lycopene cyclase (crtY) converts lycopene to β-carotene.

β-carotene is converted to zeaxanthin via a hydroxylation reaction resulting from the activity of β-carotene hydroxylase (encoded by the crtZ gene). B-cryptoxanthin is an intermediate in this reaction.

β-carotene is converted to canthaxanthin by β-carotene ketolase encoded by the crtW gene. Echinenone in an intermediate in this reaction. Canthaxanthin can then be converted to astaxanthin by β-carotene hydroxylase encoded by the crtZ gene. Adonbirubrin is an intermediate in this reaction.

Zeaxanthin can be converted to zeaxanthin-β-diglucoside. This reaction is catalyzed by zeaxanthin glucosyl transferase (crtX).

Zeaxanthin can be converted to astaxanthin by β-carotene ketolase encoded by crtW, crtO or bkt. Adonixanthin is an intermediate in this reaction.

Spheroidene can be converted to spheroidenone by spheroidene monooxygenase encoded by crtA.

Nerosporene can be converted spheroidene and lycopene can be converted to spirilloxanthin by the sequential actions of hydroxyneurosporene synthase, methoxyneurosporene desaturase and hydroxyneurosporene-O-methyltransferase encoded by the crtC, crtD and crtF genes, respectively.

β-carotene can be converted to isorenieratene by b-carotene desaturase encoded by crtU.

Genes encoding elements of the lower carotenoid biosynthetic pathway are known from a variety of plant, animal, and bacterial sources, as shown in Table 3.

TABLE 3
Sources of Genes Encoding the Lower Carotenoid
Biosynthetic Pathway

| | Genbank Accession Number and |
|---|---|
| Gene | Source Organism |
| ispA | AB003187, Micrococcus luteus |
| | AB016094, Synechococcus elongatus |
| | AB021747, Oryza sativa FPPS1 gene for farnesyl |
| | diphosphate synthase |
| | AB028044, Rhodobacter sphaeroides |
| | AB028046, Rhodobacter capsulatus |
| | AB028047, Rhodovulum sulfidophilum |
| | AF112881 and AF136602, Artemisia annua |
| | AF384040, Mentha x piperita |
| | D00694, Escherichia coli |
| | D13293, B. stearothermophilus |
| | D85317, Oryza sativa |
| | X75789, A. thaliana |
| | Y12072, G. arboreum |
| | Z49786, H. brasiliensis |
| | U80605, Arabidopsis thaliana farnesyl diphosphate |
| | synthase precursor (FPS1) mRNA, complete cds |
| | X76026, K. lactis FPS gene for farnesyl diphosphate |
| | synthetase, QCR8 gene for bc1 complex, subunit VIII |
| | X82542, P. argentatum mRNA for farnesyl |
| | diphosphate synthase (FPS1) |
| | X82543, P. argentatum mRNA for farnesyl |
| | diphosphate synthase (FPS2) |
| | BC010004, Homo sapiens, farnesyl diphosphate |
| | synthase (farnesyl pyrophosphate synthetase, |
| | dimethylallyltranstransferase, |
| | geranyltranstransferase), clone MGC 15352 IMAGE, |
| | 4132071, mRNA, complete cds |
| | AF234168, Dictyostelium discoideum farnesyl |
| | diphosphate synthase (Dfps) |
| | L46349, Arabidopsis thaliana farnesyl diphosphate |
| | synthase (FPS2) mRNA, complete cds |
| | L46350, Arabidopsis thaliana farnesyl diphosphate |
| | synthase (FPS2) gene, complete cds |
| | L46367, Arabidopsis thaliana farnesyl diphosphate |
| | synthase (FPS1) gene, alternative products, complete |
| | cds |
| | M89945, Rat farnesyl diphosphate synthase gene, |
| | exons 1-8 |
| | NM_002004, Homo sapiens farnesyl diphosphate |
| | synthase (farnesyl pyrophosphate synthetase, |
| | dimethylallyltranstransferase, |
| | geranyltranstransferase) (FDPS), mRNA |
| | U36376 |
| | Artemisia annua farnesyl diphosphate synthase (fps1) |
| | mRNA, complete cds |
| | XM_001352, Homo sapiens farnesyl diphosphate |
| | synthase (farnesyl pyrophosphate synthetase, |
| | dimethylallyltranstransferase, |
| | geranyltranstransferase) (FDPS), mRNA |
| | XM_034497 |
| | Homo sapiens farnesyl diphosphate synthase |
| | (farnesyl pyrophosphate synthetase, |
| | dimethylallyltranstransferase, |
| | geranyltranstransferase) (FDPS), mRNA |
| | XM_034498 |
| | Homo sapiens farnesyl diphosphate synthase |
| | (farnesyl pyrophosphate synthetase, |
| | dimethylallyltranstransferase, |
| | geranyltranstransferase) (FDPS), mRNA |
| | XM_034499 |
| | Homo sapiens farnesyl diphosphate synthase |
| | (farnesyl pyrophosphate synthetase, |
| | dimethylallyltranstransferase, |
| | geranyltranstransferase) (FDPS), mRNA |
| | XM_034500 |
| | Homo sapiens farnesyl diphosphate synthase |
| | (farnesyl pyrophosphate synthetase, |
| | dimethylallyltranstransferase, |
| | geranyltranstransferase) (FDPS), mRNA |
| crtN | X73889, S. aureus |
| crtE (GGPP | AB000835, Arabidopsis thaliana |
| Synthase) | AB016043 and AB019036, Homo sapiens |
| | AB016044, Mus musculus |
| | AB027705 and AB027706, Daucus carota |
| | AB034249, Croton sublyratus |
| | AB034250, Scoparia dulcis |
| | AF020041, Helianthus annuus |
| | AF049658, Drosophila melanogaster signal |
| | recognition particle 19 kDa protein (srp19) gene, |
| | partial sequence; and geranylgeranyl pyrophosphate |
| | synthase (quemao) gene, complete cds |
| | AF049659, Drosophila melanogaster geranylgeranyl |
| | pyrophosphate synthase mRNA, complete cds |
| | AF139916, Brevibacterium linens |
| | AF279807, Penicillium paxilli geranylgeranyl |
| | pyrophosphate synthase (ggs1) gene, complete |
| | AF279808 |
| | Penicillium paxilli dimethylallyl tryptophan synthase |
| | (paxD) gene, partial cds; and cytochrome P450 |
| | monooxygenase (paxQ), cytochrome P450 |
| | monooxygenase (paxP), PaxC (paxC), |
| | monooxygenase (paxM), geranylgeranyl |
| | pyrophosphate synthase (paxG), PaxU (paxU), and |
| | metabolite transporter (paxT) genes, complete cds |
| | AJ010302, Rhodobacter sphaeroides |
| | AJ133724, Mycobacterium aurum |
| | AJ276129, Mucor circinelloides f. lusitanicus carG |
| | gene for geranylgeranyl pyrophosphate synthase, |
| | exons 1-6 |
| | D85029 |
| | Arabidopsis thaliana mRNA for geranylgeranyl |
| | pyrophosphate synthase, partial cds |
| | L25813, Arabidopsis thaliana |
| | L37405, Streptomyces griseus geranylgeranyl |
| | pyrophosphate synthase (crtB), phytoene desaturase |
| | (cftE) and phytoene synthase (cftl) genes, complete |
| | cds |
| | U15778, Lupinus albus geranylgeranyl |
| | pyrophosphate synthase (ggps1) mRNA, complete |
| | cds |
| | U44876, Arabidopsis thaliana pregeranylgeranyl |
| | pyrophosphate synthase (GGPS2) mRNA, complete |
| | cds |
| | X92893, C. roseus |
| | X95596, S. griseus |
| | X98795, S. alba |
| | Y15112, Paracoccus marcusii |
| crtX | D90087, E. uredovora |
| | M87280 and M90698, Pantoea agglomerans |
| crtY | AF139916, Brevibacterium linens |
| | AF152246, Citrus x paradisi |
| | AF218415, Bradyrhizobium sp. ORS278 |
| | AF272737, Streptomyces griseus strain IFO13350 |
| | AJ133724, Mycobacterium aurum |
| | AJ250827, Rhizomucor circinelloides f. lusitanicus |
| | carRP gene for lycopene cyclase/phytoene synthase, |
| | exons 1-2 |
| | AJ276965, Phycomyces blakesleeanus carRA gene |
| | for phytoene synthase/lycopene cyclase, exons 1-2 |
| | D58420, Agrobacterium aurantiacum |
| | D83513, Erythrobacter longus |
| | L40176, Arabidopsis thaliana lycopene cyclase |
| | (LYC) mRNA, complete cds |
| | M87280, Pantoea agglomerans |
| | U50738, Arabodopsis thaliana lycopene epsilon |
| | cyclase mRNA, complete cds |
| | U50739 |
| | Arabidosis thaliana lycopene β cyclase mRNA, |
| | complete cds |
| | U62808, Flavobacterium ATCC21588 |
| | X74599 |
| | Synechococcus sp. lcy gene for lycopene cyclase |
| | X81787 |
| | N. tabacum CrtL-1 gene encoding lycopene cyclase |
| | X86221, C. annuum |
| | X86452, L. esculentum mRNA for lycopene β-cyclase |
| | X95596, S. griseus |
| | X98796, N. pseudonarcissus |
| crtl | AB046992, Citrus unshiu CitPDS1 mRNA for |
| | phytoene desaturase, complete cds |
| | AF039585 |
| | Zea mays phytoene desaturase (pds1) gene promoter |
| | region and exon 1 |
| | AF049356 |
| | Oryza sativa phytoene desaturase precursor (Pds) |
| | mRNA, complete cds |
| | AF139916, Brevibacterium linens |
| | AF218415, Bradyrhizobium sp. ORS278 |
| | AF251014, Tagetes erecta |
| | AF364515, Citrus x paradisi |
| | D58420, Agrobacterium aurantiacum |
| | D83514, Erythrobacter longus |
| | L16237, Arabidopsis thaliana |
| | L37405, Streptomyces griseus geranylgeranyl |
| | pyrophosphate synthase (crtB), phytoene desaturase |
| | (cftE) and phytoene synthase (cftI) genes, complete |
| | cds |
| | L39266, Zea mays phytoene desaturase (Pds) |
| | mRNA, complete cds |
| | M64704, Soybean phytoene desaturase |
| | M88683, Lycopersicon esculentum phytoene |
| | desaturase (pds) mRNA, complete cds |
| | S71770, carotenoid gene cluster |
| | U37285, Zea mays |
| | U46919, Solanum lycopersicum phytoene desaturase |
| | (Pds) gene, partial cds |
| | U62808, Flavobacterium ATCC21588 |
| | X55289, Synechococcus pds gene for phytoene |
| | desaturase |
| | X59948, L. esculentum |
| | X62574, Synechocystis sp. pds gene for phytoene |
| | desaturase |
| | X68058 |
| | C. annuum pds1 mRNA for phytoene desaturase |
| | X71023 |
| | Lycopersicon esculentum pds gene for phytoene |
| | desaturase |
| | X78271, L. esculentum (Ailsa Craig) PDS gene |
| | X78434, P. blakesleeanus (NRRL1555) carB gene |
| | X78815, N. pseudonarcissus |
| | X86783, H. pluvialis |
| | Y14807, Dunaliella bardawil |
| | Y15007, Xanthophyllomyces dendrorhous |
| | Y15112, Paracoccus marcusii |
| | Y15114, Anabaena PCC7210 crtP gene |
| | Z11165, R. capsulatus |
| crtB | AB001284, Spirulina platensis |
| | AB032797, Daucus carota PSY mRNA for phytoene |
| | synthase, complete cds |
| | AB034704, Rubrivivax gelatinosus |
| | AB037975, Citrus unshiu |
| | AF009954, Arabidopsis thaliana phytoene synthase |
| | (PSY) gene, complete cds |
| | AF139916, Brevibacterium linens |
| | AF152892, Citrus x paradisi |
| | AF218415, Bradyrhizobium sp. ORS278 |
| | AF220218, Citrus unshiu phytoene synthase (Psy1) |
| | mRNA, complete cds |
| | AJ010302, Rhodobacter |
| | AJ133724, Mycobacterium aurum |
| | AJ278287, Phycomyces blakesleeanus carRA gene |
| | for lycopene cyclase/phytoene synthase, |
| | AJ304825 |
| | Helianthus annuus mRNA for phytoene synthase (psy |
| | gene) |
| | AJ308385 |
| | Helianthus annuus mRNA for phytoene synthase (psy |
| | gene) |
| | D58420, Agrobacterium aurantiacum |
| | L23424 |
| | Lycopersicon esculentum phytoene synthase (PSY2) |
| | mRNA, complete cds |
| | L25812, Arabidopsis |
| | L37405, Streptomyces griseus geranylgeranyl |
| | pyrophosphate synthase (crtB), phytoene desaturase |
| | (cftE) and phytoene synthase (cftI) genes, complete |
| | cds |
| | M38424 |
| | Pantoea agglomerans phytoene synthase (crtE) |
| | gene, complete cds |
| | M87280, Pantoea agglomerans |
| | S71770, carotenoid gene cluster |
| | U32636 |
| | Zea mays phytoene synthase (Y1) gene, complete |
| | cds |
| | U62808, Flavobacterium ATCC21588 |
| | U87626, Rubrivivax gelatinosus |
| | U91900, Dunaliella bardawil |
| | X52291, Rhodobacter capsulatus |
| | X60441, L. esculentum GTom5 gene for phytoene |
| | synthase |
| | X63873 |
| | Synechococcus PCC7942 pys gene for phytoene |
| | synthase |
| | X68017 |
| | C. annuum psy1 mRNA for phytoene synthase |
| | X69172 |
| | Synechocystis sp. pys gene for phytoene synthase |
| | X78814, N. pseudonarcissus |
| crtZ | D58420, Agrobacterium aurantiacum |
| | D58422, Alcaligenes sp. |
| | D90087, E. uredovora |
| | M87280, Pantoea agglomerans |
| | U62808, Flavobacterium ATCC21588 |
| | Y15112, Paracoccus marcusii |
| crtW | AF218415, Bradyrhizobium sp. ORS278 |
| | D45881, Haematococcus pluvialis |
| | D58420, Agrobacterium aurantiacum |
| | D58422, Alcaligenes sp. |
| | X86782, H. pluvialis |
| | Y15112, Paracoccus marcusii |
| crtO | X86782, H. pluvialis |
| | Y15112, Paracoccus marcusii |
| crtU | AF047490, Zea mays |
| | AF121947, Arabidopsis thaliana |
| | AF139916, Brevibacterium linens |
| | AF195507, Lycopersicon esculentum |
| | AF272737, Streptomyces griseus strain IFO13350 |
| | AF372617, Citrus x paradisi |
| | AJ133724, Mycobacterium aurum |
| | AJ224683, Narcissus pseudonarcissus |
| | D26095 and U38550, Anabaena sp. |
| | X89897, C. annuum |
| | Y15115, Anabaena PCC7210 crtQ gene |
| crtA | AJ010302, Rhodobacter sphaeroides |
| (spheroidene | Z11165 and X52291, Rhodobacter capsulatus |
| monooxygenase) | |
| crtC | AB034704, Rubrivivax gelatinosus |
| | AF195122 and AJ010302, Rhodobacter sphaeroides |
| | AF287480, Chlorobium tepidum |
| | U73944, Rubrivivax gelatinosus |
| | X52291 and Z11165, Rhodobacter capsulatus |
| | Z21955, M. xanthus |
| crtD | AJ010302 and X63204, Rhodobacter sphaeroides |
| (carotenoid 3,4- | U73944, Rubrivivax gelatinosus |
| desaturase | X52291 and Z11165, Rhodobacter capsulatus |
| crtF | AB034704, Rubrivivax gelatinosus |
| (1-OH-carotenoid | AF288602, Chloroflexus aurantiacus |
| methylase) | AJ010302, Rhodobacter sphaeroides |
| | X52291 and Z11165, Rhodobacter capsulatus |

The most preferred source of genes for the lower carotenoid biosynthetic pathway in the present invention are from a variety of sources. The “ispA” gene (SEQ ID NO:19) is native to Methylomonas 16a, as the organism produces respiratory quinones and a 30-carbon carotenoid via the 2-C-methyl-D-erythritol 4-phosphate (MEP) pathway. However, Methylomonas does not synthesize the desired 40-carbon carotenoids. FPP is the end-product of the MEP pathway in Methylomonas 16A and is subsequently converted to its natural 30-carbon carotenoid by the action of the sqs, crtN1 and crtN2 gene products. As a native gene to the preferred host organism, the ispA gene (SEQ ID NO:19) is the most preferred source of the gene for the present invention.

The majority of the most preferred source of crt genes are primarily from Panteoa stewartii. Sequences of these preferred genes are presented as the following SEQ ID numbers: the crtE gene (SEQ ID NO:25), the crtX gene (SEQ ID NO:27), crtY (SEQ ID NO:29), the crtI gene (SEQ ID NO:31), the crtB gene (SEQ ID NO:33) and the crtZ gene (SEQ ID NO:35). Additionally, the crtO gene isolated from Rhodococcus erythropolis AN12 and presented as SEQ ID NO:37 is preferred in combination with other genes for the present invention.

By using various combinations of the genes presented in Table 3 and the preferred genes of the present invention, innumerable different carotenoids and carotenoid derivatives could be made using the methods of the present invention, provided sufficient sources of IPP are available in the host organism. For example, the gene cluster crtEXYIB enables the production of β-carotene. Addition of the crt Z to crtEXYIB enables the production of zeaxanthin, while the crt EXYIBZO cluster leads to production of astaxanthin and canthaxanthin.

It is envisioned that useful products of the present invention will include any carotenoid compound as defined herein including but not limited to antheraxanthin, adonixanthin, astaxanthin, canthaxanthin, capsorubrin, β-cryptoxanthin alpha-carotene, beta-carotene, epsilon-carotene, echinenone, gamma-carotene, zeta-carotene, alpha-cryptoxanthin, diatoxanthin, 7,8-didehydroastaxanthin, fucoxanthin, fucoxanthinol, isorenieratene, lactucaxanthin, lutein, lycopene, neoxanthin, neurosporene, hydroxyneurosporene, peridinin, phytoene, rhodopin, rhodopin glucoside, siphonaxanthin, spheroidene, spheroidenone, spirilloxanthin, uriolide, uriolide acetate, violaxanthin, zeaxanthin-β-diglucoside, and zeaxanthin. Additionally the invention encompasses derivitization of these molecules to create hydroxy-, methoxy-, oxo-, epoxy-, carboxy-, or aldehydic functional groups, or glycoside esters, or sulfates.

Construction of Recombinant C1 Metabolizing Microorganisms

Methods for introduction of genes encoding the appropriate upper isoprene pathway genes or lower carotenoid biosynthetic pathway genes into a suitable C1 metabolizing host are common. Microbial expression systems and expression vectors containing regulatory sequences suitable for expression of heterologus genes in C1 metabolizing hosts are known. Any of these could be used to construct chimeric genes for expression of any of the above mentioned carotenoid biosynthetic genes. These chimeric genes could then be introduced into appropriate hosts via transformation to provide high level expression of the enzymes.

Vectors or cassettes useful for the transformation of suitable host cells are available. For example several classes of promoters may be used for the expression of genes encoding the present carotenoid biosynthetic genes in C1 metabolizers including, but not limited to endogenous promoters such as the deoxy-xylulose phosphate synthase or methanol dehydrogenase operon promoter (Springer et al. (1998) FEMS Microbiol Lett 160:119-124), the promoter for polyhydroxyalkanoic acid synthesis (Foellner et al. Appl. Microbiol. Biotechnol. (1993) 40:284-291), or promoters identified from native plasmids in methylotrophs (EP 296484). In addition to these native promoters, non-native promoters may also be used, as for example the promoter for the lactose operon Plac (Toyama et al. Microbiology (1997) 143:595-602; EP 62971) or a hybrid promoter such as Ptrc (Brosius et al. (1984) Gene 27:161-172). Similarly, promoters associated with antibiotic resistance, e.g. kanamycin (Springer et al. (1998) FEMS Microbiol Lett 160:119-124; Ueda et al. Appl. Environ. Microbiol. (1991) 57:924-926) or tetracycline (U.S. Pat. No. 4,824,786), are also suitable.

Once the specific regulatory element is selected, the promoter-gene cassette can be introduced into a C1 metabolizer on a plasmid containing either a replicon for episomal expression (Brenner et al. Antonie Van Leeuwenhoek (1991) 60:4348; Ueda et al. Appl. Environ. Microbiol. (1991) 57:924-926) or homologous regions for chromosomal integration (Naumov et al. Mol. Genet. Mikrobiol. Virusol. (1986) 11:44-48).

Typically, the vector or cassette contains sequences directing transcription and translation of the relevant gene, a selectable marker, and sequences allowing autonomous replication or chromosomal integration. Suitable vectors comprise a region 5′ of the gene which harbors transcriptional initiation controls and a region 3′ of the DNA fragment which controls transcriptional termination. It is most preferred when both control regions are derived from genes homologous to the transformed host cell, although it is to be understood that such control regions need not be derived from the genes native to the specific species chosen as a production host.

Where accumulation of a specific carotenoid is desired it may be necessary to reduce or eliminate the expression of certain genes in the target pathway or in competing pathways that may serve as competing sinks for energy or carbon. Alternatively, it may be useful to over-express various genes upstream of desired carotenoid intermediates to enhance production.

Methods of up-regulating and down-regulating genes for this purpose have been explored. Where sequence of the gene to be disrupted is known, one of the most effective methods gene down regulation is targeted gene disruption where foreign DNA is inserted into a structural gene so as to disrupt transcription. This can be effected by the creation of genetic cassettes comprising the DNA to be inserted (often a genetic marker) flanked by sequence having a high degree of homology to a portion of the gene to be disrupted. Introduction of the cassette into the host cell results in insertion of the foreign DNA into the structural gene via the native DNA replication mechanisms of the cell. (See for example Hamilton et al. (1989) J. Bacteriol. 171:4617-4622, Balbas et al. (1993) Gene 136:211-213, Gueldener et al. (1996) Nucleic Acids Res. 24:2519-2524, and Smith et al. (1996) Methods Mol. Cell. Biol. 5:270-277.)

Antisense technology is another method of down regulating genes where the sequence of the target gene is known. To accomplish this, a nucleic acid segment from the desired gene is cloned and operably linked to a promoter such that the anti-sense strand of RNA will be transcribed. This construct is then introduced into the host cell and the antisense strand of RNA is produced. Antisense RNA inhibits gene expression by preventing the accumulation of mRNA which encodes the protein of interest. The person skilled in the art will know that special considerations are associated with the use of antisense technologies in order to reduce expression of particular genes. For example, the proper level of expression of antisense genes may require the use of different chimeric genes utilizing different regulatory elements known to the skilled artisan.

Although targeted gene disruption and antisense technology offer effective means of down regulating genes where the sequence is known, other less specific methodologies have been developed that are not sequence based. For example, cells may be exposed to a UV radiation and then screened for the desired phenotype. Mutagenesis with chemical agents is also effective for generating mutants and commonly used substances include chemicals that affect non-replicating DNA such as HNO₂ and NH₂OH, as well as agents that affect replicating DNA such as acridine dyes, notable for causing frameshift mutations. Specific methods for creating mutants using radiation or chemical agents are well documented in the art. See for example Thomas D. Brock in Biotechnology: A Textbook of Industrial Microbiology, Second Edition (1989) Sinauer Associates, Inc., Sunderland, Mass., or Deshpande, Mukund V., Appl. Biochem. Biotechnol., 36, 227, (1992).

Another non-specific method of gene disruption is the use of transposoable elements or transposons. Transposons are genetic elements that insert randomly in DNA but can be latter retrieved on the basis of sequence to determine where the insertion has occurred. Both in vivo and in vitro transposition methods are known. Both methods involve the use of a transposable element in combination with a transposase enzyme. When the transposable element or transposon, is contacted with a nucleic acid fragment in the presence of the transposase, the transposable element will randomly insert into the nucleic acid fragment. The technique is useful for random mutagenesis and for gene isolation, since the disrupted gene may be identified on the basis of the sequence of the transposable element. Kits for in vitro transposition are commercially available (see for example The Primer Island Transposition Kit, available from Perkin Elmer Applied Biosystems, Branchburg, N.J., based upon the yeast Ty1 element; The Genome Priming System, available from New England Biolabs, Beverly, Mass.; based upon the bacterial transposon Tn7; and the EZ::TN Transposon Insertion Systems, available from Epicentre Technologies, Madison, Wis., based upon the Tn5 bacterial transposable element.

In the context of the present invention the disruption of certain genes in the terpenoid pathway may enhance the accumulation of specific carotenoids however, the decision of which genes to disrupt would need to be determined on an empirical basis. Candidate genes may include one or more of the prenyltransferase genes which, as described earlier, which catalyze the successive condensation of isopentenyl diphosphate resulting in the formation of prenyl diphosphates of various chain lengths (multiples of C-5 isoprene units). Other candidate genes for disruption would include any of those which encode proteins acting upon the terpenoid backbone prenyl diphosphates.

Similarly, over-expression of certain genes upstream of the desired product will be expected to have the effect of increasing the production of that product. For example, may of the genes in the upper isoprenoid pathway (D-1-deoxyxylulose-5-phosphate synthase (Dxs), D-1-deoxyxylulose-5-phosphate reductoisomerase (Dxr), 2C-methyl-d-erythritol cytidylyltransferase (IspD), 4-diphosphocytidyl-2-C-methylerythritol kinase (IspE), 2C-methyl-d-erythritol 2,4-cyclodiphosphate synthase (IspF), CTP synthase (PyrG) and lytB) could be expressed on multicopy plasmids, or under the influence of strong non-native promoters. In this fashion the levels of desired carotenoids may be enhanced.

Industrial Production of Carotenoids

Where commercial production of carotenoid compounds is desired according to the present invention, a variety of culture methodologies may be applied. For example, large-scale production of a specific gene product, over-expressed from a recombinant microbial host may be produced by both batch or continuous culture methodologies.

A classical batch culturing method is a closed system where the composition of the media is set at the beginning of the culture and not subject to artificial alterations during the culturing process. Thus, at the beginning of the culturing process the media is inoculated with the desired organism or organisms and growth or metabolic activity is permitted to occur adding nothing to the system. Typically, however, a “batch” culture is batch with respect to the addition of carbon source and attempts are often made at controlling factors such as pH and oxygen concentration. In batch systems the metabolite and biomass compositions of the system change constantly up to the time the culture is terminated. Within batch cultures cells moderate through a static lag phase to a high growth log phase and finally to a stationary phase where growth rate is diminished or halted. If untreated, cells in the stationary phase will eventually die. Cells in log phase are often responsible for the bulk of production of end product or intermediate in some systems. Stationary or post-exponential phase production can be obtained in other systems.

A variation on the standard batch system is the Fed-Batch system. Fed-Batch culture processes are also suitable in the present invention and comprise a typical batch system with the exception that the substrate is added in increments as the culture progresses. Fed-Batch systems are useful when catabolite repression is apt to inhibit the metabolism of the cells and where it is desirable to have limited amounts of substrate in the media. Measurement of the actual substrate concentration in Fed-Batch systems is difficult and is therefore estimated on the basis of the changes of measurable factors such as pH, dissolved oxygen and the partial pressure of waste gases such as CO₂. Batch and Fed-Batch culturing methods are common and well known in the art and examples may be found in Thomas D. Brock in Biotechnology: A Textbook of Industrial Microbiology, Second Edition (1989) Sinauer Associates, Inc., Sunderland, Mass., or Deshpande, Mukund V., Appl. Biochem. Biotechnol., 36, 227, (1992), herein incorporated by reference.

Commercial production of carotenoids using C1 metabolizers may also be accomplished with a continuous culture. A continuous culture is an open system where a defined culture media is added continuously to a bioreactor and an equal amount of conditioned media is removed simultaneously for processing. Continuous cultures generally maintain the cells at a constant high liquid phase density where cells are primarily in log phase growth. Alternatively continuous culture may be practiced with immobilized cells where carbon and nutrients are continuously added, and valuable products, by-products or waste products are continuously removed from the cell mass. Cell immobilization may be performed using a wide range of solid supports composed of natural and/or synthetic materials.

Continuous or semi-continuous culture allows for the modulation of one factor or any number of factors that affect cell growth or end product concentration. For example, one method will maintain a limiting nutrient such as the carbon source or nitrogen level at a fixed rate and allow all other parameters to moderate. In other systems a number of factors affecting growth can be altered continuously while the cell concentration, measured by media turbidity, is kept constant. Continuous systems strive to maintain steady state growth conditions and thus the cell loss due to media being drawn off must be balanced against the cell growth rate in the culture. Methods of modulating nutrients and growth factors for continuous culture processes as well as techniques for maximizing the rate of product formation are well known in the art of industrial microbiology and a variety of methods are detailed by Brock, supra.

Fermentation media in the present invention must contain suitable carbon substrates for C1 metabolizing organisms. Suitable substrates may include but are not limited to one-carbon substrates such as carbon dioxide, methane or methanol for which metabolic conversion into key biochemical intermediates has been demonstrated. In addition to one and two carbon substrates, methylotrophic organisms are also known to utilize a number of other carbon containing compounds such as methylamine, glucosamine and a variety of amino acids for metabolic activity. For example, methylotrophic yeast are known to utilize the carbon from methylamine to form trehalose or glycerol (Bellion et al., Microb. Growth C1 Compd., [Int. Symp.], 7th (1993), 415-32. Editor(s): Murrell, J. Collin; Kelly, Don P. Publisher: Intercept, Andover, UK). Similarly, various species of Candida will metabolize alanine or oleic acid (Sulter et al., Arch. Microbiol. 153:485-489 (1990)). Hence it is contemplated that the source of carbon utilized in the present invention may encompass a wide variety of carbon containing substrates and will only be limited by the choice of organism.

### EXAMPLES

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions.

General Methods

Standard recombinant DNA and molecular cloning techniques used in the Examples are well known in the art and are described by Sambrook, J., Fritsch, E. F. and Maniatis, T. Molecular Cloning: A Laboratory Manual; Cold Spring Harbor Laboratory Press: Cold Spring Harbor, (1989) (Maniatis) and by T. J. Silhavy, M. L. Bennan, and L. W. Enquist, Experiments with Gene Fusions, Cold Spring Harbor Laboratory, Cold Spring Harbor, N.Y. (1984) and by Ausubel, F. M. et al., Current Protocols in Molecular Biology, pub. by Greene Publishing Assoc. and Wiley-Interscience (1987).

Materials and methods suitable for the maintenance and growth of bacterial cultures are well known in the art. Techniques suitable for use in the following examples may be found as set out in Manual of Methods for General Bacteriology (Phillipp Gerhardt, R. G. E. Murray, Ralph N. Costilow, Eugene W. Nester, Willis A. Wood, Noel R. Krieg and G. Briggs Phillips, eds), American Society for Microbiology, Washington, D.C. (1994)) or by Thomas D. Brock in Biotechnology: A Textbook of Industrial Microbiology, Second Edition, Sinauer Associates, Inc., Sunderland, Mass. (1989). All reagents, restriction enzymes and materials used for the growth and maintenance of bacterial cells were obtained from Aldrich Chemicals (Milwaukee, Wis.), DIFCO Laboratories (Detroit, Mich.), GIBCO/BRL (Gaithersburg, Md.), or Sigma Chemical Company (St. Louis, Mo.) unless otherwise specified.

Manipulations of genetic sequences were accomplished using the suite of programs available from the Genetics Computer Group Inc. (Wisconsin Package Version 9.0, Genetics Computer Group (GCG), Madison, Wis.). Where the GCG program “Pileup” was used the gap creation default value of 12, and the gap extension default value of 4 were used. Where the CGC “Gap” or “Bestfit” programs were used the default gap creation penalty of 50 and the default gap extension penalty of 3 were used. In any case where GCG program parameters were not prompted for, in these or any other GCG program, default values were used.

The meaning of abbreviations is as follows: “h” means hour(s), “min” means minute(s), “sec” means second(s), “d” means day(s), “mL” means milliliters, “L” means liters.

Microbial Cultivation, Preparation of Cell Suspensions, and Associated Analyses for Methylomonas 16a

The following conditions were used throughout the experimental Examples for treatment of Methylomonas 16a, unless conditions were specifically specified otherwise.

Methylomonas 16a is typically grown in serum stoppered Wheaton bottles (Wheaton Scientific, Wheaton Ill.) using a gas/liquid ratio of at least 8:1 (i.e. 20 mL of Nitrate liquid “BTZ-3” media of 160 mL total volume). The standard gas phase for cultivation contained 25% methane in air. These conditions comprise growth conditions and the cells are referred to as growing cells. In all cases, the cultures were grown at 30° C. with constant shaking in a Lab-Line rotary shaker unless otherwise specified.

Nitrate Medium for Methylomonas 16A

Nitrate liquid medium, also referred to herein as “defined medium” or “BTZ-3” medium was comprised of various salts mixed with Solution 1 as indicated below (Tables 4 and 5) or where specified the nitrate was replaced with 15 mM ammonium chloride. Solution 1 provides the comosition for 100 fold concentrated stock solution of trace minerals.

TABLE 4
Solution 1*

| | | Conc. | |
|---|---|---|---|
| | MW | (mM) | g per L |
| Nitriloacetic acid | 191.1 | 66.9 | 12.8 |
| CuCl₂ × 2H₂O | 170.48 | 0.15 | 0.0254 |
| FeCl₂ × 4H₂O | 198.81 | 1.5 | 0.3 |
| MnCl₂ × 4H₂O | 197.91 | 0.5 | 0.1 |
| CoCl₂ × 6H₂O | 237.9 | 1.31 | 0.312 |
| ZnCl₂ | 136.29 | 0.73 | 0.1 |
| H₃BO₃ | 61.83 | 0.16 | 0.01 |
| Na₂MoO₄ × 2H₂O | 241.95 | 0.04 | 0.01 |
| NiCl₂ × 6H₂O | 237.7 | 0.77 | 0.184 |

*Mix the gram amounts designated above in 900 mL of H₂O, adjust to pH = 7, and add H₂O to an end volume of 1 L. Keep refrigerated.

TABLE 5
Nitrate liquid medium (BTZ-3)**

| | | Conc. | |
|---|---|---|---|
| | MW | (mM) | g per L |
| NaNO₃ | 84.99 | 10 | 0.85 |
| KH₂PO₄ | 136.09 | 3.67 | 0.5 |
| Na₂SO₄ | 142.04 | 3.52 | 0.5 |
| MgCl₂ × 6H₂O | 203.3 | 0.98 | 0.2 |
| CaCl₂ × 2H₂O | 147.02 | 0.68 | 0.1 |
| 1 M HEPES (pH 7) | 238.3 | | 50 mL |
| Solution 1 | | | 10 mL |

**Dissolve in 900 mL H₂O. Adjust to pH = 7, and add H₂O to give 1 L. For agar plates: Add 15 g of agarose in 1 L of medium, autoclave, let cool down to 50° C., mix, and pour plates.

Assessment of Microbial Growth and Conditions for Harvesting Cells

Cells obtained for experimental purposes were allowed to grow to maximum optical density (O.D. 660˜1.0). Harvested cells were obtained by centrifugation in a Sorval RC-5B centrifuge using a SS-34 rotor at 6000 rpm for 20 min. These cell pellets were resuspended in 50 mM HEPES buffer pH 7. These cell suspensions are referred to as washed, resting cells.

Microbial growth was assessed by measuring the optical density of the culture at 660 nm in an Ultrospec 2000 UV/Vis is spectrophotometer (Pharmacia Biotech, Cambridge England) using a 1 cm light path cuvet. Alternatively microbial growth was assessed by harvesting cells from the culture medium by centrifugation as described above and, resuspending the cells in distilled water with a second centrifugation to remove medium salts. The washed cells were then dried at 105° C. overnight in a drying oven for dry weight determination.

Methane concentration was determined as described by Emptage et al. (1997 Env. Sci. Technol. 31:732-734), hereby incorporated by reference.

Nitrate and Nitrite Assays

1 mL samples of cell culture were taken and filtered through a 0.2 micron Acrodisc filter to remove cells. The filtrate from this step contains the nitrite or nitrate to be analyzed. The analysis was performed on a Dionex ion chromatograph 500 system (Dionex, Sunnyvale Calif.) with an AS3500 autosampler. The column used was a 4 mm Ion-Pac AS11-HC separation column with an AG-AC guard column and an ATC trap column. All columns are provided by Dionex.

The mobile phase was a potassium hydroxide gradient from 0 to 50 mM potassium hydroxide over a 12 min time interval. Cell temperature was 35° C. with a flow rate of 1 mL/min.

HPLC Analysis of Carotenoid Content

Cell pellets were extracted with 1 ml acetone by vortexing for 1 min and intermittent vortexing over the next 30 min. Cell debris was removed by centrifugation at 14,000×g for 10 min and the supernatants was collected and passed through a 0.45 μM filter. A Beckman System Gold® HPLC with Beckman Gold Nouveau Software (Columbia, Md.) was used for the study. The crude extraction (0.1 mL) was loaded onto a 125×4 mm RP8 (5 μm particles) column with corresponding guard column (Hewlett-Packard, San Fernando, Calif.). The flow rate was 1 mL/min, while the solvent program used was: 0-11.5 min 40% water/60% methanol; 11.5-20 min 100% methanol; 20-30 min 40% water/60% methanol. The spectral data was collected by a Beckman photodiode array detector (model 168).

### Example 1

### Isolation and Sequencing Of Methylomonas 16a

The original environmental sample containing the isolate was obtained from pond sediment. The pond sediment was inoculated directly into defined medium with ammonium as nitrogen source under 25% methane in air. Methane was the sole source of carbon and energy. Growth was followed until the optical density at 660 nm was stable, whereupon the culture was transferred to fresh medium such that a 1:100 dilution was achieved. After 3 successive transfers with methane as sole carbon and energy source, the culture was plated onto growth agar with ammonium as nitrogen source and incubated under 25% methane in air. Many methanotrophic bacterial species were isolated in this manner. However, Methylomonas 16a was selected as the organism to study due to its rapid growth of colonies, large colony size, ability to grow on minimal media, and pink pigmentation indicative of an active biosynthetic pathway for carotenoids.

Genomic DNA was isolated from Methylomonas 16a according to standard protocols. Genomic DNA and library construction were prepared according to published protocols (Fraser et al., The Minimal Gene Complement of Mycoplasma genitalium; Science 270 (5235):397-403 (1995)). A cell pellet was resuspended in a solution containing 100 mM Na-EDTA pH 8.0, 10 mM Tris-HCl pH 8.0, 400 mM NaCl, and 50 mM MgCl₂.

Genomic DNA preparation After resuspension, the cells were gently lysed in 10% SDS, and incubated for 30 min at 55° C. After incubation at room temperature, proteinase K was added to 100 μg/mL and incubated at 37° C. until the suspension was clear. DNA was extracted twice with Tris-equilibrated phenol and twice with chloroform. DNA was precipitated in 70% ethanol and resuspended in a solution containing 10 mM Tris-HCl and 1 mM Na-EDTA (TE), pH 7.5. The DNA solution was treated with a mix of RNAases, then extracted twice with Tris-equilibrated phenol and twice with chloroform. This was followed by precipitation in ethanol and resuspension in TE.

Library construction 200 to 500 μg of chromosomal DNA was resuspended in a solution of 300 mM sodium acetate, 10 mM Tris-HCl, 1 mM Na-EDTA, and 30% glycerol, and sheared at 12 psi for 60 sec in an Aeromist Downdraft Nebulizer chamber (IBI Medical products, Chicago, Ill.). The DNA was precipitated, resuspended and treated with Bal31 nuclease. After size fractionation, a fraction (2.0 kb, or 5.0 kb) was excised and cleaned, and a two-step ligation procedure was used to produce a high titer library with greater than 99% single inserts.

Sequencing A shotgun sequencing strategy approach was adopted for the sequencing of the whole microbial genome (Fleischmann, R. et al., Whole-Genome Random sequencing and assembly of Haemophilus influenzae Rd Science 269(5223):496-512 (1995)). Sequence was generated on an ABI Automatic sequencer using dye terminator technology (U.S. Pat. No. 5,366,860; EP 272,007) using a combination of vector and insert-specific primers. Sequence editing was performed in either DNAStar (DNA Star Inc.) or the Wisconsin GCG program (Wisconsin Package Version 9.0, Genetics Computer Group (GCG), Madison, Wis.) and the CONSED package (version 7.0). All sequences represent coverage at least two times in both directions.

### Example 2

### Identification and Characterization of Bacterial Genes from Methylomonas

All sequences from Example 1 were identified by conducting BLAST (Basic Local Alignment Search Tool; Altschul, S. F., et al., (1993) J. Mol. Biol. 215:403-410; see also www.ncbi.nlm.nih.gov/BLAST/) searches for similarity to sequences contained in the BLAST “nr” database (comprising all non-redundant GenBank CDS translations, sequences derived from the 3-dimensional structure Brookhaven Protein Data Bank, the SWISS-PROT protein sequence database, EMBL, and DDBJ databases). The sequences were analyzed for similarity to all publicly available DNA sequences contained in the “nr” database using the BLASTN algorithm provided by the National Center for Biotechnology Information (NCBI). The DNA sequences were translated in all reading frames and compared for similarity to all publicly available protein sequences contained in the “nr” database using the BLASTX algorithm (Gish, W. and States, D. J. (1993) Nature Genetics 3:266-272) provided by the NCBI. All comparisons were done using either the BLASTNnr or BLASTXnr algorithm.

The results of these BLAST comparisons are given below in Table 6 for many critical genes of the present invention. Table 6 summarizes the sequence to which each Methylomonas gene has the most similarity (presented as % similarities, % identities, and expectation values). The table displays data based on the BLASTXnr algorithm with values reported in expect values. The Expect value estimates the statistical significance of the match, specifying the number of matches, with a given score, that are expected in a search of a database of this size absolutely by chance.

TABLE 6
Identification of Critical Methylomonas Genes Based on Sequence Homology

| Gene | | SEQ | SEQ ID | % | % | | |
|---|---|---|---|---|---|---|---|
| Name | Similarity Identified | ID | peptide | Identity^{a} | Similarity^{b} | E-value^{c} | Citation |
| Phosphofructokinase | Phosphofructokinase | 1 | 2 | 63% | 83% | 1.7e−97 | Ladror et al., J. Biol. Chem. 266, |
| pyrophosphate | pyrophosphate | | | | | | 16550-16555 (1991) |
| dependent | dependent | | | | | | |
| | gi\|150931\|gb\|AAA25675.1\| | | | | | | |
| | (M67447) | | | | | | |
| KHG/KDPG | (AL352972) | 3 | 4 | 59% | 72% | 1e−64 | Redenbach et al., Mol. Microbiol. 21 |
| | KHG/KDPG aldolase | | | | | | (1), 77-96 (1996) |
| | Streptomyces | | | | | | |
| | coelicolor | | | | | | |
| dxs | 1-deoxyxylulose-5- | 5 | 6 | 60% | 86% | 5.7e−149 | Lois et al., Proc. Natl. Acad. Sci. |
| | phosphate synthase | | | | | | USA. 95 (5), 2105-2110 (1998) |
| | (E. coli) | | | | | | |
| dxr | 1-deoxy-d-xylulose | 7 | 8 | 55% | 78% | 3.3e−74 | Takahashi et al., Proc. Natl. Acad. |
| | 5-phosphate | | | | | | USA 95: 9879-9884 (1998) |
| | reductoisomerase | | | | | | |
| | (E. coli) | | | | | | |
| ygbP/ispD | 2C-methyl-d- | 9 | 10 | 52% | 74% | 7.7e−36 | Rohdich et al., Proc Natl Acad Sci |
| | erythritol | | | | | | USA Oct 12, 1999; 96(21): 11758-63 |
| | cytidylyltransferase | | | | | | |
| | (E. coli) | | | | | | |
| ychB/IspE | 4-diphosphocytidyl- | 11 | 12 | 50% | 73% | 8.8e−49 | Luttgen et al., Proc Natl Acad Sci |
| | 2-C-methylerythritol | | | | | | USA. Feb 1, 2000; 97(3): 1062-7. |
| | kinase | | | | | | |
| | (E. coli) | | | | | | |
| ygbB/ispF | 2C-methyl-d- | 13 | 14 | 69% | 84% | 1.6e−36 | Herz et al., Proc Natl Acad Sci USA |
| | erythritol 2,4- | | | | | | Mar 14, 2000; 97(6): 2486-90 |
| | cyclodiphosphate | | | | | | |
| | synthase | | | | | | |
| | (E. coli) | | | | | | |
| pyrG | CTP synthase | 15 | 16 | 67% | 89% | 2.4e−141 | Weng. et al., J. Biol. Chem. |
| | (E. coli) | | | | | | 261: 5568-5574 (1986) |
| lytB | Acinetobacter sp | 17 | 18 | 65 | 87 | 3.4e−75 | Genbank# G.I. 5915671 |
| | BD413 | | | | | | |
| | Putative penicillin | | | | | | |
| | binding protein | | | | | | |
| lspA | Geranyltranstransferase | 19 | 20 | 57% | 78% | 7.8e−56 | Ohto, et al., Plant Mol. Biol. 40 (2), |
| | (also farnesyl- | | | | | | 307-321 (1999) |
| | diphosphate | | | | | | |
| | synthase) | | | | | | |
| | (Synechococcus | | | | | | |
| | elongatus) | | | | | | |
| crtN1 | diapophytoene | 21 | 22 | 34% | 72% | 4e−66 | Xiong, et al., .Proc. Natl. Acad. Sci. |
| | dehydrogenase | | | | | | U.S.A. 95 (25), 14851-14856 (1998) |
| | CrtN-copy 1 | | | | | | |
| | (Heliobacillus | | | | | | |
| | mobilis) | | | | | | |
| crtN2 | Diapophytoene | 23 | 24 | 49% | 78% | 1.3e−76 | Genbank#: X97985 |
| | dehydrogenase | | | | | | |
| | CrtN-copy 2 | | | | | | |
| | (Staphylococcus | | | | | | |
| | aureus) | | | | | | |

### Example 3

### Microarray for Gene Expression in Methylomonas 16a

All bacterial ORFs of Methylomonas were prepared for DNA microarray. The following Example presents the specific protocols utilized for microarray analysis.

Amplification of DNA regions for the construction of DNA microarray. Specific primer pairs were used to amplify each protein specifying ORF of Methylomonas sp. strain 16a. Genomic DNA (10-30 ng) was used as the template. The PCR reactions were performed in the presence of HotStart Taq™ DNA polymerase (Qiagen, Valencia, Calif.) and dNTPs (Gibco BRL Life Science Technologies, Gaithersberg, Md.). Thirty-five cycles of denaturation at 95° C. for 30 sec, annealing at 55° C. for 30 sec, and polymerization at 72° C. for 2 min were conducted. The quality of PCR reactions was checked with electrophresis in a 1% argarose gel. The DNA samples were purified by the high-throughput PCR purification kit from Qiagen.

Arraying amplified ORFs. Before arraying, an equal volume of DMSO (10 μL) and DNA (10 μL) sample was mixed in 384-well microtiter plates. A generation II DNA spotter (Molecular Dynamics, Sunnyvale, Calif.) was used to array the samples onto coated glass slides (Telechem, Sunnyvale, Calif.). Each PCR product was arrayed in duplicate on each slide. After cross-linking by UV light, the slides were stored under vacuum in a desiccator at room temperature.

RNA isolation. Methylomonas 16a was cultured in a defined medium with ammonium or nitrate (10 mM) as a nitrogen source under 25% methane in air. Samples of the minimal medium culture were harvested when the O.D. reached 0.3 at A₆₀₀ (exponential phase). Cell cultures were harvested quickly and ruptured in RLT buffer (Qiagen RNeasy Mini Kit, Valencia, Calif.) with a beads-beater (Bio101, Vista, Calif.). Debris was pelleted by centrifugation for 3 min at 14,000×g at 4° C. RNA isolation was completed using the protocol supplied with this kit. After on-column DNAase treatment, the RNA product was eluted with 50-100 μL RNAase-free water. RNA preparations were stored frozen at either −20 or −80° C.

Synthesis of fluorescent cDNA from total RNA. RNA samples (7 to 15 μg) and random hexamer primers (6 μg; Gibco BRL, Gaithersburg, Md.) were diluted with RNAase-free water to a volume of 25 μL. The sample was denatured at 70° C. for 10 min and then chilled on ice for 30 sec. After adding 14 μL of labeling mixture, the annealing was accomplished by incubation at room temperature for 10 min. The labeling mixture contained 8 μL of 5× enzyme buffer, 4 μL DTT (0.1M), and 2 μL of 20× dye mixture. The dye mixture consisted of 2 mM of each dATP, dGTP, and dTTP, 1 mM dCTP, and 1 mM of Cy3-dCTP or Cy5-dCTP. After adding 1 to 1.5 μL of SuperScript II reverse transcriptase (200 units/mL, Life Technologies Inc., Gaithersberg, Md.), cDNA synthesis was allowed to proceed at 42° C. for 2 hr. The RNA was removed by adding 2 μL NaOH (2.5N) to the reaction. After 10 min of incubation at 37° C., the pH was adjusted with 10 μL of HEPES (2M). The labeled cDNA was then purified with a PCR purification kit (Qiagen, Valencia, Calif.). Labeling efficiency was monitored using either A₅₅₀ for Cy3 incorporation, or A₆₅₀ for Cy5.

Fluorescent labeling of genomic DNA. Genomic DNA was nebulized to approximately 2 kb pair fragments. Genomic DNA (0.5 to 1 μg) was mixed with 6 μg of random hexamers primers (Gibco BRL Life Science Technologies, Gaithersburg, Md.) in 15 μL of water. The mix was denatured by placement in boiling water for 5 min, followed by annealing on ice for 30 sec before transfer to room temperature. Then, 2 μL 5× Buffer 2 (Gibco BRL) and 2 ul dye mixture were added. The components of the dye mixture and the labeling procedure are the same as described above for RNA labeling, except that the Klenow fragment of DNA polymerase I (5 μg/μL, Gibco BRL) was used as the enzyme. After incubation at 37° C. for 2 hr, the labeled DNA probe was purified using a PCR purification kit (Qiagen, Valencia, Calif.).

Hybridization and washing. Slides were first incubated with prehybridization solution containing 3.5×SSC (Gibco BRL, Gaithersberg, Md.), 0.1% SDS (Gibco BRL), 1% bovine serum albumin (BSA, Fraction V, Sigma, St. Louis, Mo.). After prehybridization, hybridization solutions (Molecular Dynamics, Sunnyvale, Calif.) containing labeled probes were added to slides and covered with cover slips. Slides were placed in a humidified chamber in a 42° C. incubator. After overnight hybridization, slides were initially washed for 5 min at room temperature with a washing solution containing 1×SSC, 0.1% SDS and 0.1×SSC, 0.1% SDS. Slides were then washed at 65° C. for 10 min with the same solution for three times. After washing, the slides were dried with a stream of nitrogen gas.

Data Collection and Analysis. The signal generated from each slide was quantified with a laser scanner (Molecular Dynamics, Sunnyvale, Calif.). The images were analyzed with ArrayVision 4.0 software (Imaging Research, Inc., Ontario, Canada). The raw fluorescent intensity for each spot was adjusted by subtracting the background. These readings were exported to a spreadsheet for further analysis.

### Example 4

### Comparison of Gene Expression Levels in the Entner Douderoff Pathway as Compared with the Embeden Meyerof Pathway

This Example presents microarray evidence demonstrating the use of the Embden-Meyerhoff pathway for carbon metabolism in the 16a strain.

FIG. 2 shows the relative levels of expression of genes for the Entner-Douderoff pathway and the Embden-Meyerhoff pathway. The relative transcriptional activity of each gene was estimated with DNA microarray as described previously (Example 3; Wei, et al., J. Bact. 183:545-556 (2001)).

Specifically, a single DNA microarray containing 4000 ORFs (open reading frames) of Methylomonas 16a was hybridized with probes generated from genomic DNA and total RNA. The genomic DNA of 16a was labeled with the Klenow fragment of DNA polymerase and fluorescent dye Cy-5, while the total RNA was labeled with reverse transcriptase and Cy-3. After hybridization, the signal intensities of both Cy-3 and Cy-5 for each spot in the array were quantified. The intensity ratio of Cy-3 and Cy-5 was then used to calculate the fraction of each transcript (as a percentage), according to the following formula: (gene ratio/sum of all ratio)×100. The value obtained reflects the relative abundance of mRNA of an individual gene. Accordingly, transcriptional activity of all the genes represented by the array can be ranked based on its relative mRNA abundance in a descending order. The numbers in FIG. 2 next to each step indicate the relative expression level of that enzyme. For example, mRNA abundance for the methane monooxygenase was the most highly expressed enzyme in the cell (ranked #1) because its genes had the highest transcriptional activity when the organism was grown with methane as the carbon source (FIG. 2). The next most highly expressed enzyme is methanol dehydrogenase (ranked #2). The hexulose-monophosphate synthase gene is one of the ten most highly expressed genes in cells grown on methane.

The genes considered “diagnostic” for Entner-Douderoff pathway are the 6-phosphogluconate dehydratase and the 2 keto-3-deoxy-6-phosphogluconate aldolase. In contrast, the phosphofructokinase and fructose bisphosphate aldolase are “diagnostic” of the Embden-Meyerhoff sequence. Messenger RNA transcripts of phosphofructokinase (ranked #232) and fructose bisphosphate aldolase (ranked #65) were in higher abundance than those for glucose 6 phosphate dehydrogenase (ranked #717), 6 phosphogluconate dehydratase (ranked #763) or the 2-keto-3-deoxy-6-gluconate aldolase. The data suggests that the Embden-Meyerhoff pathway enzymes are more strongly expressed than the Entner-Douderoff pathway enzymes. This result is surprising and counter to existing beliefs on the central metabolism of methanotrophic bacteria (Dijkhuizen, L., et al. The physiology and biochemistry of aerobic methanol-utilizing gram-negative and gram-positive bacteria. In: Methane and Methanol Utilizers, Biotechnology Handbooks 5. 1992. Eds: Colin Murrell, Howard Dalton; pp 149-157).

### Example 5

### Direct Enzymatic Evidence for a Pyrophosphate-Linked Phosphofructokinase

This example shows the evidence for the presence of a pyrophosphate-linked phosphofructokinase enzyme in the current strain, thereby confirming the functionality of the Embden-Meyerhoff pathway in the present Methylomonas strain.

Phosphofructokinase activity was shown to be present in Methylomonas 16a by using the coupled enzyme assay described below. Assay conditions are given in Table 7 below.

Coupled Assay Reactions

Phosphofructokinase reaction is measured by a coupled enzyme assay. Phosphofructokinase reaction is coupled with fructose 1,6, biphosphate aldolase followed by triosephosphate isomerase. The enzyme activity is measured by the disappearance of NADH.

Specifically, the enzyme phosphofructokinase catalyzes the key reaction converting fructose 6 phosphate and pyrophosphate to fructose 1,6 bisphosphate and orthophosphate. Fructose-1,6-bisphosphate is cleaved to 3-phosphoglyceraldehyde and dihydroxyacetonephosphate by fructose 1,6-bisphosphate aldolase. Dihydroxyacetonephosphate is isomerized to 3-phosphoglyceraldehyde by triosephosphate isomerase. Glycerol phosphate dehydrogenase plus NADH and 3-phosphoglyceraldehyde yields the alcohol glycerol-3-phosphate and NAD. Disappearance of NADH is monitored at 340 nm using spectrophotometer (UltraSpec 4000, Pharmacia Biotech).

TABLE 7
Assay Protocol

| | | Volume (μl) per | Final assay |
|---|---|---|---|
| | Stock solution | 1 mL total | concentration |
| Reagent | (mM) | reaction volume | (mM) |
| Tris-HCl pH 7.5 | 1000 | 100 | 100 |
| MgCl₂.2 H₂O | 100 | 35 | 3.5 |
| Na₄P₂O₇.10H₂O | 100 | 20 | 2 |
| or ATP | | | |
| Fructose-6- | 100 | 20 | 2 |
| phophate | | | |
| NADH | 50 | 6 | 0.3 |
| Fructose | 100 (units/mL) | 20 | 2 (units) |
| bisphosphate | | | |
| aldolase | | | |
| Triose phosphate | (7.2 units/μl) | 3.69 | 27 units |
| isomerase/glycerol | (0.5 units/μl) | | 1.8 units |
| phosphate | | | |
| dehydrogenase | | | |
| KCl | 1000 | 50 | 50 |
| H2O | | adjust to 1 mL | |
| Crude extract | | 0-50 | |

This coupled enzyme assay was further used to assay the activity in a number of other methanotrophic bacteria as shown below in Table 8. The data in Table 8 shows known ATCC strains tested for phosphofructokinase activity with ATP or pyrophosphate as the phosphoryl donor. These organisms were classified as either a Type I or Type X ribulose monophosphate-utilizing strains or a Type II serine utilizer. Established literature makes these types of classifications based on the mode of carbon incorporation, morphology, % GC content and the presence or absence of key specific enzymes in the organism.

TABLE 8
Comparison Of Pyrophosphate Linked And ATP Linked
Phosphofructokinase Activity In Different Methanotrophic Bacteria

| | | | ATP-PFK | Ppi-PFK |
|---|---|---|---|---|
| | | | umol | umol |
| | | Assimilation | NADH/ | NADH/ |
| Strain | Type | Pathway | min/mg | min/mg |
| Methylomonas 16a | i | Ribulose | 0 | 2.8 |
| ATCC PTA 2402 | | monophosphate | | |
| Methylomonas | I | Ribulose | 0.01 | 3.5 |
| agile | | monophosphate | | |
| ATCC 35068 | | | | |
| Methylobacter | I | Ribulose | 0.01 | 0.025 |
| whittenbury | | monophosphate | | |
| ATCC 51738 | | | | |
| Methylomonas | I | Ribulose | 0 | 0.3 |
| clara | | monophosphate | | |
| ATCC 31226 | | | | |
| Methylomicrobium | I | Ribulose | 0.02 | 3.6 |
| albus | | monophosphate | | |
| ATCC 33003 | | | | |
| Methylococcus | X | Ribulose | 0.01 | 0.04 |
| capsulatus | | monophosphate | | |
| ATCC 19069 | | | | |
| Methylosinus | II | Serine | 0.07 | 0.4 |
| sporium | | | | |
| ATCC 35069 | | | | |

Several conclusions may be drawn from the data presented above. First, it is clear that ATP (which is the typical phosphoryl donor for phosphofructokinase) is essentially ineffective in the phosphofructokinase reaction in methanotrophic bacteria. Only inorganic pyrophosphate was found to support the reaction in all methanotrophs tested. Secondly, not all methanotrophs contain this activity. The activity was essentially absent in Methylobacter whittenbury and in Methylococcus capsulatus. Intermediate levels of activity were found in Methylomonas clara and Methylosinus sporium. These data show that many methanotrophic bacteria may contain a hitherto unreported phosphofructokinase activity. It may be inferred from this that methanotrophs containing this activity have an active Embden-Meyerhoff pathway.

### Example 6

### Cloning of Carotenoid Genes from Pantoea stewartii

Primers were designed using the sequence from Pantoea ananatis to amplify a fragment by PCR containing a crt cluster of genes. These sequences included 5′-3′:

| | |
|---|---|
| ATGACGGTCTGCGCAAAAAAACACG | SEQ ID NO:43 |
| GAGAAATTATGTTGTGGATTTGGAATGC | SEQ ID NO:44 |

Chromosomal DNA was purified from Pantoea stewartii (ATCC no. 8199) and Pfu Turbo polymerase (Stratagene, La Jolla, Calif.) Was used in a PCR amplifcation reaction under the following conditions: 94° C., 5 min; 94° C. (1 min)-60° C. (1 min)-72° C. (10 min) for 25 cycles, and 72° C. for 10 min. A single product of approximately 6.5 kb was observed following gel electrophoresis. Taq polymerase (Perkin Elmer) was used in a ten min 72° C. reaction to add additional 3′ adenoside nucleotides to the fragment for TOPO cloning into pCR4-TOPO (Invitrogen, Carlsbad, Calif.). Following transformation to E. coli DH5α (Life Technologies, Rockville, Md.) by electroporation, several colonies appeared to be bright yellow in color, indicating that they were producing a carotenoid compound. Following plasmid isolation as instructed by the manufacturer using the Qiagen (Valencia, Calif.) miniprep kit, the plasmid containing the 6.5 kb amplified fragment was transposed with pGPS1.1 using the GPS-1 Genome Priming System kit (New England Biolabs, Inc., Beverly, Mass.). A number of these transposed plasmids were sequenced from each end of the transposon. Sequence was generated on an ABI Automatic sequencer using dye terminator technology (U.S. Pat. No. 5,366,860; EP 272007) using transposon specific primers. Sequence assembly was performed with the Sequencher program (Gene Codes Corp., Ann Arbor Mich.).

### Example 7

### Cloning of Rhodococcus erythropolis crtO

The present example describes the isolation, sequencing, and identification of a carotenoid biosynthetic pathway gene from Rhodococcus erythropolis AN12.

Isolation and Characterization of Strain AN12

Strain AN12 of Rhodococcus erythropolis was isolatd on the basis of being able to grow on aniline as the sole source of carbon and energy. Bacteria that grew on aniline were isolated from an enrichment culture. The enrichment culture was established by inoculating 1 ml of activated sludge into 10 ml of S12 medium (10 mM ammonium sulfate, 50 mM potassium phosphate buffer (pH 7.0), 2 mM MgCl₂, 0.7 mM CaCl₂, 50 μM MnCl₂, 1 μM FeCl₃, 1 μM ZnCl₃, 1.72 μM CuSO₄, 2.53 μM CoCl₂, 2.42 μM Na₂MoO₂, and 0.0001% FeSO₄) in a 125 ml screw cap Erlenmeyer flask. The activated sludge was obtained from a wastewater treatment facility. The enrichment culture was supplemented with 100 ppm aniline added directly to the culture medium and was incubated at 25° C. with reciprocal shaking. The enrichment culture was maintained by adding 100 ppm of aniline every 2-3 days. The culture was diluted every 14 days by replacing 9.9 ml of the culture with the same volume of S12 medium. Bacteria that utilized aniline as a sole source of carbon and energy were isolated by spreading samples of the enrichment culture onto S12 agar. Aniline (5 μL) was placed on the interior of each petri dish lid. The petri dishes were sealed with parafilm and incubated upside down at room temperature (approximately 25° C.). Representative bacterial colonies were then tested for the ability to use aniline as a sole source of carbon and energy. Colonies were transferred from the original S12 agar plates used for initial isolation to new S12 agar plates and supplied with aniline on the interior of each petri dish lid. The petri dishes were sealed with parafilm and incubated upside down at room temperature (approximately 25° C.).

The 16S rRNA genes of each isolate were amplified by PCR and analyzed as follows. Each isolate was grown on R2A agar (Difco Laboratories, Bedford, Mass.). Several colonies from a culture plate were suspended in 100 μl of water. The mixture was frozen and then thawed once. The 16S rRNA gene sequences were amplified by PCR using a commercial kit according to the manufacturer's instructions (Perkin Elmer) with primers HK12 (5′-GAGTTTGATCCTGGCTCAG-3′) (SEQ ID NO:45) and HK13 (5′-TACCTTGTTACGACTT-3′) (SEQ ID NO:46). PCR was performed in a Perkin Elmer GeneAmp 9600 (Norwalk, Conn.). The samples were incubated for 5 min at 94° C. and then cycled 35 times at 94° C. for 30 sec, 55° C. for 1 min, and 72° C. for 1 min. The amplified 16S rRNA genes were purified using a commercial kit according to the manufacturer's instructions (QIAquick PCR Purification Kit, Qiagen, Valencia, Calif.) and sequenced on an automated ABI sequencer. The sequencing reactions were initiated with primers HK12, HK13, and HK14 (5′-GTGCCAGCAGYMGCGGT-3′) (SEQ ID NO:47, where Y=C or T, M=A or C). The 16S rRNA gene sequence of each isolate was used as the query sequence for a BLAST search (Altschul, et al., Nucleic Acids Res. 25:3389-3402(1997)) of GenBank for similar sequences.

A 16S rRNA gene of strain AN12 was sequenced and compared to other 16S rRNA sequences in the GenBank sequence database. The 16S rRNA gene sequence from strain AN12 was at least 98% similar to the 16S rRNA gene sequences of high G+C Gram positive bacteria belonging to the genus Rhodococcus.

### Preparation of Genomic DNA for Sequencing and Sequence Generation

Genomic DNA preparation. Rhodococcus erythropolis AN12 was grown in 25 mL NBYE medium (0.8% nutrient broth, 0.5% yeast extract, 0.05% Tween 80) till mid-log phase at 37° C. with aeration. Bacterial cells were centrifuged at 4,000 g for 30 min at 4° C. The cell pellet was washed once with 20 ml 50 mM Na₂CO₃ containing 1M KCl (pH 10) and then with 20 ml 50 mM NaOAc (pH 5). The cell pellet was gently resuspended in 5 ml of 50 mM Tris-10 mM EDTA (pH 8) and lysozyme was added to a final concentration of 2 mg/mL. The suspension was incubated at 37° C. for 2 h. Sodium dodecyl sulfate was then added to a final concentration of 1% and proteinase K was added to 100 μg/ml final concentration. The suspension was incubated at 55° C. for 5 h. The suspension became clear and the clear lysate was extracted with equal volume of phenol:chloroform:isoamyl alcohol (25:24:1). After centrifuging at 17,000 g for 20 min, the aqueous phase was carefully removed and transferred to a new tube. Two volumes of ethanol were added and the DNA was gently spooled with a sealed glass pasteur pipet. The DNA was dipped into a tube containing 70% ethanol, then air dried. After air drying, DNA was resuspended in 400 μl of TE (10 mM Tris-1 mM EDTA, pH 8) with RNaseA (100 μg/mL) and stored at 4° C.

Library construction. 200 to 500 μg of chromosomal DNA was resuspended in a solution of 300 mM sodium acetate, 10 mM Tris-HCl, 1 mM Na-EDTA, and 30% glycerol, and sheared at 12 psi for 60 sec in an Aeromist Downdraft Nebulizer chamber (IBI Medical products, Chicago, Ill.). The DNA was precipitated, resuspended and treated with Bal31 nuclease (New England Biolabs, Beverly, Mass.). After size fractionation by 0.8% agarose gel electrophoresis, a fraction (2.0 kb, or 5.0 kb) was excised, cleaned and a two-step ligation procedure was used to produce a high titer library with greater than 99% single inserts.

Sequencing. A shotgun sequencing strategy approach was adopted for the sequencing of the whole microbial genome (Fleischmann, Robert et al., Whole-Genome Random sequencing and assembly of Haemophilus influenzae Rd Science, 269:1995).

Sequence was generated on an ABI Automatic sequencer using dye terminator technology (U.S. Pat. No. 5,366,860; EP 272007) using a combination of vector and insert-specific primers. Sequence editing was performed in either DNAStar (DNA Star Inc., Madison, Wis.) or the Wisconsin GCG program (Wisconsin Package Version 9.0, Genetics Computer Group (GCG), Madison, Wis.) and the CONSED package (version 7.0). All sequences represent coverage at least two times in both directions.

Sequence Analysis of CrtO

Two ORFs were identified in the genomic sequence of Rhodococcus erythropolis AN12 which shared homology to two different phytoene dehydrogenases. One ORF was designated CrtI and had the highest homology (45% identity, 56% similarity) to a putative phytoene dehydrogenase from Streptomyces coelicolor A3(2). The other ORF (originally designated as CrtI2, now as CrtO) had the highest homology (35% identity, 50% similarity; White O. et al Science 286 (5444), 1571-1577 (1999)) to a probable phytoene dehydrogenase DR0093 from Deinococcus radiodurans. Subsequent examination of the protein by motif analysis indicated that the crtO might function as a ketolase.

In Vitro Assay for Ketolase Activity of Rhodococcus CrtO

To confirm if crtO encoded a ketolase, the Rhodococcus crtO gene in E. coli was expressed was assayed for the presence of ketolase activity in vitro. The crtO gene was amplified from AN12 using the primers crtI2-N: ATGAGCGCATTTCTCGACGCC (SEQ ID NO:48) and crtI2-C: TCACGACCTGCTCGAACGAC (SEQ ID NO:49). The amplified 1599 bp full-length crtO gene was cloned into pTrcHis2-TOPO cloning vector (Invitrogen, Carlsbad, Calif.) and transformed into TOP10 cells following manufacture's instructions. The construct (designated pDCQ117) containing the crtO gene cloned in the forward orientation respective to the trc promoter on the vector was confirmed by restriction analysis and sequencing.

The in vitro enzyme assay was performed using crude cell extract from E. coli TOP10 (pDCQ117) cells expressing crtO. 100 ml of LB medium containing 100 μg/ml ampicillin was inoculated with 1 ml fresh overnight culture of TOP10 (pDCQ117) cells. Cells were grown at 37° C. with shaking at 300 rpm until OD₆₀₀ reached 0.6. Cells were then induced with 0.1 mM IPTG and continued growing for additional 3 hrs. Cell pellets harvested from 50 ml culture by centrifugation (4000 g, 15 min) were frozen and thawed once, and resuspended in 2 ml ice cold 50 mM Tris HCl (pH 7.5) containing 0.25% TritonX-100. 10 μg of β-carotene substrate (Spectrum Laboratory Products, Inc.) in 50 μl of acetone was added to the suspension and mixed by pipetting. The mixture was divided into two tubes and 250 mg of zirconia/silica beads (0.1 mm, BioSpec Products, Inc, Bartlesville, Okla.) was added to each tube. Cells were broken by bead beating for 2 min, and cell debris was removed by spinning at 10000 rpm for 2 min in an Eppendorf microcentrifuge 5414C. The combined supernatant (2 ml) was diluted with 3 ml of 50 mM Tris pH 7.5 buffer in a 50 ml flask, and the reaction mixture was incubated at 30° C. with shaking at 150 rpm for different lengths of time. The reaction was stopped by addition of 5 ml methanol and extraction with 5 ml diethyl ether. 500 mg of NaCl was added to separate the two phases for extraction. Carotenoids in the upper diethyl ether phase was collected and dried under nitrogen. The carotenoids were re-dissolved in 0.5 ml of methanol, for HLPC analysis, using a Beckman System Gold® HPLC with Beckman Gold Nouveau Software (Columbia, Md.). 0.1 ml of the crude acetone extraction was loaded onto a 125×4 mm RP8 (5 μm particles) column with corresponding guard column (Hewlett-Packard, San Fernando, Calif.). The flow rate was 1 ml/min and the Solvent program was 0-11.5 min 40% water/60% methanol, 11.5-20 min 100% methanol, 20-30 min 40% water/60% methanol. Spectral data was collected using a Beckman photodiode array detector (model 168).

Three peaks were identified at 470 nm in the 16 hr reaction mixture. When compared to standards, it was determined that the peak with a retention time of 15.8 min was β-carotene and the peak with retention time of 13.8 min was canthaxanthin. The peak at 14.8 min was most likely echinenone, the intermediate with only one ketone group addition. In the 2 hr reaction mixture, the echinenone intermediate was the only reaction product and no canthaxanthin was produced. Longer incubation times resulted in higher levels of echinenone and the appearance of a peak corresponding to canthaxanthin. Canthaxanthin is the final product in this step representing the addition of two ketone groups (Table 9). To confirm that the ketolase activity was specific for crtO gene, the assay was also performed with extracts of control cells that would not use 1-carotene as the substrate. No product peaks were detected in the control reaction mixture.

In summary, the in vitro assay data confirmed that crtO encodes a ketolase, which converted β-carotene into canthaxanthin (two ketone groups) via echinenone (one ketone group) as the intermediate. This symmetric ketolase activity of Rhodococcus CrtO is different from what was reported for the asymmetric function of Synechocystis CrtO.

TABLE 9
HPLC Analysis Of The In Vitro Reaction Mixtures With
Rhodococcus CrtO

| | Canthaxanthin | Echinenone | β-carotene |
|---|---|---|---|
| | 474 nm | 459 nm | 449 nm 474 nm |
| | 13.8 min | 14.8 min | 15.8 min |
| 0 hr | 0% | 0% | 100% |
| 2 hr | 0% | 14% | 86% |
| 16 hr | 16% | 28% | 56% |
| 20 hr | 30% | 35% | 35% |

### Example 8

All sequences from Examples 6 and 7 were identified by conducting BLAST (Basic Local Alignment Search Tool; Altschul, S. F., et al., (1993) J. Mol. Biol. 215:403-410; see also www.ncbi.nlm.nih.gov/BLAST/) searches for similarity to sequences contained in the BLAST “nr” database, according to the methodology of Example 2.

The results of these BLAST comparisons are given below in Table 10. The table displays data based on the BLASTXnr algorithm with values reported in expect values. The Expect value estimates the statistical significance of the match, specifying the number of matches, with a given score, that are expected in a search of a database of this size absolutely by chance.

TABLE 10
Identification of Carotenoid Genes Based on Sequence Homology

| | | | SEQ ID | | | | |
|---|---|---|---|---|---|---|---|
| Gene Name | Similarity Identified | SEQ ID | Peptide | % Identity^{a} | % Similarity^{b} | E-value^{c} | Citation |
| crtE | Geranylgeranyl pryophosphate | 25 | 26 | 83 | 88 | e−137 | Misawa et al., J. Bacteriol. 172 |
| | synthetase (or GGPP synthetase, | | | | | | (12), 6704-6712 (1990) |
| | or farnesyltranstransferase) | | | | | | |
| | EC 2.5.1.29 | | | | | | |
| | gi\|117509\|sp\|P21684\|CRTE_PAN | | | | | | |
| | AN GERANYLGERANYL | | | | | | |
| | PYROPHOSPHATE | | | | | | |
| | SYNTHETASE (GGPP | | | | | | |
| | SYNTHETASE) | | | | | | |
| | (FARNESYLTRANSTRANSFERASE) | | | | | | |
| crtX | Zeaxanthin glucosyl transferase | 27 | 28 | 75 | 79 | 0.0 | Lin et al., Mol. Gen. Genet. |
| | EC 2.4.1.— | | | | | | 245 (4), 417-423 (1994) |
| | gi\|1073294\|pir\|\|S52583 crtX | | | | | | |
| | protein —Erwinia herbicola | | | | | | |
| crtY | Lycopene cyclase | 29 | 30 | 83 | 91 | 0.0 | Lin et al., Mol. Gen. Genet. |
| | | | | | | | 245 (4), 417-423 (1994) |
| | gi\|1073295\|pir\|\|S52585 dycopene | | | | | | |
| | cyclase —Erwinia herbicola | | | | | | |
| crtI | Phytoene desaturaseEC 1.3.—.— | 31 | 32 | 89 | 91 | 0.0 | Lin et al., Mol. Gen. Genet. |
| | | | | | | | 245 (4), 417-423 (1994) |
| | gi\|1073299\|pir\|\|S52586 phytoene | | | | | | |
| | dehydrogenase (EC 1.3.—.—) — | | | | | | |
| | Erwinia herbicola | | | | | | |
| crtB | Phytoene synthaseEC2.5.1.— | 33 | 34 | 88 | 92 | e−150 | Lin et al., Mol. Gen. Genet. |
| | | | | | | | 245 (4), 417-423 (1994) |
| | gi\|1073300\|pir\|\|S52587 | | | | | | |
| | prephytoene pyrophosphate | | | | | | |
| | synthase —Erwinia herbicola | | | | | | |
| crtZ | -carotene hydroxylase | 35 | 36 | 88 | 91 | 3e−88 | Misawa et al., J. Bacteriol. 172 |
| | | | | | | | (12), 6704-6712 (1990) |
| | gi\|117526\|sp\|P21688\|CRTZ_PAN | | | | | | |
| | AN -CAROTENE | | | | | | |
| | HYDROXYLASE | | | | | | |
| crtO | slr0088 —Synechocystis | 37 | 38 | 35 | 64% | | White O. et al Science 286 |
| | hypothetical protein | | | | | | (5444), 1571-1577 (1999) |
| | | | | | | | Fernández-González, et al., J. |
| | | | | | | | Biol. Chem., 1997, 272: |
| | | | | | | | 9728-9733 |

### Example 9

### Expression of β-carotene in Methylomonas 16A Growing on Methane

The crt gene cluster comprising the crtEXYIBZ genes from Pantoea stewartii (Example 6) was introduced into Methylomonas 16a to enable the synthesis of desirable 40-carbon carotenoids.

Primers were designed using the sequence from Erwinia uredovora to amplify a fragment by PCR containing the crt genes. These sequences included 5′-3′:

| | |
|---|---|
| ATGACGGTCTGCGCAAAAAAACACG | SEQ ID 43 |
| GAGAAATTATGTTGTGGATTTGGAATGC | SEQ ID 44 |

Chromosomal DNA was purified from Pantoea stewartii (ATCC no. 8199) and Pfu Turbo polymerase (Stratagene, La Jolla, Calif.) was used in a PCR amplifcation reaction under the following conditions: 94° C., 5 min; 94° C. (1 min)-60° C. (1 min)-72° C. (10 min) for 25 cycles, and 72° C. for 10 min. A single product of approximately 6.5 kb was observed following gel electrophoresis. Taq polymerase (Perkin Elmer) was used in a ten minute 72° C. reaction to add additional 3′ adenoside nucleotides to the fragment for TOPO cloning into pCR4-TOPO (Invitrogen, Carlsbad, Calif.). Following transformation to E. coli DH5α (Life Technologies, Rockville, Md.) by electroproation, several colonies appeared to be bright yellow in color indicating that they were producing a carotenoid compound

For introduction into Methylomonas 16a, the crt gene cluster from pCR4-crt was first subcloned into the unique EcoRI site within the chloramphenicol-resistance gene of the broad host range vector, pBHR1 (MoBiTec, LLC, Marco Island, Fla.). pBHR1 (500 ng) was linearized by digestion with EcoRI (New England Biolabs, Beverly, Mass.) and then dephosphorylated with calf intestinal alkaline phosphatase (Gibco/BRL, Rockville, Md.). pCR4-crt was digested with EcoRI and the 6.3 kb EcoRI fragment containing the crt gene cluster (crtEXYIB) was purified following gel electrophoresis in 0.8% agarose (TAE). This DNA fragment was ligated to EcoRI-digested pBHR1 and the ligated DNA was used to transform E. coli DH5α by electroporation. Transformants were selected on LB medium containing 50 ug/ml kanamycin.

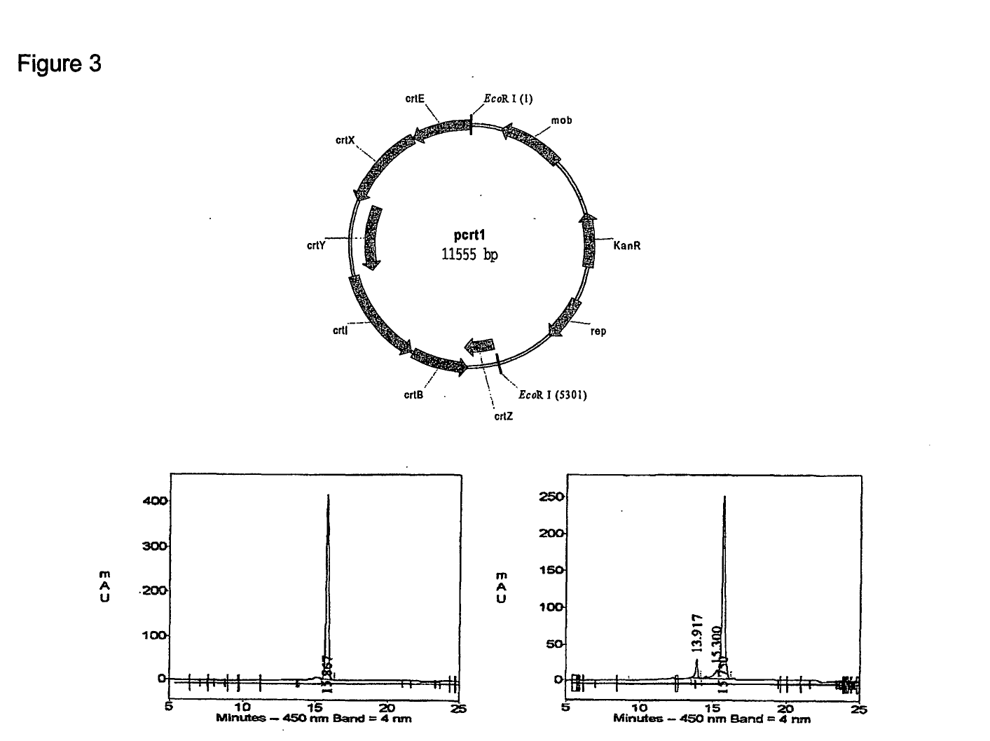

Several isolates were found to be sensitive to chloramphenicol (25 ug/ml) and demonstrated a yellow colony phenotype after overnight incubation at 37° C. Analysis of the plasmid DNA from these transformants confirmed the presence of the crt gene cluster cloned in the same orientation as the pBHR1 chloramphenicol-resistance gene and this plasmid was designated pCrt1 (FIG. 3). In contrast, analysis of the plasmid DNA from transformants demonstrating a white colony phenotype confirmed the presence of the crt gene cluster cloned in the opposite orientation as the pBHR1 chloramphenicol-resistance gene and this plasmid was designated pCrt2. These results suggested that functional expression of the crt gene cluster was directed from the pBHR1 cat promoter.

Plasmid pCrt1 was transferred into Methylomonas 16a by tri-parental conjugal mating. The E. coli helper strain containing pRK2013 and the E. coli DH5α donor strain containing pCrt1 were grown overnight in LB medium containing kanamycin (50 μg/mL), washed three times in LB, and resuspended in a volume of LB representing approximately a 60-fold concentration of the original culture volume. The Methylomonas 16a recipient was grown for 48 hours in Nitrate liquid “BTZ-3” medium (General Methods) in an atmosphere containing 25% (v/v) methane, washed three times in BTZ-3, and resuspended in a volume of BTZ-3 representing a 150-fold concentration of the original culture volume. The donor, helper, and recipient cell pastes were combined on the surface of BTZ-3 agar plates containing 0.5% (w/v) yeast extract in ratios of 1:1:2 respectively. Plates were maintained at 30° C. in 25% methane for 16-72 hours to allow conjugation to occur, after which the cell pastes were collected and resuspended in BTZ-3. Dilutions were plated on BTZ-3 agar containing kanamycin (50 μg/mL) and incubated at 30° C. in 25% methane for up to 1 week. Transconjugants were streaked onto BTZ-3 agar with kanamycin (50 μg/mL) for isolation. Analysis of plasmid DNA isolated from these transconjugants confirmed the presence of pCrt1 (FIG. 3).

For analysis of carotenoid composition, transconjugants were cultured in 25 ml BTZ-3 containing kanamycin (50 μg/mL) and incubated at 30° C. in 25% methane as the sole carbon source for up to 1 week. The cells were harvested by centrifugation and frozen at −20° C. After thawing, the pellets were extracted and carotenoid content was analyzed by HPLC according to the methodology of the General Methods.

HPLC analysis of extracts from Methylomonas 16a containing pCrt1 confirmed the synthesis of β-carotene. The left panel of FIG. 3 shows the HPLC results obtained using the β-carotene standard and a single peak is present at 15.867 min. Similarly, the right panel of FIG. 3 shows the HPLC profile obtained for analysis of Methylomonas 16a transconjugant cultures containing the pCrt1 plasmid. A similar peak at 15.750 min is indicative of β-carotene in the cultures.

### Example 10

### Expression of Zeaxanthin in Methylomonas 16A Growing on Methane

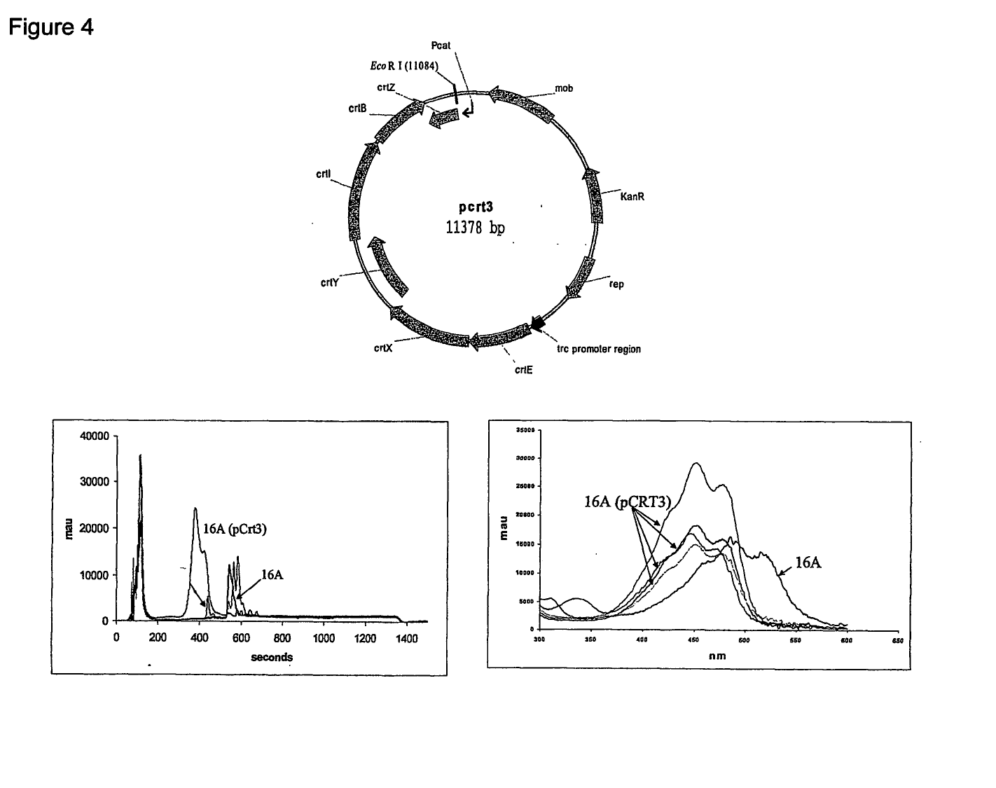

To enable the synthesis of zeaxanthin in Methylomonas 16a, the crt gene cluster from pTrcHis-crt2 (as described above) was subcloned into the chloramphenicol-resistance gene of the broad host range vector, pBHR1 (MoBiTec, LLC, Marco Island, Fla.). pBHR1 (500 ng) was digested sequentially with EcoRI and ScaI and the 4876 bp EcoRI-ScaI DNA fragment was purified following gel electrophoresis in 0.8% agarose (TAE). Plasmid pTrcHis-crt2 was digested simultaneously with SspI and EcoRI and the 6491 bp SspI-EcoRI DNA fragment containing the crt gene cluster (crtEXYIB) under the transcriptional control of the E. coli trc promoter was purified following gel electrophoresis in 0.8% agarose (TAE). The 6491 bp SspI-EcoRI fragment was ligated to the 4876 bp EcoRI-ScaI fragment and the ligated DNA was used to transform E coli DH5α by electroporation. Transformants were selected on LB medium containing 50 ug/ml kanamycin. Several kanamycin-resistant isolates were also sensitive to chloramphenicol (25 ug/ml) and demonstrated yellow colony color after overnight incubation at 37° C. Analysis of the plasmid DNA from these transformants confirmed the presence of the crt gene cluster cloned into pBHR1 under the transcriptional control of the E. coli trc promoter and were designated as pCrt3. The plasmid map for this pCrt3 construct is shown in FIG. 4. The p_{cat} promoter is illustrated with a small bold black arrow, in contrast to the large wide arrows, representing specific genes as labeled.

Plasmid pCrt3 was transferred into Methylomonas 16a by tri-parental conjugal mating, as described above for pCrt1 (Example 9). Transconjugants containing this plasmid demonstrated yellow colony color following growth on BTZ-3 agar with kanamycin (50 μg/mL) and methane as the sole carbon source.

HPLC analysis of extracts from Methylomonas 16A containing pCrt3 revealed the presence of zeaxanthin and its mono- and diglucosides. These results are shown in FIG. 4. The left panel shows the HPLC profile of extracts from Methylomonas 16A or Methylomonas 16A containing the pcrt3. The right panel shows the UV spectra of the individual peaks displayed in the HPLC profile and demonstrate the synthesis of zeaxanthin and its mono- and di-glucosides in Methylomonas 16A containing pcrt3. These results suggested that the crtEXYIB genes were functionally expressed from the trc promoter while the crtZ gene was transcribed in the opposite orientation from the pBHR1 cat promoter in Methylomonas 16A.

One skilled in the art would expect that deletion of crtX from this and subsequent plasmids should enable the production of zeaxanthin without formation of the mono- and di-glucosides. Furthermore, a plasmid in which the crtEYIBZ genes are expressed in the same orientation from one or more promoters may be expected to alleviate potential transcriptional interference and enhance the synthesis of zeaxanthin. This would readily be possible using standard cloning techniques know to those skilled in the art.

### Example 11

### Expression of Zeaxanthin in Methylomonas 16A Growing on Methane, with an Optimized HMPS Promoter

Analysis of gene array data following growth of Methylomonas 16a on methane suggested the hexoulose-monophosphate synthase (HMPS) to be one of the ten most highly expressed genes. Thus, one may use the DNA sequences comprising the HMPS promoter to direct high-level expression of heterologous genes, including those in the P. stewartii crt gene cluster, in Methylomonas 16A. Analysis of the 5′-DNA sequences upstream from the HMPS gene identified potential transcription initiation sites in both DNA strands using the NNPP/neural network prokaryotic promoter prediction program from Baylor College of Medicine Predictions concerning the forward strand of the H6P synthase are shown below in Table 11; similar results are shown below in Table 12 for the reverse strand.

TABLE 11
Promoter Predictions for H6P synthase-Forward Strand

| Start | End | Score | Promoter Sequence* | |
|---|---|---|---|---|
| 63 | 108 | 0.93 | GAGAATTGGCTGAAAAACCAAATAAATAACAAAATTTAG | (SEQ ID NO:50) |
| | | | CGAGTAAATGG | |
| 119 | 164 | 0.91 | TTCAATTGACAGGGGGGCTCGTTCTGATTTAGAGTTGCT | (SEQ ID NO:51) |
| | | | GCCAGCTTTTT | |
| 211 | 256 | 0.85 | GGGTTGTCCAGATGTTGGTGAGCGGTCCTTATAACTATA | (SEQ ID NO:52) |
| | | | ACTGTAACAAT | |

*The transcription start sites are indicated in bold text.

TABLE 12
Promoter Predictions for H6P synthase-Reverse Strand

| Start | End | Scor | Promoter S quence* | |
|---|---|---|---|---|
| 284 | 239 | 0.89 | TTAATGGTCTTGCCATGAGATGTGCTCCGATTGTTACAG | (SEQ ID NO:53) |
| | | | TTATAGTTATA | |
| 129 | 84 | 0.95 | CCCCCTGTCAATTGAAAGCCCGCCATTTACTCGCTAAAT | (SEQ ID NO:54) |
| | | | TTTGTTATTTA | |

*The transcription start sites are indicated in bold text.

Based on these sequences, the following primers were used in a polymerase chain reaction (PCR) to amplify a 240 bp DNA sequence comprising the HMPS promoter from Methylomonas 16a genomic DNA:

| | |
|---|---|
| 5′ CCGAGTACTGAAGCGGGTTTTTGCAGGGAG 3′ | (SEQ ID NO:39) |
| 5′ GGGCTAGCTGCTCCGATTGTTACAG 3′ | (SEQ ID NO:40) |

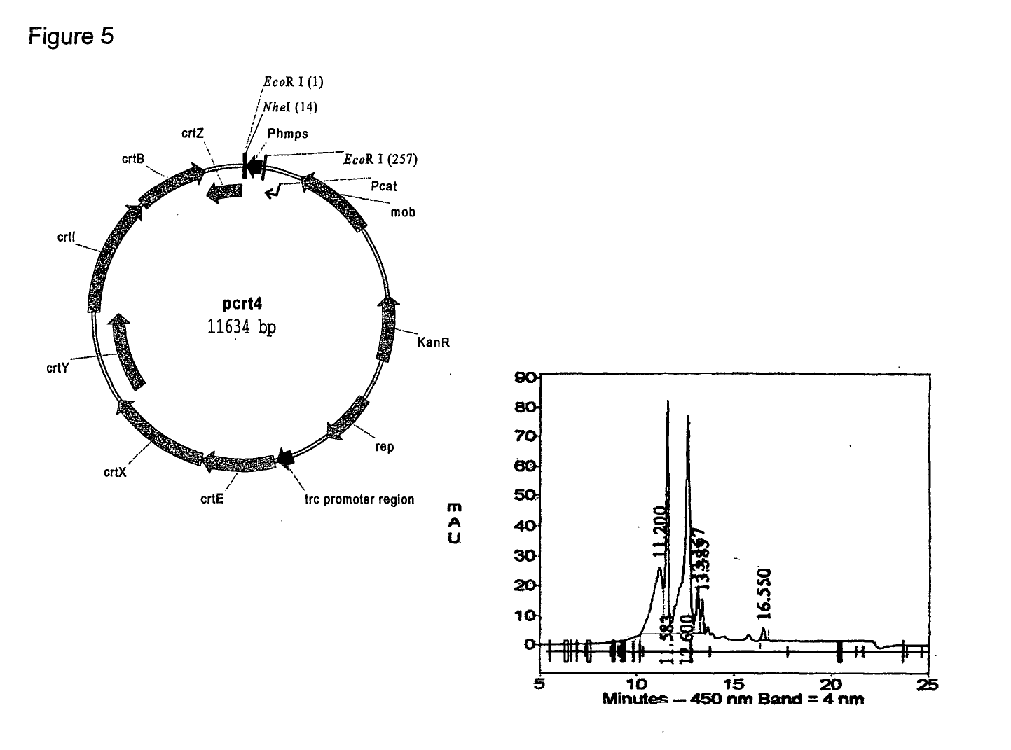

The PCR conditions were: 94° C. for 2 min, followed by 35 cycles of 94° C. for 1 min, 50° C. for 1 min and 72° C. for 2 min, and final extension at 72° C. for 5 min. After purification, the 240 bp PCR product was ligated to pCR2.1 (Invitrogen, Carlsbad, Calif.) and transformed into E. coli DH5α by electroporation. Analysis of the plasmid DNA from transformants that demonstrated white colony color on LB agar containing kanamycin (50 μg/ml) and X-gal identified the expected plasmid, which was designated pHMPS. PHMPS was digested with EcoRI and the 256 bp DNA fragment containing the HMPS promoter was purified following gel electrophoresis in 1.5% agarose (TEA). This DNA fragment was ligated to pCrt3 previously digested with EcoRI and dephosphorylated with calf intestinal alkaline phosphatase. The ligated DNA was used to transform E. coli DH5α by electroporation. Analysis of plasmid DNA from transformants that demonstrated yellow colony color on LB agar containing kanamycin (50 μg/ml) identified the expected plasmid, designated pCrt4, containing the crtEXYIB genes under the transcriptional control of the trc promoter and the crtZ gene under the transcriptional control of the hmps promoter (FIG. 5).

Plasmid pCrt4 was transferred into Methylomonas 16a by tri-parental conjugal mating. Transconjugants containing this plasmid demonstrated yellow colony color following growth on BTZ-3 agar with kanamycin (50 μg/mL) and methane as the sole carbon source. HPLC analysis of extracts from Methylomonas 16a containing pCrt4 revealed the presence of zeaxanthin, and its mono- and di-glucosides, thereby confirming expression of the crtZ gene. This data is shown in FIG. 5. Peaks with retention times of 13.38 min, 12.60 min and 11.58 min correspond to zeaxanthin, a mixture of zeaxanthin mono-glucosides and zeaxanthin diglucoside, respectively,

### Example 12

### Expression of Canthaxanthin and Astaxanthin in Methylomonas 16A Growing on Methane

To enable the synthesis of canthaxanthin and astaxanthin in Methylomonas 16a, the Rhodococcus erythropolis AN12 crtO gene encoding β-carotene ketolase (Example 7) was cloned into pcrt4. The crtO gene was amplified by PCR from pDCQ117 (Example 7) using the following primers to introduce convenient SpeI and NheI restriction sites as well as the ribosome binding site found upstream of crtE which was presumably recognized in Methylomonas 16a.

(SEQ ID NO:41

5′-AGCAGCTAGCGGAGGAATAAACCATGAGCGCATTTCTC-3′

(SEQ ID NO:42)

5′-GACTAGTCACGACCTGCTCGAACGAC-3′

The PCR conditions were: 95° C. for 5 min, 35 cycles of 95° C. for 30 sec, 45-60° C. gradient with 0.15° C. decrease/cycle for 30 sec and 72° C. for 90 sec, and a final extension at 72° C. for 7 min. The 1653 bp PCR product was purified following gel electrophoresis in 1.0% agarose (TAE), digested simultaneously with SpeI and NheI restriction endonucleases and then ligated to pCrt4 previously digested with NheI and dephosphorylated with calf intestinal alkaline phosphatase. The ligated DNA was used to transform E. coli DH5α by electroporation.

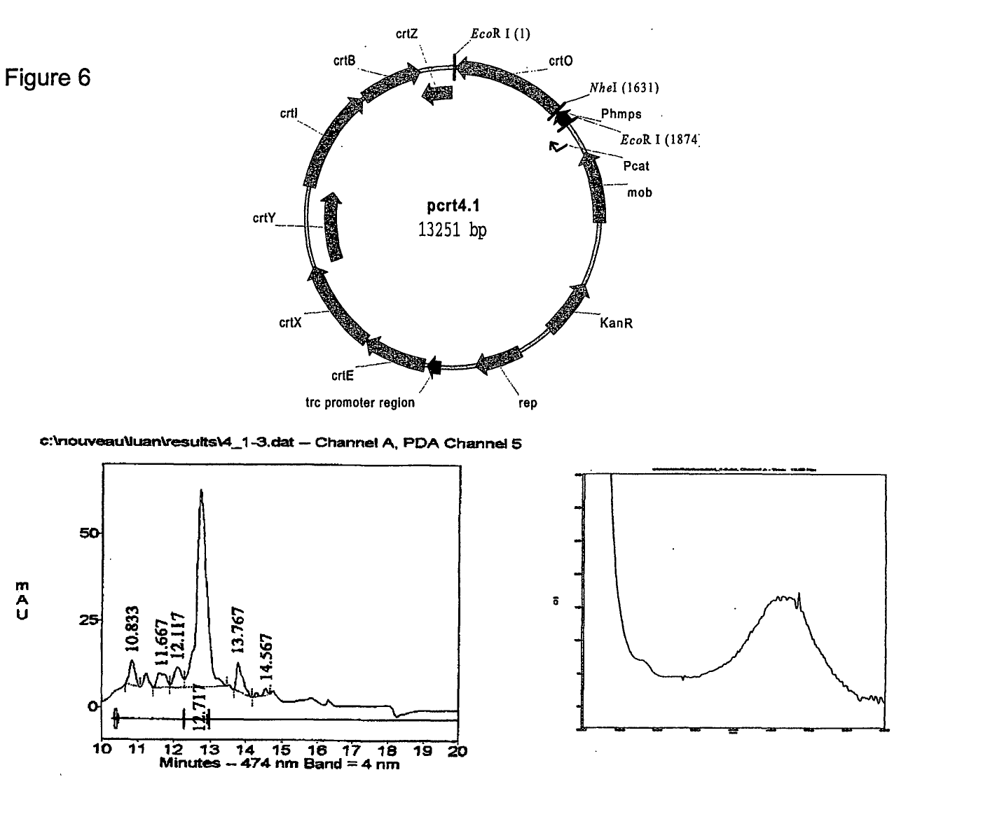

Analysis of plasmid DNA from transformants that demonstrated yellow colony color on LB agar containing kanamycin (50 ug/ml) identified the expected plasmid, designated pCrt4.1, in which the crtEXYIB genes were cloned under the transcriptional control of the trc promoter and the crtO and crtZ genes were cloned under the transcriptional control of the hmps promoter This plasmid construct is shown in FIG. 6. Upon prolonged incubation, transformants containing pcrt4.1 demonstrated a salmon pink colony color.

Plasmid pCrt4.1 was transferred into Methylomonas 16a by tri-parental conjugal mating. Transconjugants containing this plasmid demonstrated orange colony color following growth on BTZ-3 agar with kanamycin (50 μg/mL) and methane as the sole carbon source. HPLC analysis of extracts of Methylomonas 16a containing pCrt4.1 are shown in FIG. 6. These results revealed the presence of the endogenous Methylomonas 16a 30-carbon carotenoid (retention time of 12.717 min) as well as canthaxanthin (retention time of 13.767 min). The retention time of the wild-type pigment is very close to that expected for astaxanthin. Analysis of a shoulder on this peak confirmed the presence of astaxanthin

The predominant formation of the wild-type 16A pigment in this strain suggested transcriptional interference of the crtEXYIB operon by high-level expression of the crtOZ operon from the strong hmps promoter. In addition, it is hypothesized that the cat promoter on the pBHR1 vector may be directing expression of crtOZ in concert with the hmps promoter. Plasmids in which the crtEYIBZO genes are expressed in the same orientation from one or more promoters may be expected to alleviate potential transcriptional interference and thereby enhance the synthesis of canthaxanthin and astaxanthin.

### Example 13

### Enhanced Synthesis of the Native Carotenoid of Methylomonas 16A by Amplification of Upper Isoprenoid Pathway Genes

Native isoprene pathway genes dxs and dxr were amplified from the Methylomonas 16a genome by using PCR with the following primers.

Dxs Primers:

Forward reaction: aaggatccgcgtattcgtactc (contains a Bam HI site, SEQ ID NO:55).

Reverse reaction: ctggatccgatctagaaataggctcgagttgtcgttcagg (contains a Bam HI and a Xho I site, SEQ ID NO:56).

Dxr Primers:

Forward reaction: aaggatcctactcgagctgacatcagtgct (contains a Bam HI and a Xho I site, SEQ ID NO:57).

Reverse reaction: gctctagatgcaaccagaatcg (contains a Xba I site, SEQ ID NO:58).

The expected PCR products of dxs and dxr genes included sequences of 323 bp and 420 bp, respectively, upstream of the start codon of each gene in order to ensure that the natural promoters of the genes were present. The PCR program (in Perkin-Elmer. Norwalk, Conn.) was as follows: denaturing 95° C. (900 sec); 35 cycles of 94° C. (45 sec), 58° C. (45 sec), 72° C. (60 sec); final elongation 72° C. (600 sec). The reaction mixture (50 ul total volume) contained: 25 μl Hot Star master mix (Qiagen, Valencia, Calif.), 0.75 μl genomic DNA (approx. 0.1 ng), 1.2 μl sense primer (=10 pmol), 1.2 μl antisense primer (=10 pmol), 21.85 μl deionized water.

Standard procedures (Sambrook, J., Fritsch, E. F. and Maniatis, T. Molecular Cloning: A Laboratory Manual, Second Edition, Cold Spring Harbor Laboratory Press, Cold Spring Harbor (1989)), were used in order to clone dxs and dxr into pTJS75::lacZ:Tn5Kn, a low-copy, broad-host plasmid (Schmidhauser and Helinski J. Bacteriology. Vol. 164:446-455 (1985)).

For isolation, concentration, and purification of DNA, Qiagen kits (Valencia, Calif.) were used. Enzymes for the cloning were purchased from Gibco/BRL (Rockville, Md.) or NEB (Beverly, Mass.). To transfer plasmids into E. coli, One Shot Top10 competent cells (Invitrogen, Carlsbad, Calif.), cuvettes (0.2 cm; Invitrogen), and Bio-Rad Gene Pulser III (Hercules, Calif.) with standard settings were used for electroporation.

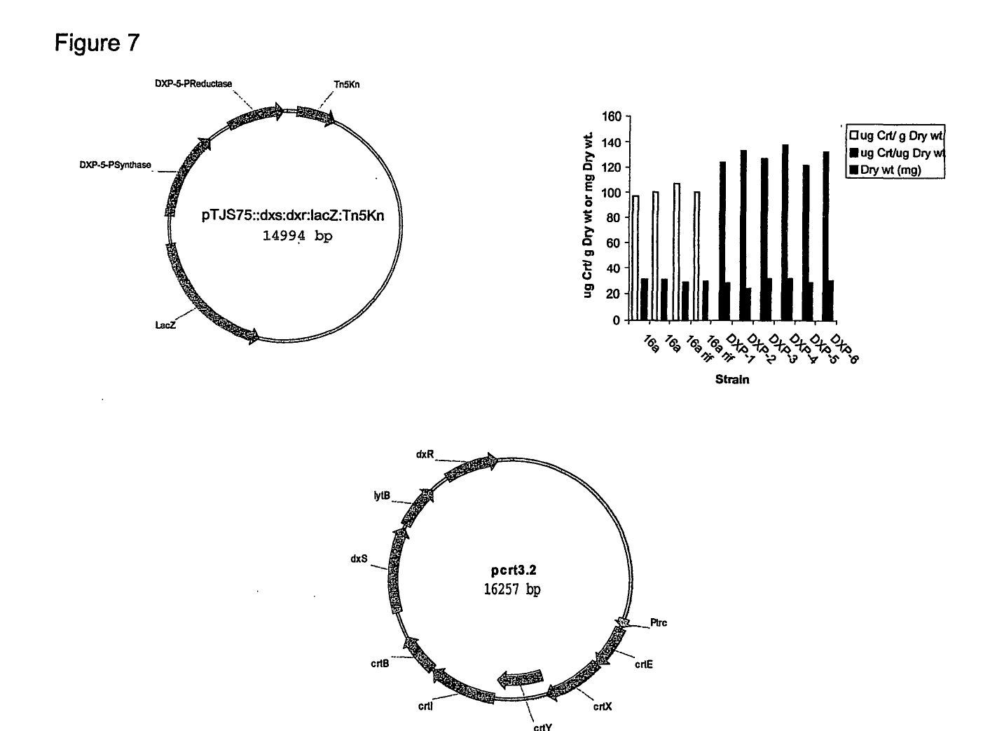

First, dxs was cloned into the Bam HI site, which was located between the lacZ gene and the Tn5Kn cassette of pTJS75::lacZ:Tn5Kn. The resulting plasmids were isolated from E. coli transformants growing on LB+ kanamycin (Kn, 50 μg/mL). The plasmid containing the insert in direction of the Kn-resistance gene (as confirmed by restriction analysis) was chosen for further cloning. The dxr gene was cloned in between dxs and the Tn5Kn cassette by using the Xho I and Xba I sites. The anticipated plasmid was isolated from E. coli transformants. The presence of dxs and dxr in the plasmid was confirmed by restriction analysis and sequencing. The resulting plasmid, pTJS75::dxs:dxr:lacZ:Tn5Kn is shown in FIG. 7

The plasmid pTJS75::dxs:dxr:lacZ:Tn5Kn was transferred from E. coli into Methylomonas 16a by triparental conjugation. A spontaneous rifampin (Rif)-resistant isolate of strain Methylomonas 16a was used as the recipient to speed the isolation of the methanotroph from contaminating E. coli following the mating. Six separately isolated kanamycin-resistant Methylomonas 16a transconjugants were used for carotenoid content determination.

For carotenoid determination, six 100 mL cultures of transconjugants (in BTZ+50 μg/mL Kn) were grown under methane (25%) over the weekend to stationary growth phase. Two cultures of each, the wild-type strain and its Rif-resistant derivative without the plasmid, served as a control to see whether there are different carotenoid contents in those strains and to get a standard deviation of the carotenoid determination. Cells were spun down, washed with distilled water, and freeze-dried (lyophilizer: Virtis, Gardiner, N.Y.) for 24 h in order to determine dry-weights. After the dry-weight of each culture, was determined, cells were extracted. First, cells were welled with 0.4 mL of water and let stand for 15 min. After 15 min, four mL of acetone was added and thoroughly vortexed to homogenize the sample. The samples were then shaken at 30° C. for 1 hr. After 1 hr, the cells were centrifuged. Pink coloration was observed in the supernatant. The supernatant was collected and pellets were extracted again with 0.3 mL of water and 3 mL of acetone. The supernatants from the second extraction were lighter pink in color. The supernatants of both extractions were combined, their volumes were measured, and analyzed spectrophotometrically. No qualitative differences were seen in the spectra between negative control and transconjugant samples. In acetone extract, a following observation was typical measured by spectrophotometer (shoulder at 460 nm, maxima at 491 and 522 nm) (Amersham Pharmacia Biotech, Piscataway, N.J.). For calculation of the carotenoid content, the absorption at 491 nm was read, the molar extinction coefficient of bacterioruberin (188,000) and a MW of 552 were used. The MW of the carotenoid (552 g/mol) was determined by MALDI-MS of a purified sample (Silica/Mg adsorption followed by Silica column chromatography, reference: Britton, G., Liaaen-Jensen, S., Pfander, H., Carotenoids Vol. 1a; Isolation and analysis, Birkhäuser Verlag, Basel, Boston, Berlin (1995)).

A crude acetone extract from Methylomonas 16a cells has a typical absorption spectrum (inflexion at 460 nm, maxima at 491 nm and 522 nm). HPLC analysis (as described in the General Methods, except solvent program: 0-10 min 15% water/85% methanol, then 100% methanol) of acetone extracts confirmed that one major carotenoid (net retention volume at about 6 mL) with the above mentioned absorption spectrum is responsible for the pink coloration of wild-type and transconjugant Methylomonas 16a cells. Because nothing else in the extract absorbs at 491 nm, carotenoid content was directly measured in the acetone extract with a spectrophotometer (Amersham Pharmacia Biotech, Piscataway, N.J.).

The molar extinction coefficient of bacterioruberin (188,000), was used for the calculation of the quantity. The following formula was used (Lambert-Beer's law) to determine the quantity of carotenoid:

Ca=A_{491 nm}/(d×ε×v×MW)

Ca: Carotenoid amount (g)

A_{491 nm}: Absorption of acetone extract at 491 nm (−)

d: Light path in cuvette (1 cm)

ε: Molar extinction coefficient (L/(mol×cm))

MW: Molecular weight (g/mol)

v: Volume of extract (L)

To get the carotenoid content, the calculated carotenoid amount has to be divided by the corresponding cell dry weight.

TABLE 13
Native Carotenoid contents in Methylomonas 16a cells

| | dry weight | | carotenoid content |
|---|---|---|---|
| Cultures | (mg) | carotenoid (g) | (μg/g) |
| 16a-1^{a} | 30.8 | 3.00194E−06 | 97.5 |
| 16a-2^{a} | 30.8 | 3.0865E−06 | 100.2 |
| 16a Rif-1^{b} | 29.2 | 3.12937E−06 | 107.2 |
| 16a Rif-2^{b} | 30.1 | 3.02014E−06 | 100.3 |
| dxp 1^{c} | 28.2 | 3.48817E−06 | 123.7 |
| dxp 2^{c} | 23.8 | 3.17224E−06 | 133.3 |
| dxp 3^{c} | 31.6 | 4.01962E−06 | 127.2 |
| dxp 4^{c} | 31.8 | 4.38899E−06 | 138.0 |
| dxp 5^{c} | 28.4 | 3.4547E−06 | 121.6 |
| dxp 6^{c} | 30.3 | 4.00817E−06 | 132.3 |

^{a}Methylomonas 16a native strain
^{b}Rif resistant derivative of Methylomonas 16a without plasmid
^{c}transconjugants containing pTJS75::dxs:dxr:lacZ:Tn5Kn plasmid

There were no significant differences between four negative controls. Likewise, there were no significant differences between six transconjugants. However, approximately 28% increase in average carotenoid production was observed in the transconjugants in comparison to the average carotenoid production in negative controls (Table 13; FIG. 7

In order to confirm the structure, Methylobacterium rhodinum (formerly Pseudomonas rhodos: ATCC No. 14821) of which C30-carotenoid was identified was used as a reference strain (Kleinig et al, Z. Naturforsch 34c, 181-185 (1979); Kleinig and Schmitt, Z. Naturforsch 37c, 758-760 (1982)). A saponified extract of Methylobacterium rhodinum and of Methylomonas 16a were compared by HPLC analysis under the same conditions as mentioned above. The results are shown as follows:

Saponified M. rhodinum: inflexion at 460 nm, maxima at 487 nm, 517 nm.

Net retention volume=1.9 mL.

Saponified Methylomonas 16a: inflexion at 460 nm, maxima at 488 nm, 518 nm.

Net retention volume=2.0 mL.

### Example 14

### Enhanced Synthesis of Genetically Engineered Carotenoids in Methylomonas 16A by Amplification of Upper Isoprenoid Pathway Genes

The previous example (Example 13) demonstrated that amplification of the dxs and dxr genes in Methylomonas 16a increased the endogenous 30-carbon carotenoid content by about 30%. Amplification of dxs, dxr and other isoprenoid pathway genes, such as lytB, may be used to increase the metabolic flux into an engineered carotenoid pathway and thereby enhance production of 40-carbon carotenoids, such as β-carotene, zeaxanthin, canthaxanthin and astaxanthin. The lytB gene was amplified by PCR from Methylomonas 16a using the following primers that also introduced convenient XhoI restriction sites for subcloning:

| | |
|---|---|
| 5′-TGGCTCGAGAGTAAAACACTCAAG-3′ | (SEQ ID NO:59) |
| 5′-TAGCTCGAGTCACGCTTGC-3′ | (SEQ ID NO:60) |

The PCR conditions were: 95° C. for 5 min, 35 cycles of 95° C. for 30 sec, 47-62° C. gradient with 0.25° C. decrease/cycle for 30 sec and 72° C. for 1 min, and a final extension at 72° C. for 7 min.

Following purification, the 993 bp PCR product was digested with XhoI and ligated to pTJS75::dxs:dxr:lacZ:Tn5Kn, previously digested with XhoI and dephosphorylated with calf intestinal alkaline phosphatase. The ligated DNA was used to transform E. coli DH10B by electroporation. Analysis of the plasmid DNA from transformants selected on LB agar containing kanamycin (50 ug/ml) identified a plasmid in which the lytB gene was subcloned between the dxs and dxr genes in an operon under the control of the native dxs promoter. This operon was excised as a 4891 bp DNA fragment following sequential digestion with HindIII and BamHI restriction endonucleases, made blunt-ended by treatment with T4 DNA polymerase and purified following gel electrophoresis in 1.0% agarose (TAE). The purified DNA fragment was ligated to crt3 (Example 10) previously linearized within the crtZ gene by digestion with BstXI, made blunt-ended by treatment with T4 DNA polymerase and dephosphorylated with calf intestinal alkaline phosphatase. The ligated DNA was used to transform E. coli DH10B by electroporation and transformants were selected on LB agar containing kanamycin (50 ug/ml). Analysis of the plasmid DNA from transformants which demonstrated more intense yellow colony color than those containing crt3 identified a plasmid, designated pcrt3.2, containing both the crtEXYIB and dxs-lytB-dxr operons (FIG. 7)

HPLC analysis of extracts from E. coli containing pcrt3.2 confirmed the synthesis of β-carotene. Transfer of this plasmid into Methylomonas 16a by tri-parental conjugal mating will enhance production of β-carotene compared to transconjugants containing pcrt3.

### Example 15

### Industrial Production of β-Carotene in Methylomonas 16a

Optical Density Measurements

Growth of the Methylomonas culture was monitored at 600 nm using a Shimadzu 160U UV/Vis dual beam, recording spectrophotometer. Water was used as the blank in the reference cell. Culture samples were appropriately diluted with de-ionized water to maintain the absorbance values less than 1.0.

Dry Cell Weight Determination

20 mL of Methylomonas cell culture was filtered through a pre-weighed 0.2 μm filter (Type GTTP, Millipore, Bedford, Mass.) by vacuum filtration. Following filtration of biomass samples, filters were washed with 10 mL of de-ionized water and filtered under vacuum to dryness. Filters were then placed in a drying oven at 95° C. for 24 to 48 hr. After 24 hr, filters were cooled to room temperature and re-weighed. After recording the filter weight, the filters were returned to the drying oven and the process repeated at various time intervals until no further change in weight loss was recorded. Media contribution to the dry cell weight (DCW) measurement was obtained by filtering 20 mL of fermentation media prior to inoculation by the above procedure. Dry cell weight is calculated by the following formula:

$$DCW [ = ] [ g {mL}^{- 1} ] = \frac{\begin{matrix}[ ( weight of filter + cells ) - ( weight of filter ) ] - \\ [ ( weight of filter + media ) - ( weight of filter ) ]\end{matrix}}{20 mL culture volume}$$

Ammonia Concentration Determination

3 mL culture samples for ammonia analyses were taken from the fermenter and centrifuged at 10,000×g and 4° C. for 10 min. The supernatant was then filtered through a 0.2 μm syringe filter (Gelman Lab., Ann Arbor, Mich.) and placed at −20° C. until analyzed. Ammonia concentration in the fermentation broth was determined by ion chromatography using a Dionex System 500 Ion Chromatograph (Dionex, Sunnyvale, Calif.) equipped with a GP40 Gradient Pump, AS3500 Autosampler, and ED40 Electrochemical Detector operating in conductivity mode with an SRS current of 100 mA. Separation of ammonia was accomplished using a Dionex CS12A column fitted with a Dionex CG12A Guard column. The columns and the chemical detection cell were maintained at 35° C. Isocratic elution conditions were employed using 22 mM H₂SO₄ as the mobile phase at a flowrate of 1 mL min⁻¹. The presence of ammonia in the fermentation broth was verified by retention time comparison with an NH₄Cl standard. The concentration of ammonia in the fermentation broth was determined by comparison of area counts with a previously determined NH₄Cl standard calibration curve. When necessary, samples were diluted with de-ionized water so as to be within the bounds of the calibration curve.

Carbon Dioxide Evolution Rate (CER) Determination

The carbon dioxide concentration in the exit gas stream from the fermenter was determined by gas chromatography (GC) using a Hewlett Packard 5890 Gas Chromatograph (Hewlett Packard, Avondale, Pa.) equipped with a TCD detector and HP19091P-Q04, 32 m×32 μm×20 μm divinylbenzene/styrene porous polymer capillary column. Gas samples were withdrawn from the outlet gas stream through a sample port consisting of a polypropylene “T” to which the side arm was covered with a butyl rubber stopper. 200 μL samples were collected by piercing the rubber stopper with a Hamilton (Reno, Nev.) gas-tight GC syringe. Samples were collected after purging the barrel of the syringe a minimum of 4 times with the outlet gas. Immediately following sample collection, the volume in the syringe was adjusted to 100 μL and injected through a splitless injection port onto the column. Chromatographic conditions used for CO₂ determination were as follows: Injector Temperature (100 C); Oven Temperature (35 C); Detector Temperature (140 C); Carrier Gas (Helium); Elution Profile (Isothermal); Column Head Pressure (15 psig). The presence of CO₂ in the exit gas stream was verified by retention time comparison with a pure component CO₂ standard. The concentration of CO₂ in the exit gas stream was determined by comparison of area counts with a previously determined CO₂ standard calibration curve. Standard gas cylinders (Robert's Oxygen, Kennett Square, Pa.) containing CO₂ in the concentration range of 0.1% (v/v) to 10% (v/v) were used to generate the calibration curve.

The carbon dioxide evolution rate was calculated from the following formula:

$$CER [ = ] mmol {hr}^{- 1} = \frac{\begin{matrix}Exit Pressure * {CO}_{2} concentration * \\ inlet gas flowrate\end{matrix}}{\begin{matrix}R * Absolute temperature of \\ the exit gas stream\end{matrix}}$$

In the above equation the exit pressure from the fermenter was assumed to be equal to the atmospheric pressure. The inlet gas flowrate was calculated from the sum of the individual methane and air flowrates. R is the ideal gas constant=82.06 cm³ atm mol⁻¹ K⁻¹. The absolute temperature of the exit gas stream was calculated by the following formula: T(K)=t(° C.)+273.15, where T is the absolute temperature in K, and t is the exit gas temperature in ° C. and was assumed to be equal to the ambient temperature.

β-Carotene Extraction and Determination by High Performance Liquid Chromatography (HPLC)

15-30 mL of the Methylomonas culture was centrifuged at 10,000×g and 4° C. for 10 min. The supernatant was decanted and the cell pellet frozen at −20° C. The frozen cell pellet was thawed at room temperature to which 2.5 mL of acetone was added. The sample was vortexed for 1 min and allowed to stand at room temperature for an additional 30 min before being centrifuged at 10,000×g and 4° C. for 10 min. The acetone layer was decanted and saved. The pellet was then re-extracted with an additional 2.5 mL of acetone, centrifuged, and the two acetone pools combined. Visual observation of the cell pellet revealed that all the β-carotene had been removed from the cells following the second extraction. The acetone pool was then concentrated to 1 mL under a stream of N₂, filtered through a 0.45 μm filter, and analyzed by HPLC.

Acetone samples containing β-carotene were analyzed using a Beckman System Gold HPLC (Beckman Coulter, Fullerton, Calif.) equipped with a model 125 ternary pump system, model 168 diode array detector, and model 508 autosampler. 100 μL of concentrated acetone extracts were injected onto a HP LichroCART 125-4, C₈ reversed phase HPLC column (Hewlett Packard, Avondale, Pa.). Peaks were integrated using Beckman Gold software. Retention time and spectral comparison confirmed peak identity with β-carotene pure component standards in the wavelength range from 220 to 600 nm. The retention time and spectral profiles of the β-carotene in the acetone extracts were an exact match to those obtained from the pure component β-carotene standards. The β-carotene concentrations in the acetone extracts were quantified by comparison of area counts with a previously determined calibration curve as described below. A wavelength of 450 nm, corresponding to the maximum absorbance wavelength of β-carotene in acetone, was used for quantitation.

A mobile consisting of methanol and water was used for reversed phase separation of β-carotene. The separation of β-carotene was accomplished using a linear gradient of 60% methanol and 40% water changing linearly over 11.5 minutes to 100% methanol. Under the chromatographic conditions employed, resolution of α-carotene from β-carotene could not be attained.

β-carotene calibration curves were prepared from stock solutions by dissolving 25 mg of β-carotene (96% purity, Spectrum Chemical Inc., New Brunswick, N.J.) in 100 mL of acetone. Appropriate dilutions of this stock solution were made to span the β-carotene concentrations encountered in the acetone extracts. Calibration curves constructed in this manner were linear over the concentration range examined.

Fermentation of Methylomonas 16a

Fermentation was performed as a fed-batch fermentation under nitrogen limitation using a 3 liter, vertical, stirred tank fermenter (B. Braun Biotech Inc., Allentown, Pa.) with a working volume of 2 liters. The fermenter was equipped with 2 six-bladed Rushton turbines and stainless steel headplate with fittings for pH, temperature, and dissolved oxygen probes, inlets for pH regulating agents, sampling tube for withdrawing liquid samples, and condenser. The exit gas line from the fermenter contained a separate port for sampling the exit gas stream for GC analysis of methane, O₂, and CO₂ concentrations. The fermenter was jacketed for temperature control with the temperature maintained constant at 30° C. through the use of an external heat exchanger. Agitation was maintained in the range of 870-885 rpm. The pH of the fermentation was maintained constant at 6.95 through the use of 2.5 M NaOH and 2 M H₂SO₄.

Methane was used as the sole carbon and energy source during the fermentation. The flow of methane to the fermenter was metered using a Brooks (Brooks Instrument, Hatfield, Pa.) mass flow controller. A separate mass flow controller was used to regulate the flow of air. Prior to entering the fermenter, the individual methane and air flows were mixed and filtered through a 0.2 μm in-line filter (Millipore, Bedford, Mass.) giving a total gas flowrate of 260 mL min⁻¹ (0.13 v/v/min) and methane concentration of 23% (v/v) in the inlet gas stream. The gas was delivered to the medium 3 cm below the lower Rushton turbine through a perforated pipe. 2 liters of a minimal salts medium of the composition given in Table 14 was used for the fermentation. Silicone antifoam (Sigma Chemical Co., St. Louis, Mo.) was added to a final concentration of 800 ppm prior to sterilization to suppress foaming. Before inoculating, the fermenter and it contents were sterilized by autoclaving for 1 hr at 121° C. and 15 psia. Once the medium had cooled, 4 mL of a 25 mg mL⁻¹ kanamycin stock solution was added to the fermentation medium to maintain plasmid selection pressure during the fermentation.

TABLE 14
Fermentation Media Composition

| | Amount |
|---|---|
| Component | (g L⁻¹) |
| NH₄Cl | 1.07 |
| KH₂PO₄ | 1 |
| MgCl₂*6H₂O | 0.4 |
| CaCl₂*2H₂O | 0.2 |
| 1 M HEPES Solution | 50 mL L⁻¹ |
| (pH 7) | |
| Solution 1* | 30 mL L⁻¹ |
| Na₂SO₄ | 1 |

*Note: The compositon of Solution 1 is provided in the General Methods.

1 ml of frozen Methylomonas 16a containing plasmid pCRT1 was used to inoculate a 100 mL culture of sterile 0.5× minimal salts media containing 50 μg mL⁻¹ of kanamycin in a 500 mL Wheaton bottle sealed with a butyl rubber stopper and aluminum crimp cap. Methane was added to the culture by piercing the rubber stopper with a 60 mL syringe fitted with a 21 gauge needle to give a final methane concentration in the headspace of 25% (v/v). The inoculated medium was shaken for approximately 48 hr at 30° C. in a controlled environmental rotary shaker. When cell growth reached saturation, 5 mL of this culture was used to inoculate 2 100-mL cultures as described above. When the optical density of the cultures reached 0.8, 60 mL of each culture was used to inoculate the fermenter.

Samples were taken at 4-5 hr intervals during the course of the fermentation to monitor carotenoid production as a function of the growth phase of the organism. The specific growth rate of the culture was 0.13 hr⁻¹. No adjustment of air or methane flows was employed to prevent the culture from becoming oxygen limited during the course of the fermentation. Furthermore, the aeration and methane addition continued once the culture had stopped growing to explore β-carotene production in the absence of cell growth. Cessation of growth was indicated when no changes in optical density were observed, by the disappearance of ammonia from the fermentation media, and by an observed decrease in the CER. The β-carotene content of the cells, dry cell weight, ammonia levels, and carbon dioxide evolution rate were determined as described supra. The results are stated in Table 15 below.

TABLE 15
Fed-Batch Fermentation Results of Methylomonas sp. 16a/pCRT1

| | | | | β- | | | |
|---|---|---|---|---|---|---|---|
| | | | β- | Caro- | | | |
| | | | carotene | tene | Ammo- | | |
| | | | Titer | Titer | nia | CER^{b} | pO₂^{c} |
| Time | OD | DCW^{a} | (μg | (mg | Conc. | (mmol | (% |
| (hr) | 600 | (g L⁻¹) | gDCW⁻¹) | L⁻¹) | (mM) | hr⁻¹) | Sat'n) |
| 0.0 | 0.351 | ND^{d} | ND^{d} | ND^{d} | 23.7 | ND^{d} | ND^{d} |
| 37.7 | 1.59 | 0.54 | 2640 | 1.42 | 17.8 | 8.1 | 53.65 |
| 41.6 | 2.50 | 0.87 | 6300 | 5.51 | 13.9 | 13.2 | 33.50 |
| 45.9 | 4.27 | 1.55 | 7710 | 11.94 | 8.7 | 22.1 | 1.00 |
| 49.3 | 7.99 | 2.36 | 5050 | 12.07 | 0.12 | 19.4 | 0.0 |
| 53.5 | 11.68 | 3.44 | 4510 | 15.51 | 0 | 10.4 | 45.50 |
| 58.9 | 13.63 | 4.07 | 3960 | 15.85 | 0 | 4.2 | 65.85 |
| 63.8 | 13.80 | 3.87 | 4150 | 15.96 | 0 | 4.2 | 72.70 |
| 69.6 | 13.45 | 3.93 | 4890 | 19.01 | 0 | 2.0 | 75.30 |

^{a}DCW = [Dry Cell Weight]
^{b}CER = [Carbon Dioxide Evolution Rate]
^{c}pO2 = [Dissolved Oxygen Concentration in Fermenter]
^{d}ND = [Not Determined]

At 46 hr into the fermentation β-carotene titers reached a maximum titer of 7,710 ppm on a dry weight basis. Shortly after this time the β-carotene titer dropped substantially as the fermenter became oxygen limited as noted by the dissolved oxygen concentration. Thus, it is apparent that maintenance of high β-carotene titers is dependent on high oxygen tensions present in the fermentation media. Presumably higher β-carotene titers could be reached than reported here through better control of the dissolved oxygen concentration during the course of the fermentation. Maximum β-carotene productivities were calculated as 620 μg gDCW⁻¹ hr⁻¹ and 886 μL⁻¹ hr⁻¹. In addition, β-carotene concentrations were found to stabilize at roughly 4,400 ppm as the cells transitioned into stationary phase. It is apparent that β-carotene titers are growth associated as well as dependent on oxygen tension.

What is claimed is:

1. A method for the production of a carotenoid compound comprising:
(a) providing a transformed C1 metabolizing host cell comprising:
(i) suitable levels of isopentenyl pyrophosphate; and
(ii) at least one isolated nucleic acid molecule encoding an enzyme in the carotenoid biosynthetic pathway under the control of suitable regulatory sequences;
(b) contacting the host cell of step (a) under suitable growth conditions with an effective amount of a C1 carbon substrate whereby an carotenoid compound is produced.

2. A method according to claim 1 wherein the C1 carbon substrate is selected from the group consisting of methane, methanol, formaldehyde, formic acid, methylated amines, methylated thiols, and carbon dioxide.

3. A method according to claim 1 wherein the C1 metabolizing host cell is a methylotroph selected from the group consisting of Methylomonas, Methylobacter, Mehtylococcus, Methylosinus, Methylocyctis, Methylomicrobium, Methanomonas, Methylophilus, Methylobacillus, Methylobacterium, Hyphomicrobium, Xanthobacter, Bacillus, Paracoccus, Nocardia, Arthrobacter, Rhodopseudomonas, Pseudomonas, Candida, Hansenula, Pichia, Torulopsis, and Rhodotorula

4. A method according to claim 3 wherein C1 metabolizing host is a methanotroph.

5. A method according to claim 4 wherein the methanotroph is a methanotroph selected from the group consisting of Methylomonas, Methylobacter, Mehtylococcus, Methylosinus, Methylocyctis, Methylomicrobium, and Methanomonas.

6. A method according to claim 2 wherein the C1 carbon substrate is selected from the group consisting of methane and methanol and the C1 metabolizing host cell is a methanotroph selected from the group consisting of Methylomonas, Methylobacter, Mehtylococcus, Methylosinus, Methylocyctis, Methylomicrobium, and Methanomonas.

7. A method according to claim 6 wherein the methanotroph is a high growth methanotrophic strain which comprises a functional Embden-Meyerhof carbon pathway, said pathway comprising a gene encoding a pyrophosphate dependent phosphofructokinase enzyme.

8. A method according to claim 7 wherein the gene encoding a pyrophosphate dependent phosphofructokinase enzyme is selected from the group consisting of:
(a) an isolated nucleic acid molecule encoding the amino acid sequence as set forth in SEQ ID NO:2;
(b) an isolated nucleic acid molecule that hybridizes with (a) under the following hybridization conditions: 0.1×SSC, 0.1% SDS, 65° C. and washed with 2×SSC, 0.1% SDS followed by 0.1×SSC, 0.1% SDS;
(c) an isolated nucleic acid molecule comprising a first nucleotide sequence encoding a polypeptide of at least 437 amino acids that has at least 63% identity based on the Smith-Waterman method of alignment when compared to a polypeptide having the sequence as set forth in SEQ ID NO:2; and
(d) an isolated nucleic acid molecule that is complementary to (a), (b) or (c).

9. A method according to claim 7 wherein the high growth methanotrophic bacterial strain optionally contains at least one gene encoding a fructose bisphosphate aldolase enzyme.

10. A method according to claim 7 wherein the high growth methanotrophic bacterial strain optionally contains a functional Entner-Douderoff carbon pathway.

11. A method according to claim 8 wherein the high growth methanotrophic bacterial strain optionally contains at least one gene encoding a keto-deoxy phosphogluconate aldolase.

12. A method according to claim 9 wherein the high growth methanotrophic bacterial strain is methylomonas 16a having the ATCC designation ATCC PTA 2402.

13. A method according to claim 1 wherein the isolated nucleic acid molecule encodes a carotenoid biosynthetic enzyme selected from the group consisting of geranylgeranyl pyrophosphate (GGPP) synthase, phytoene synthase, phytoene desaturase, lycopene cyclase, β-carotene hydroxylase, zeaxanthin glucosyl transferase, β-carotene ketolase, β-carotene C-4 oxygenase, β-carotene desaturase, spheroidene monooxygenase, carotene hydratase, carotenoid 3,4-desaturase, 1-OH-carotenoid methylase, farnesyl diphosphate synthetase, and diapophytoene dehydrogenase.

14. A method according to claim 13 wherein the isolated nucleic acid molecule encoding a carotenoid biosynthetic enzyme encodes a geranylgeranyl pyrophosphate (GGPP) synthase selected from the group consisting of Genbank Acc #. AB000835, AB016043 AB019036, AB027705, AB027706, AB016044, AB034249, AB034250, AF020041, AF049658, AF049659, AF139916, AF279807, AF279808, AJ010302, AJ133724, AJ276129, D85029, L25813, L37405, U15778, U44876, X92893, X95596, X98795, and Y15112

15. A method according to claim 13 wherein the geranylgeranyl pyrophosphate (GGPP) synthase as the amino acid sequence as set forth in SEQ ID NO: 26.

16. A method according to claim 13 wherein the isolated nucleic acid molecule encoding a carotenoid biosynthetic enzyme encodes a phytoene synthase selected from the group consisting of Genbank Acc # AB001284, AB032797, AB034704, AB037975, AF009954, AF139916, AF152892, AF218415, AF220218, AJ010302, AJ133724, AJ278287, AJ304825, AJ308385, D58420, L23424, L25812, L37405, M38424, M87280, S71770, U32636, U62808, U87626, U91900, X52291, X60441, X63873, X68017, X69172, and X78814.

17. A method according to claim 13 wherein the phytoene synthase as the amino acid sequence as set forth in SEQ ID NO:34.

18. A method according to claim 13 wherein the isolated nucleic acid molecule encoding a carotenoid biosynthetic enzyme encodes a phytoene desaturase selected from the group consisting of Genbank Acc #AB046992, AF039585, AF049356, AF139916, AF218415AF251014, AF364515, D58420, D83514, L16237, L37405, M64704, M88683, S71770, U37285, U46919, U62808, X55289, X59948, X62574, X68058, X71023, X78271, X78434, X78815, X86783, Y14807, Y15007, Y15112, Y15114, and Z11165

19. A method according to claim 13 wherein the phytoene desaturase as the amino acid sequence as set forth in SEQ ID NO:32

20. A method according to claim 13 wherein the isolated nucleic acid molecule encoding a carotenoid biosynthetic enzyme encodes a lycopene cyclase selected from the group consisting of Genbank Acc # AF139916, AF152246, AF218415, AF272737, AJ133724, AJ250827, AJ276965, D58420, D83513, L40176, M87280, U50738, U50739 U62808, X74599, X81787, X86221, X86452, X95596, and X98796.

21. A method according to claim 13 wherein the lycopene cyclase as the amino acid sequence as set forth in SEQ ID NO:30

22. A method according to claim 13 wherein the isolated nucleic acid molecule encoding a carotenoid biosynthetic enzyme encodes a β-carotene hydroxylase selected from the group consisting of Genbank Acc # D58420, D58422, D90087, M87280, U62808, Y15112,

23. A method according to claim 13 wherein β-carotene hydroxylase as the amino acid sequence as set forth in SEQ ID NO:36

24. A method according to claim 13 wherein the isolated nucleic acid molecule encoding a carotenoid biosynthetic enzyme encodes zeaxanthin glucosyl transferase selected from the group consisting of Genbank Acc #. D90087, M87280, and M90698.

25. A method according to claim 13 wherein zeaxanthin glucosyl transferase as the amino acid sequence as set forth in SEQ ID NO:28

26. A method according to claim 13 wherein the isolated nucleic acid molecule encoding a carotenoid biosynthetic enzyme encodes a β-carotene ketolase selected from the group consisting of Genbank Acc #. AF218415, D45881, D58420, D58422, X86782, and Y15112.

27. A method according to claim 13 wherein the isolated nucleic acid molecule encoding a carotenoid biosynthetic enzyme encodes a β-carotene ketolase having the amino acid sequence as set for the in SEQ ID NO:38.

28. A method according to claim 13 wherein the isolated nucleic acid molecule encoding a carotenoid biosynthetic enzyme encodes a β-carotene C-4 oxygenase selected from the group consisting of Genbank Acc #. X86782, and Y15112.

29. A method according to claim 13 wherein the isolated nucleic acid molecule encoding a carotenoid biosynthetic enzyme encodes a β-carotene desaturase selected from the group consisting of Genbank Acc #. AF047490, AF121947, AF139916, AF195507, AF272737, IFO13350, AF372617, AJ133724, AJ224683, D26095 U38550, X89897, Y15115, and PCC7210,

30. A method according to claim 13 wherein the isolated nucleic acid molecule encoding a carotenoid biosynthetic enzyme encodes a spheroidene monooxygenase selected from the group consisting of Genbank Acc #. AJ010302, Z11165, and X52291.

31. A method according to claim 13 wherein the isolated nucleic acid molecule encoding a carotenoid biosynthetic enzyme encodes a carotene hydratase selected from the group consisting of Genbank Acc #. AB034704, AF195122, AJ010302, AF287480, U73944, X52291, Z11165, and Z21955.

32. A method according to claim 13 wherein the isolated nucleic acid molecule encoding a carotenoid biosynthetic enzyme encodes a carotenoid 3,4-desaturase selected from the group consisting of Genbank Acc#. AJ010302, X63204 U73944, X52291, and Z11165,

33. A method according to claim 13 wherein the isolated nucleic acid molecule encoding a carotenoid biosynthetic enzyme encodes a 10H-carotenoid methylase selected from the group consisting of Genbank Acc #. AB034704, AF288602, AJ010302, X52291 and Z11165.

34. A method according to claim 13 wherein the isolated nucleic acid molecule encoding a carotenoid biosynthetic enzyme encodes a farnesyl diphosphate synthetase selected from the group consisting of Genbank Acc #. AB003187, AB016094, AB021747, AB028044, AB028046, AB028047, AF112881, AF136602, AF384040, D00694, D13293, D85317, X75789, Y12072, Z49786, U80605, X76026, X82542×82543, AF234168, L46349, L46350, L46367, M89945, NM_{—}002004, U36376, XM_{—}034497, XM_{—}034498, XM_{—}034499, and XM_{—}034500.

35. A method according to claim 13 wherein the isolated nucleic acid molecule encoding a carotenoid biosynthetic enzyme encodes a farnesyl diphosphate synthetase having the amino acid sequence as set forth in SEQ ID NO:20.

36. A method according to claim 13 wherein the isolated nucleic acid molecule encoding a carotenoid biosynthetic enzyme encodes a diapophytoene dehydrogenase enzyme as described by Genbank Acc #. X73889.

37. A method according to claim 13 wherein the isolated nucleic acid molecule encoding a carotenoid biosynthetic enzyme encodes a diapophytoene dehydrogenase enzyme having the amino acid sequence selected from the group consisting of SEQ ID NO:22 and SEQ ID NO:24.

38. A method according to claim 1 wherein said methanotrophic bacteria is methylomonas 16a ATCC PTA 2402.

39. A method according to claim 1 wherein the suitable levels of isopentenyl pyrophosphate are provided by the expression heterologus upper pathway isoprenoid pathway genes.

40. A method according to claim 39 wherein said upper pathway isoprenoid genes are selected from the group consisting of D-1-deoxyxylulose-5-phosphate synthase (Dxs), D-1-deoxyxylulose-5-phosphate reductoisomerase (Dxr), 2C-methyl-d-erythritol cytidylyltransferase (IspD), 4-diphosphocytidyl-2-C-methylerythritol kinase (IspE), 2C-methyl-d-erythritol 2,4-cyclodiphosphate synthase (IspF), CTP synthase (PyrG), lytB, and GcpE.

41. A method according to claim 40 wherein said gene encoding a D-1-deoxyxylulose-5-phosphate synthase (Dxs), encodes a polypeptide as set forth in SEQ ID NO:6

42. A method according to claim 40 wherein said gene encoding a D-1-deoxyxylulose-5-phosphate reductoisomerase (Dxr), encodes a polypeptide as set forth in SEQ ID NO:8.

43. A method according to claim 40 wherein said gene encoding a 2C-methyl-d-erythritol cytidylyltransferase (IspD), encodes a polypeptide as set forth in SEQ ID NO:10.

44. A method according to claim 40 wherein said gene encoding a 4-diphosphocytidyl-2-C-methylerythritol kinase (IspE), encodes a polypeptide as set forth in SEQ ID NO:12.

45. A method according to claim 40 wherein said gene encoding a 2C-methyl-d-erythritol 2,4-cyclodiphosphate synthase (IspF), encodes a polypeptide as set forth in SEQ ID NO:14.

46. A method according to claim 40 wherein said gene encoding a CTP synthase (PyrG) encodes a polypeptide as set forth in SEQ ID NO:16.

47. A method according to claim 40 wherein said gene encoding an activity for the production of dimethylallyl diphosphate (lytB) encodes a polypeptide as set forth in SEQ ID NO:18.

48. A method according to claim 1 wherein the carotenoid compound is selected form the group consisting of Antheraxanthin, adonixanthin, Astaxanthin, Canthaxanthin, capsorubrin, β-cryptoxanthin alpha-carotene, beta-carotene, epsilon-carotene, echinenone, gamma-carotene, zeta-carotene, alpha-cryptoxanthin, diatoxanthin, 7,8-didehydroastaxanthin, fucoxanthin, fucoxanthinol, isorenieratene, lactucaxanthin, lutein, lycopene, neoxanthin, neurosporene, hydroxyneurosporene, peridinin, phytoene, rhodopin, rhodopin glucoside, siphonaxanthin, spheroidene, spheroidenone, spirilloxanthin, uriolide, uriolide acetate, violaxanthin, zeaxanthin-β-diglucoside, and zeaxanthin.

49. A method for the over-production of carotenoid production in a transformed C1 metabolizing host comprising:
(a) providing a transformed C1 metabolizing host cell comprising:
(i) suitable levels of isopentenyl pyrophosphate; and
(ii) at least one isolated nucleic acid molecule encoding an enzyme in the carotenoid biosynthetic pathway under the control of suitable regulatory sequences; and
(iii) either:
1) multiple copies of at least one gene encoding an enzyme selected from the group consisting of D-1-deoxyxylulose-5-phosphate synthase (Dxs), D-1-deoxyxylulose-5-phosphate reductoisomerase (Dxr), 2C-methyl-d-erythritol cytidylyltransferase (IspD), 4-diphosphocytidyl-2-C-methylerythritol kinase (IspE), 2C-methyl-d-erythritol 2,4-cyclodiphosphate synthase (IspF), CTP synthase (PyrG) lytB and gcpE; or
2) at least one gene encoding an enzyme selected from the group consisting of D-1-deoxyxylulose-5-phosphate synthase (Dxs), D-1-deoxyxylulose-5-phosphate reductoisomerase (Dxr), 2C-methyl-d-erythritol cytidylyltransferase (IspD), 4-diphosphocytidyl-2-C-methylerythritol kinase (IspE), 2C-methyl-d-erythritol 2,4-cyclodiphosphate synthase (IspF), CTP synthase (PyrG), lytB and gcpE operably linked to a strong promoter.
(b) contacting the host cell of step (a) under suitable growth conditions with an effective amount of a C1 carbon substrate whereby a carotenoid compound is over-produced.

50. A method according to claim 49 wherein the at least one gene encoding an enzyme of either part (a)(iii)(1) or (a)(iii)(2) encodes an enzyme selected from the group consisting of SEQ ID NO:6, 8, 10, 12, 14, 16, and 18.
